(19) United States
(12) Patent Application Publication
(10) Pub. No.: US 2024/0271161 A1
(43) Pub. Date: Aug. 15, 2024

(54) NOVEL RECOMBINASES AND METHODS OF USE

(71) Applicant: Editas Medicine, Inc., Cambridge, MA (US)

(72) Inventors: Eugenio Marco Rubio, West Roxbury, MA (US); Meng Wu, Wilmington, MA (US); Joseph Connor Neumeyer, Cambridge, MA (US); Hui Li, Apex, NC (US)

(21) Appl. No.: 18/468,681

(22) Filed: Sep. 15, 2023

Related U.S. Application Data
(60) Provisional application No. 63/480,342, filed on Jan. 18, 2023.
(60) Provisional application No. 63/376,048, filed on Sep. 16, 2022.

(51) Int. Cl. C12N 15/90; C12N 9/12

(52) U.S. Cl. CPC C12N 15/90; C12N 9/1241

(57) ABSTRACT

The present disclosure provides novel large serine recombinases and their cognate attachment sites in the human genome. Methods for using these large serine recombinases and attachment sites are also provided herein.

### CROSS-REFERENCE TO RELATED APPLICATIONS

This application claims the benefit of U.S. Provisional Application No. 63/376,048, filed Sep. 16, 2022, and U.S. Provisional Application No. 63/480,342, filed Jan. 18, 2023, the contents of which are hereby incorporated herein by reference in their entirety.

### SEQUENCE LISTING

The present specification makes reference to a Sequence Listing (submitted electronically as an .xml file named “2011271-0250_SL.xml” on Sep. 15, 2023). The .xml file was generated on Dec. 21, 2022 and is 64,316,640 bytes in size. The entire contents of the Sequence Listing are herein incorporated by reference.

### Large Table

The present specification makes reference to Table 1 (submitted electronically as a .txt file named “Table_1. txt” on Sep. 15, 2023). The .txt file was generated on Sep. 14, 2023 and is 3,033,378 bytes in size. The entire contents of Table 1 are herein incorporated by reference.

### BACKGROUND

Site-specific recombination involves the specialized movement of nucleotide sequences between non-homologous sites within a genome or between genomes (e.g., between phage and bacterial genomes). Mobilization of these genetic elements can occur within a single chromosome or between two different chromosomes, giving rise to variations essential for adaptation and evolution. Site-specific recombination is guided by site-specific recombinases, which are most abundant among prokaryotes and lower eukaryotes (Alberts et al. 2002). Site-specific recombinases recognize two specific “attachment” sites present on one or both DNA molecules, catalyze the cleavage of specific phosphodiester bonds within these two attachment sites, and rejoin the broken ends to form recombinants (Olorunniji et al. 2016). This process doesn't require extensive DNA homology, as homologous recombination (HR) does, nor does it involve any DNA synthesis or degradation. As such, this form of recombination is often referred to as conservative site-specific recombination.

The vast majority of conservative site-specific recombinases fall into two families: tyrosine recombinases and serine recombinases. Each family is named according to the identity of the active nucleophilic amino acid residue responsible for attacking the DNA phosphodiester bonds to create strand breaks, and subsequent formation of a covalent linkage to conserve bond energy for recombination (Olorunniji et al. 2016). While there are a number of features shared by both families, their proteins have diverging sequences and are structurally distinct. Furthermore, both families operate on divergent recombination mechanisms.

Tyrosine recombinases have been widely identified in a number of bacteriophage, prokaryotes, fungi, and ciliates. Prominent tyrosine recombinases include Cre, Flp, XerD, HP1 integrase and A integrase (Swalla et al. 2003). Tyrosine recombinases engage in breaking, exchanging, and rejoining the DNA strands two at a time, which results in formation of a “Holliday junction” or four-way junction intermediate. Many tyrosine recombinases, including Cre and Flp, promote recombination between two identical sites, which encourages continual recombination that may result in returning the DNA back to an undesired non-recombinant form. A number of tyrosine recombinases from bacteriophage recombine at non-identical sites (e.g., 2 integrase), but unfortunately require large complex attachment sites making them less useful for clinical applications (Olorunniji et al. 2016).

Serine recombinases are found in viruses, bacteria, and archaea. Unlike tyrosine recombinases, serine recombinases do not make a Holliday junction or four-way junction intermediate during recombination. Instead, they recognize and bind at two different short attachment sites, known as attP (in a phage genome) and attB (in a bacterial genome), to form a tetrameric synaptic complex. Dual stranded breaks occur simultaneously, and recombination is brought about by a unique subunit rotation mechanism of the cut DNA ends. Recombination results in newly modified sites known as attL and attR, which cannot be excised by site-specific recombination alone and require a phage-encoded recombination directionality factor (RDF) (Van Duyne et al. 2013; Olorunniji et al. 2016). As a result, serine recombinases lead to recombination that is unidirectional and irreversible, preventing inadvertent additional recombination events.

The unidirectional and irreversible nature of the modifications that result from serine recombinases can make them suitable candidates for insertion, deletion, and reconfiguration of substantial segments of DNA. Under optimal conditions, the short, highly specific attachment sites (about 40-50 bp) are conducive to near 100% conversion of substrates to recombinant products in a matter of a few minutes both in vitro and in vivo (Olorunniji et al. 2016; Van Duyne et al. 2013). While attractive for genetic manipulation, there are still considerable challenges in clinical application of serine recombinases. The present disclosure provided herein seeks to address these challenges.

### SUMMARY OF THE INVENTION

The present disclosure provides, inter alia, newly identified large serine recombinases included in Table 1 (and Table 2 and Table 3) and identifies and characterizes their respective attachment sites (attB and attP) and exemplary predicted donor sites (attD) and attachment sites in the human genome (attH). The disclosed recombinases, attachment sites, compositions, and methods enable the targeted integration of desired DNA payloads into specific sequences within the human genome, for example, for the purposes of gene therapy.

In one aspect, the present disclosure provides methods for integrating an exogenous nucleic acid (e.g., an exogenous DNA) into a genome (e.g., a human genome), the method comprising: contacting a cell (e.g., a human cell) with an exogenous nucleic acid (e.g., an exogenous DNA) comprising a nucleic acid sequence of interest and a first attachment site and a serine recombinase or a polynucleotide encoding the serine recombinase, wherein the genome (e.g., human genome) comprises a second attachment site and recombination between the first and second attachment sites results in integration of the exogenous nucleic acid (e.g., exogenous DNA) into the genome (e.g., a human genome). In some embodiments, the cell may be a non-human cell, e.g., a bacterial cell and the targeted genome may be a non-human genome, e.g., a bacterial genome. For example, in some embodiments the methods of the present disclosure may be used to integrate an exogenous nucleic acid into the genome of a bacterial cell in the gut of a human subject.

In some embodiments, exogenous nucleic acid (e.g., exogenous DNA) is up to 5 kb, up to 25 kb, up to 50 kb, up to 75 kb, up to 100 kb, up to 150 kb, up to 200 kb, up to 250 kb, or up to 300 kb in size.

In some embodiments, a first attachment site is or comprises a donor attachment (attD) site. In some embodiments, an attD site comprises an attB sequence or an attP sequence. In some embodiments, a first attachment site comprises a nucleic acid sequence at least 50% identical to an attB or attP sequence selected from Table 1. In some embodiments, a first attachment site comprises a nucleic acid sequence at least 50% identical to an attB or attP sequence selected from Table 2. In some embodiments, a first attachment site comprises a nucleic acid sequence at least 50% identical to an attB or attP sequence selected from Table 3.

In some embodiments, a second attachment site is or comprises an acceptor attachment (attA) site. In some embodiments, an attA site comprises an attB sequence, an attP sequence, or an attH sequence. In some embodiments, a second attachment site comprises a nucleic acid sequence at least 50% identical to: an attB sequence selected from Table 1, an attP sequence selected from Table 1, or an attH sequence selected from Table 1. In some embodiments, a second attachment site comprises a nucleic acid sequence at least 50% identical to: an attB sequence selected from Table 2, an attP sequence selected from Table 2, or an attH sequence selected from Table 2. In some embodiments, a second attachment site comprises a nucleic acid sequence at least 50% identical to: an attB sequence selected from Table 3, an attP sequence selected from Table 3, or an attH sequence selected from Table 3.

In some embodiments, a serine recombinase comprises an amino acid sequence at least 80% identical to a sequence selected from Table 1. In some embodiments, a serine recombinase comprises an amino acid sequence at least 80% identical to a sequence selected from Table 2. In some embodiments, a serine recombinase comprises an amino acid sequence at least 80% identical to a sequence selected from Table 3.

The method of any one of the preceding claims, wherein the serine recombinase comprises: an amino-terminal catalytic domain, a recombinase domain, and a DNA-binding zinc ribbon domain, wherein, according to UCLUST algorithm analysis, the amino-terminal catalytic domain, the recombinase domain, and the DNA-binding zinc ribbon domain comprise amino acid sequences at least 90% identical to a sequence selected from Table 1, wherein the sequence selected from Table 1 comprises an amino-terminal catalytic domain, a recombinase domain, and a DNA-binding zinc ribbon domain. As used herein the terms “according to UCLUST algorithm analysis” mean that the reference and query sequences were analyzed using the UCLUST algorithm (see Edgar 2010 and rive5.com/usearch/manual/uclust_algo.html) with default parameters and the cluster_fast command (e.g., usearch-cluster_fast reads.fasta-centroids c.fasta-id 0.90 if seeking to identify sequences with at least 90% identity according to UCLUST algorithm analysis). See also drive5.com/usearch/manual/cmd_cluster_fast.html and drive5.com/usearch/manual/opt_id.html for further details.

The method of any one of the preceding claims, wherein the serine recombinase comprises: an amino-terminal catalytic domain, a recombinase domain, and a DNA-binding zinc ribbon domain, wherein, according to UCLUST algorithm analysis, the amino-terminal catalytic domain, the recombinase domain, and the DNA-binding zinc ribbon domain comprise amino acid sequences at least 90% identical to a sequence selected from Table 1, wherein the sequence selected from Table 2 comprises an amino-terminal catalytic domain, a recombinase domain, and a DNA-binding zinc ribbon domain.

In some embodiments, a serine recombinase is a recombinase selected from cluster 1 as identified in Table 1. In some embodiments, a serine recombinase is a recombinase selected from cluster 2 as identified in Table 1. In some embodiments, a serine recombinase is a recombinase selected from cluster 3 as identified in Table 1. In some embodiments, a serine recombinase is a recombinase selected from cluster 4 as identified in Table 1. In some embodiments, a serine recombinase is a recombinase selected from cluster 5 as identified in Table 1. In some embodiments, a serine recombinase is a recombinase selected from cluster 6 as identified in Table 1. In some embodiments, a serine recombinase is a recombinase selected from cluster 7 as identified in Table 1. In some embodiments, a serine recombinase is a recombinase selected from cluster 8 as identified in Table 1. In some embodiments, a serine recombinase is a recombinase selected from cluster 9 as identified in Table 1. In some embodiments, a serine recombinase is a recombinase selected from cluster 10 as identified in Table 1. In some embodiments, a serine recombinase is a recombinase selected from cluster 11 as identified in Table 1. In some embodiments, a serine recombinase is a recombinase selected from cluster 12 as identified in Table 1. In some embodiments, a serine recombinase is a recombinase selected from cluster 13 as identified in Table 1. In some embodiments, a serine recombinase is a recombinase selected from cluster 14 as identified in Table 1. In some embodiments, a serine recombinase is a recombinase selected from cluster 15 as identified in Table 1. In some embodiments, a serine recombinase is a recombinase selected from cluster 16 as identified in Table 1. In some embodiments, a serine recombinase is a recombinase selected from cluster 17 as identified in Table 1. In some embodiments, a serine recombinase is a recombinase selected from cluster 18 as identified in Table 1. In some embodiments, a serine recombinase is a recombinase selected from cluster 19 as identified in Table 1. In some embodiments, a serine recombinase is a recombinase selected from cluster 20 as identified in Table 1. In some embodiments, a serine recombinase is a recombinase selected from cluster 21 as identified in Table 1. In some embodiments, a serine recombinase is a recombinase selected from cluster 22 as identified in Table 1. In some embodiments, a serine recombinase is a recombinase selected from cluster 23 as identified in Table 1. In some embodiments, a serine recombinase is a recombinase selected from cluster 24 as identified in Table 1. In some embodiments, a serine recombinase is a recombinase selected from cluster 25 as identified in Table 1. In some embodiments, a serine recombinase is a recombinase selected from cluster 26 as identified in Table 1. In some embodiments, a serine recombinase is a recombinase selected from cluster 27 as identified in Table 1. In some embodiments, a serine recombinase is a recombinase selected from cluster 28 as identified in Table 1. In some embodiments, a serine recombinase is a recombinase selected from cluster 29 as identified in Table 1. In some embodiments, a serine recombinase is a recombinase selected from cluster 30 as identified in Table 1. In some embodiments, a serine recombinase is a recombinase selected from cluster 31 as identified in Table 1. In some embodiments, a serine recombinase is a recombinase selected from cluster 32 as identified in Table 1. In some embodiments, a serine recombinase is a recombinase selected from cluster 33 as identified in Table 1. In some embodiments, a serine recombinase is a recombinase selected from cluster 34 as identified in Table 1. In some embodiments, a serine recombinase is a recombinase selected from cluster 35 as identified in Table 1. In some embodiments, a serine recombinase is a recombinase selected from cluster 36 as identified in Table 1. In some embodiments, a serine recombinase is a recombinase selected from cluster 37 as identified in Table 1. In some embodiments, a serine recombinase is a recombinase selected from cluster 38 as identified in Table 1. In some embodiments, a serine recombinase is a recombinase selected from cluster 39 as identified in Table 1. In some embodiments, a serine recombinase is a recombinase selected from cluster 40 as identified in Table 1. In some embodiments, a serine recombinase is a recombinase selected from cluster 41 as identified in Table 1. In some embodiments, a serine recombinase is a recombinase selected from cluster 42 as identified in Table 1. In some embodiments, a serine recombinase is a recombinase selected from cluster 43 as identified in Table 1. In some embodiments, a serine recombinase is a recombinase selected from cluster 44 as identified in Table 1. In some embodiments, a serine recombinase is a recombinase selected from cluster 45 as identified in Table 1. In some embodiments, a serine recombinase is a recombinase selected from cluster 46 as identified in Table 1. In some embodiments, a serine recombinase is a recombinase selected from cluster 47 as identified in Table 1. In some embodiments, a serine recombinase is a recombinase selected from cluster 48 as identified in Table 1. In some embodiments, a serine recombinase is a recombinase selected from cluster 49 as identified in Table 1. In some embodiments, a serine recombinase is a recombinase selected from cluster 50 as identified in Table 1. In some embodiments, a serine recombinase is a recombinase selected from cluster 51 as identified in Table 1. In some embodiments, a serine recombinase is a recombinase selected from cluster 52 as identified in Table 1. In some embodiments, a serine recombinase is a recombinase selected from cluster 53 as identified in Table 1. In some embodiments, a serine recombinase is a recombinase selected from cluster 54 as identified in Table 1. In some embodiments, a serine recombinase is a recombinase selected from cluster 55 as identified in Table 1. In some embodiments, a serine recombinase is a recombinase selected from cluster 56 as identified in Table 1. In some embodiments, a serine recombinase is a recombinase selected from cluster 57 as identified in Table 1. In some embodiments, a serine recombinase is a recombinase selected from cluster 58 as identified in Table 1. In some embodiments, a serine recombinase is a recombinase selected from cluster 59 as identified in Table 1. In some embodiments, a serine recombinase is a recombinase selected from cluster 60 as identified in Table 1. In some embodiments, a serine recombinase is a recombinase selected from cluster 61 as identified in Table 1. In some embodiments, a serine recombinase is a recombinase selected from cluster 62 as identified in Table 1. In some embodiments, a serine recombinase is a recombinase selected from cluster 63 as identified in Table 1. In some embodiments, a serine recombinase is a recombinase selected from cluster 64 as identified in Table 1. In some embodiments, a serine recombinase is a recombinase selected from cluster 65 as identified in Table 1. In some embodiments, a serine recombinase is a recombinase selected from cluster 66 as identified in Table 1. In some embodiments, a serine recombinase is a recombinase selected from cluster 67 as identified in Table 1. In some embodiments, a serine recombinase is a recombinase selected from cluster 68 as identified in Table 1. In some embodiments, a serine recombinase is a recombinase selected from cluster 69 as identified in Table 1. In some embodiments, a serine recombinase is a recombinase selected from cluster 70 as identified in Table 1. In some embodiments, a serine recombinase is a recombinase selected from cluster 71 as identified in Table 1. In some embodiments, a serine recombinase is a recombinase selected from cluster 72 as identified in Table 1. In some embodiments, a serine recombinase is a recombinase selected from cluster 73 as identified in Table 1. In some embodiments, a serine recombinase is a recombinase selected from cluster 74 as identified in Table 1. In some embodiments, a serine recombinase is a recombinase selected from cluster 75 as identified in Table 1. In some embodiments, a serine recombinase is a recombinase selected from cluster 76 as identified in Table 1. In some embodiments, a serine recombinase is a recombinase selected from cluster 77 as identified in Table 1. In some embodiments, a serine recombinase is a recombinase selected from cluster 78 as identified in Table 1. In some embodiments, a serine recombinase is a recombinase selected from cluster 79 as identified in Table 1. In some embodiments, a serine recombinase is a recombinase selected from cluster 80 as identified in Table 1. In some embodiments, a serine recombinase is a recombinase selected from cluster 81 as identified in Table 1. In some embodiments, a serine recombinase is a recombinase selected from cluster 82 as identified in Table 1. In some embodiments, a serine recombinase is a recombinase selected from cluster 83 as identified in Table 1. In some embodiments, a serine recombinase is a recombinase selected from cluster 84 as identified in Table 1. In some embodiments, a serine recombinase is a recombinase selected from cluster 85 as identified in Table 1. In some embodiments, a serine recombinase is a recombinase selected from cluster 86 as identified in Table 1. In some embodiments, a serine recombinase is a recombinase selected from cluster 87 as identified in Table 1. In some embodiments, a serine recombinase is a recombinase selected from cluster 88 as identified in Table 1. In some embodiments, a serine recombinase is a recombinase selected from cluster 89 as identified in Table 1. In some embodiments, a serine recombinase is a recombinase selected from cluster 90 as identified in Table 1. In some embodiments, a serine recombinase is a recombinase selected from cluster 91 as identified in Table 1. In some embodiments, a serine recombinase is a recombinase selected from cluster 92 as identified in Table 1. In some embodiments, a serine recombinase is a recombinase selected from cluster 93 as identified in Table 1. In some embodiments, a serine recombinase is a recombinase selected from cluster 94 as identified in Table 1. In some embodiments, a serine recombinase is a recombinase selected from cluster 95 as identified in Table 1. In some embodiments, a serine recombinase is a recombinase selected from cluster 96 as identified in Table 1. In some embodiments, a serine recombinase is a recombinase selected from cluster 97 as identified in Table 1. In some embodiments, a serine recombinase is a recombinase selected from cluster 98 as identified in Table 1. In some embodiments, a serine recombinase is a recombinase selected from cluster 99 as identified in Table 1. In some embodiments, a serine recombinase is a recombinase selected from cluster 100 as identified in Table 1. In some embodiments, a serine recombinase is a recombinase selected from cluster 101 as identified in Table 1. In some embodiments, a serine recombinase is a recombinase selected from cluster 102 as identified in Table 1. In some embodiments, a serine recombinase is a recombinase selected from cluster 103 as identified in Table 1. In some embodiments, a serine recombinase is a recombinase selected from cluster 104 as identified in Table 1. In some embodiments, a serine recombinase is a recombinase selected from cluster 105 as identified in Table 1. In some embodiments, a serine recombinase is a recombinase selected from cluster 106 as identified in Table 1. In some embodiments, a serine recombinase is a recombinase selected from cluster 107 as identified in Table 1. In some embodiments, a serine recombinase is a recombinase selected from cluster 108 as identified in Table 1. In some embodiments, a serine recombinase is a recombinase selected from cluster 109 as identified in Table 1. In some embodiments, a serine recombinase is a recombinase selected from cluster 110 as identified in Table 1. In some embodiments, a serine recombinase is a recombinase selected from cluster 111 as identified in Table 1. In some embodiments, a serine recombinase is a recombinase selected from cluster 112 as identified in Table 1. In some embodiments, a serine recombinase is a recombinase selected from cluster 113 as identified in Table 1. In some embodiments, a serine recombinase is a recombinase selected from cluster 114 as identified in Table 1. In some embodiments, a serine recombinase is a recombinase selected from cluster 115 as identified in Table 1. In some embodiments, a serine recombinase is a recombinase selected from cluster 116 as identified in Table 1. In some embodiments, a serine recombinase is a recombinase selected from cluster 117 as identified in Table 1. In some embodiments, a serine recombinase is a recombinase selected from cluster 118 as identified in Table 1. In some embodiments, a serine recombinase is a recombinase selected from cluster 119 as identified in Table 1. In some embodiments, a serine recombinase is a recombinase selected from cluster 120 as identified in Table 1. In some embodiments, a serine recombinase is a recombinase selected from cluster 121 as identified in Table 1. In some embodiments, a serine recombinase is a recombinase selected from cluster 122 as identified in Table 1. In some embodiments, a serine recombinase is a recombinase selected from cluster 123 as identified in Table 1. In some embodiments, a serine recombinase is a recombinase selected from cluster 124 as identified in Table 1. In some embodiments, a serine recombinase is a recombinase selected from cluster 125 as identified in Table 1. In some embodiments, a serine recombinase is a recombinase selected from cluster 126 as identified in Table 1. In some embodiments, a serine recombinase is a recombinase selected from cluster 127 as identified in Table 1. In some embodiments, a serine recombinase is a recombinase selected from cluster 128 as identified in Table 1. In some embodiments, a serine recombinase is a recombinase selected from cluster 129 as identified in Table 1. In some embodiments, a serine recombinase is a recombinase selected from cluster 130 as identified in Table 1. In some embodiments, a serine recombinase is a recombinase selected from cluster 131 as identified in Table 1. In some embodiments, a serine recombinase is a recombinase selected from cluster 132 as identified in Table 1. In some embodiments, a serine recombinase is a recombinase selected from cluster 133 as identified in Table 1. In some embodiments, a serine recombinase is a recombinase selected from cluster 134 as identified in Table 1. In some embodiments, a serine recombinase is a recombinase selected from cluster 135 as identified in Table 1. In some embodiments, a serine recombinase is a recombinase selected from cluster 136 as identified in Table 1. In some embodiments, a serine recombinase is a recombinase selected from cluster 137 as identified in Table 1. In some embodiments, a serine recombinase is a recombinase selected from cluster 138 as identified in Table 1. In some embodiments, a serine recombinase is a recombinase selected from cluster 139 as identified in Table 1. In some embodiments, a serine recombinase is a recombinase selected from cluster 140 as identified in Table 1. In some embodiments, a serine recombinase is a recombinase selected from cluster 141 as identified in Table 1. In some embodiments, a serine recombinase is a recombinase selected from cluster 142 as identified in Table 1. In some embodiments, a serine recombinase is a recombinase selected from cluster 143 as identified in Table 1. In some embodiments, a serine recombinase is a recombinase selected from cluster 144 as identified in Table 1. In some embodiments, a serine recombinase is a recombinase selected from cluster 145 as identified in Table 1. In some embodiments, a serine recombinase is a recombinase selected from cluster 146 as identified in Table 1. In some embodiments, a serine recombinase is a recombinase selected from cluster 147 as identified in Table 1. In some embodiments, a serine recombinase is a recombinase selected from cluster 148 as identified in Table 1. In some embodiments, a serine recombinase is a recombinase selected from cluster 149 as identified in Table 1. In some embodiments, a serine recombinase is a recombinase selected from cluster 150 as identified in Table 1. In some embodiments, a serine recombinase is a recombinase selected from cluster 151 as identified in Table 1. In some embodiments, a serine recombinase is a recombinase selected from cluster 152 as identified in Table 1. In some embodiments, a serine recombinase is a recombinase selected from cluster 153 as identified in Table 1. In some embodiments, a serine recombinase is a recombinase selected from cluster 154 as identified in Table 1. In some embodiments, a serine recombinase is a recombinase selected from cluster 155 as identified in Table 1. In some embodiments, a serine recombinase is a recombinase selected from cluster 156 as identified in Table 1. In some embodiments, a serine recombinase is a recombinase selected from cluster 157 as identified in Table 1. In some embodiments, a serine recombinase is a recombinase selected from cluster 158 as identified in Table 1. In some embodiments, a serine recombinase is a recombinase selected from cluster 159 as identified in Table 1.

In some embodiments, a serine recombinase comprises an amino acid sequence at least 80% identical to a sequence selected from SEQ ID NO: 58926, SEQ ID NO: 10611, SEQ ID NO: 33021, SEQ ID NO: 40191, SEQ ID NO: 5681, SEQ ID NO: 36231, SEQ ID NO: 34841, SEQ ID NO: 9906, SEQ ID NO: 21701, SEQ ID NO: 7466, SEQ ID NO: 57456, SEQ ID NO: 41066, SEQ ID NO: 41186, SEQ ID NO: 21126, SEQ ID NO: 1191, SEQ ID NO: 35081, SEQ ID NO: 18926, SEQ ID NO: 51806, SEQ ID NO: 58376, SEQ ID NO: 29771, SEQ ID NO: 21276, or SEQ ID NO: 36986.

In some embodiments, a serine recombinase, a first attachment site, and a second attachment site comprise sequences at least 80% identical to sequences that have the same system ID in Table 1. In some embodiments, a serine recombinase, a first attachment site, and a second attachment site comprise sequences at least 80% identical to sequences that have the same system ID in Table 2. In some embodiments, a serine recombinase, a first attachment site, and a second attachment site comprise sequences at least 80% identical to sequences that have the same system ID in Table 3.

In some embodiments, a polynucleotide encoding a serine recombinase is or comprises mRNA. In some embodiments, a polynucleotide encoding a serine recombinase is or comprises DNA.

In some embodiments, a polynucleotide encoding a serine recombinase is operably linked to a promoter that is active in a human cell.

In some embodiments, an exogenous nucleic acid (e.g., exogenous DNA) is or comprises a plasmid, a nanoplasmid, a mini-circle, or doggybone DNA (dbDNA).

In some embodiments, an exogenous nucleic acid (e.g., exogenous DNA) is delivered to a human cell in a lipid nanoparticle (LNP), an adeno-associated virus (AAV), a lentivirus, a virus-like particle (VLP), an exosome, a cationic nanoparticle, or a dendrimer. In some embodiments, an exogenous DNA and a polynucleotide encoding a serine recombinase are delivered to a human cell in an LNP, and wherein the polynucleotide encoding the serine recombinase is or comprises mRNA.

In some embodiments, a human cell is or comprises: an osteoblast, a chondrocyte, an adipocyte, a skeletal muscle cell, a cardiac muscle cell, a neuron, an astrocyte, an oligodendrocyte, a Schwann cell, a retinal cell, a corneal cell, a skin cell, a monocyte, a macrophage, a neutrophil, a basophil, an eosinophil, an erythrocyte, a megakaryocyte, a dendritic cell, a T-lymphocyte, a B-lymphocyte, an NK-cell, a gastric cell, an intestinal cell, a smooth muscle cell, a vascular cell, a bladder cell, a pancreatic alpha cell, a pancreatic beta cell, a pancreatic delta cell, a liver cell (e.g., a hepatocyte, a hepatic stellate cell, a Kupffer cell, or a liver sinusoidal endothelial cell), a renal cell, an adrenal cell, a lung cell, a mesenchymal stem cell, a hematopoietic stem cell, a hematopoietic progenitor cell, a neuronal stem cell, a retinal stem cell, a cardiac muscle stem cell, a skeletal muscle stem cell, an adipose tissue derived stem cell, a chondrogenic stem cell, a liver stem cell, a kidney stem cell, a pancreatic stem cell, an embryonic stem cell, an induced pluripotent stem cell, or a fate-converted stem or progenitor cell.

In another aspect, the present disclosure provides a transgenic cell (e.g., a human cell) obtained by a method of the present disclosure. In some embodiments, a transgenic cell (e.g., a human cell) is obtained by culturing a transgenic cell (e.g., a human cell) of the present disclosure (e.g., obtained by a method of the present disclosure).

In another aspect, the present disclosure provides methods for obtaining integration of an exogenous nucleic acid (e.g., exogenous DNA) comprising a nucleic acid sequence of interest and a first attachment site into a genome (e.g., a human genome) comprising a second attachment site, the method comprising: contacting the first attachment site with the second attachment site in the presence of a serine recombinase, wherein the contacting step results in recombination between the first and second attachment sites, and wherein recombination between the first and second attachment sites results in integration of the exogenous nucleic acid (e.g., exogenous DNA) into the genome (e.g., human genome).

In some embodiments, a first attachment site is or comprises a donor attachment (attD) site. In some embodiments, an attD site comprises an attB sequence or an attP sequence. In some embodiments, a first attachment site comprises a nucleic acid sequence at least 50% identical to an attB or attP sequence selected from Table 1. In some embodiments, a first attachment site comprises a nucleic acid sequence at least 50% identical to an attB or attP sequence selected from Table 2.

In some embodiments, a second attachment site is or comprises an acceptor attachment (attA) site. In some embodiments, an attA site comprises an attB sequence, an attP sequence, or an attH sequence. In some embodiments, a second attachment site comprises a nucleic acid sequence at least 50% identical to: an attB sequence selected from Table 1, an attP sequence selected from Table 1, or an attH sequence selected from Table 1. In some embodiments, a second attachment site comprises a nucleic acid sequence at least 50% identical to: an attB sequence selected from Table 2, an attP sequence selected from Table 2, or an attH sequence selected from Table 2. In some embodiments, a second attachment site comprises a nucleic acid sequence at least 50% identical to: an attB sequence selected from Table 3, an attP sequence selected from Table 3, or an attH sequence selected from Table 3.

In some embodiments, a serine recombinase comprises an amino acid sequence at least 80% identical to a serine recombinase sequence selected from Table 1. In some embodiments, a serine recombinase comprises an amino acid sequence at least 80% identical to a serine recombinase sequence selected from Table 2. In some embodiments, a serine recombinase comprises an amino acid sequence at least 80% identical to a serine recombinase sequence selected from Table 3.

In some embodiments, a serine recombinase is a recombinase selected from cluster 1 as identified in Table 1. In some embodiments, a serine recombinase is a recombinase selected from cluster 2 as identified in Table 1. In some embodiments, a serine recombinase is a recombinase selected from cluster 3 as identified in Table 1. In some embodiments, a serine recombinase is a recombinase selected from cluster 4 as identified in Table 1. In some embodiments, a serine recombinase is a recombinase selected from cluster 5 as identified in Table 1. In some embodiments, a serine recombinase is a recombinase selected from cluster 6 as identified in Table 1. In some embodiments, a serine recombinase is a recombinase selected from cluster 7 as identified in Table 1. In some embodiments, a serine recombinase is a recombinase selected from cluster 8 as identified in Table 1. In some embodiments, a serine recombinase is a recombinase selected from cluster 9 as identified in Table 1. In some embodiments, a serine recombinase is a recombinase selected from cluster 10 as identified in Table 1. In some embodiments, a serine recombinase is a recombinase selected from cluster 11 as identified in Table 1. In some embodiments, a serine recombinase is a recombinase selected from cluster 12 as identified in Table 1. In some embodiments, a serine recombinase is a recombinase selected from cluster 13 as identified in Table 1. In some embodiments, a serine recombinase is a recombinase selected from cluster 14 as identified in Table 1. In some embodiments, a serine recombinase is a recombinase selected from cluster 15 as identified in Table 1. In some embodiments, a serine recombinase is a recombinase selected from cluster 16 as identified in Table 1. In some embodiments, a serine recombinase is a recombinase selected from cluster 17 as identified in Table 1. In some embodiments, a serine recombinase is a recombinase selected from cluster 18 as identified in Table 1. In some embodiments, a serine recombinase is a recombinase selected from cluster 19 as identified in Table 1. In some embodiments, a serine recombinase is a recombinase selected from cluster 20 as identified in Table 1. In some embodiments, a serine recombinase is a recombinase selected from cluster 21 as identified in Table 1. In some embodiments, a serine recombinase is a recombinase selected from cluster 22 as identified in Table 1. In some embodiments, a serine recombinase is a recombinase selected from cluster 23 as identified in Table 1. In some embodiments, a serine recombinase is a recombinase selected from cluster 24 as identified in Table 1. In some embodiments, a serine recombinase is a recombinase selected from cluster 25 as identified in Table 1. In some embodiments, a serine recombinase is a recombinase selected from cluster 26 as identified in Table 1. In some embodiments, a serine recombinase is a recombinase selected from cluster 27 as identified in Table 1. In some embodiments, a serine recombinase is a recombinase selected from cluster 28 as identified in Table 1. In some embodiments, a serine recombinase is a recombinase selected from cluster 29 as identified in Table 1. In some embodiments, a serine recombinase is a recombinase selected from cluster 30 as identified in Table 1. In some embodiments, a serine recombinase is a recombinase selected from cluster 31 as identified in Table 1. In some embodiments, a serine recombinase is a recombinase selected from cluster 32 as identified in Table 1. In some embodiments, a serine recombinase is a recombinase selected from cluster 33 as identified in Table 1. In some embodiments, a serine recombinase is a recombinase selected from cluster 34 as identified in Table 1. In some embodiments, a serine recombinase is a recombinase selected from cluster 35 as identified in Table 1. In some embodiments, a serine recombinase is a recombinase selected from cluster 36 as identified in Table 1. In some embodiments, a serine recombinase is a recombinase selected from cluster 37 as identified in Table 1. In some embodiments, a serine recombinase is a recombinase selected from cluster 38 as identified in Table 1. In some embodiments, a serine recombinase is a recombinase selected from cluster 39 as identified in Table 1. In some embodiments, a serine recombinase is a recombinase selected from cluster 40 as identified in Table 1. In some embodiments, a serine recombinase is a recombinase selected from cluster 41 as identified in Table 1. In some embodiments, a serine recombinase is a recombinase selected from cluster 42 as identified in Table 1. In some embodiments, a serine recombinase is a recombinase selected from cluster 43 as identified in Table 1. In some embodiments, a serine recombinase is a recombinase selected from cluster 44 as identified in Table 1. In some embodiments, a serine recombinase is a recombinase selected from cluster 45 as identified in Table 1. In some embodiments, a serine recombinase is a recombinase selected from cluster 46 as identified in Table 1. In some embodiments, a serine recombinase is a recombinase selected from cluster 47 as identified in Table 1. In some embodiments, a serine recombinase is a recombinase selected from cluster 48 as identified in Table 1. In some embodiments, a serine recombinase is a recombinase selected from cluster 49 as identified in Table 1. In some embodiments, a serine recombinase is a recombinase selected from cluster 50 as identified in Table 1. In some embodiments, a serine recombinase is a recombinase selected from cluster 51 as identified in Table 1. In some embodiments, a serine recombinase is a recombinase selected from cluster 52 as identified in Table 1. In some embodiments, a serine recombinase is a recombinase selected from cluster 53 as identified in Table 1. In some embodiments, a serine recombinase is a recombinase selected from cluster 54 as identified in Table 1. In some embodiments, a serine recombinase is a recombinase selected from cluster 55 as identified in Table 1. In some embodiments, a serine recombinase is a recombinase selected from cluster 56 as identified in Table 1. In some embodiments, a serine recombinase is a recombinase selected from cluster 57 as identified in Table 1. In some embodiments, a serine recombinase is a recombinase selected from cluster 58 as identified in Table 1. In some embodiments, a serine recombinase is a recombinase selected from cluster 59 as identified in Table 1. In some embodiments, a serine recombinase is a recombinase selected from cluster 60 as identified in Table 1. In some embodiments, a serine recombinase is a recombinase selected from cluster 61 as identified in Table 1. In some embodiments, a serine recombinase is a recombinase selected from cluster 62 as identified in Table 1. In some embodiments, a serine recombinase is a recombinase selected from cluster 63 as identified in Table 1. In some embodiments, a serine recombinase is a recombinase selected from cluster 64 as identified in Table 1. In some embodiments, a serine recombinase is a recombinase selected from cluster 65 as identified in Table 1. In some embodiments, a serine recombinase is a recombinase selected from cluster 66 as identified in Table 1. In some embodiments, a serine recombinase is a recombinase selected from cluster 67 as identified in Table 1. In some embodiments, a serine recombinase is a recombinase selected from cluster 68 as identified in Table 1. In some embodiments, a serine recombinase is a recombinase selected from cluster 69 as identified in Table 1. In some embodiments, a serine recombinase is a recombinase selected from cluster 70 as identified in Table 1. In some embodiments, a serine recombinase is a recombinase selected from cluster 71 as identified in Table 1. In some embodiments, a serine recombinase is a recombinase selected from cluster 72 as identified in Table 1. In some embodiments, a serine recombinase is a recombinase selected from cluster 73 as identified in Table 1. In some embodiments, a serine recombinase is a recombinase selected from cluster 74 as identified in Table 1. In some embodiments, a serine recombinase is a recombinase selected from cluster 75 as identified in Table 1. In some embodiments, a serine recombinase is a recombinase selected from cluster 76 as identified in Table 1. In some embodiments, a serine recombinase is a recombinase selected from cluster 77 as identified in Table 1. In some embodiments, a serine recombinase is a recombinase selected from cluster 78 as identified in Table 1. In some embodiments, a serine recombinase is a recombinase selected from cluster 79 as identified in Table 1. In some embodiments, a serine recombinase is a recombinase selected from cluster 80 as identified in Table 1. In some embodiments, a serine recombinase is a recombinase selected from cluster 81 as identified in Table 1. In some embodiments, a serine recombinase is a recombinase selected from cluster 82 as identified in Table 1. In some embodiments, a serine recombinase is a recombinase selected from cluster 83 as identified in Table 1. In some embodiments, a serine recombinase is a recombinase selected from cluster 84 as identified in Table 1. In some embodiments, a serine recombinase is a recombinase selected from cluster 85 as identified in Table 1. In some embodiments, a serine recombinase is a recombinase selected from cluster 86 as identified in Table 1. In some embodiments, a serine recombinase is a recombinase selected from cluster 87 as identified in Table 1. In some embodiments, a serine recombinase is a recombinase selected from cluster 88 as identified in Table 1. In some embodiments, a serine recombinase is a recombinase selected from cluster 89 as identified in Table 1. In some embodiments, a serine recombinase is a recombinase selected from cluster 90 as identified in Table 1. In some embodiments, a serine recombinase is a recombinase selected from cluster 91 as identified in Table 1. In some embodiments, a serine recombinase is a recombinase selected from cluster 92 as identified in Table 1. In some embodiments, a serine recombinase is a recombinase selected from cluster 93 as identified in Table 1. In some embodiments, a serine recombinase is a recombinase selected from cluster 94 as identified in Table 1. In some embodiments, a serine recombinase is a recombinase selected from cluster 95 as identified in Table 1. In some embodiments, a serine recombinase is a recombinase selected from cluster 96 as identified in Table 1. In some embodiments, a serine recombinase is a recombinase selected from cluster 97 as identified in Table 1. In some embodiments, a serine recombinase is a recombinase selected from cluster 98 as identified in Table 1. In some embodiments, a serine recombinase is a recombinase selected from cluster 99 as identified in Table 1. In some embodiments, a serine recombinase is a recombinase selected from cluster 100 as identified in Table 1. In some embodiments, a serine recombinase is a recombinase selected from cluster 101 as identified in Table 1. In some embodiments, a serine recombinase is a recombinase selected from cluster 102 as identified in Table 1. In some embodiments, a serine recombinase is a recombinase selected from cluster 103 as identified in Table 1. In some embodiments, a serine recombinase is a recombinase selected from cluster 104 as identified in Table 1. In some embodiments, a serine recombinase is a recombinase selected from cluster 105 as identified in Table 1. In some embodiments, a serine recombinase is a recombinase selected from cluster 106 as identified in Table 1. In some embodiments, a serine recombinase is a recombinase selected from cluster 107 as identified in Table 1. In some embodiments, a serine recombinase is a recombinase selected from cluster 108 as identified in Table 1. In some embodiments, a serine recombinase is a recombinase selected from cluster 109 as identified in Table 1. In some embodiments, a serine recombinase is a recombinase selected from cluster 110 as identified in Table 1. In some embodiments, a serine recombinase is a recombinase selected from cluster 111 as identified in Table 1. In some embodiments, a serine recombinase is a recombinase selected from cluster 112 as identified in Table 1. In some embodiments, a serine recombinase is a recombinase selected from cluster 113 as identified in Table 1. In some embodiments, a serine recombinase is a recombinase selected from cluster 114 as identified in Table 1. In some embodiments, a serine recombinase is a recombinase selected from cluster 115 as identified in Table 1. In some embodiments, a serine recombinase is a recombinase selected from cluster 116 as identified in Table 1. In some embodiments, a serine recombinase is a recombinase selected from cluster 117 as identified in Table 1. In some embodiments, a serine recombinase is a recombinase selected from cluster 118 as identified in Table 1. In some embodiments, a serine recombinase is a recombinase selected from cluster 119 as identified in Table 1. In some embodiments, a serine recombinase is a recombinase selected from cluster 120 as identified in Table 1. In some embodiments, a serine recombinase is a recombinase selected from cluster 121 as identified in Table 1. In some embodiments, a serine recombinase is a recombinase selected from cluster 122 as identified in Table 1. In some embodiments, a serine recombinase is a recombinase selected from cluster 123 as identified in Table 1. In some embodiments, a serine recombinase is a recombinase selected from cluster 124 as identified in Table 1. In some embodiments, a serine recombinase is a recombinase selected from cluster 125 as identified in Table 1. In some embodiments, a serine recombinase is a recombinase selected from cluster 126 as identified in Table 1. In some embodiments, a serine recombinase is a recombinase selected from cluster 127 as identified in Table 1. In some embodiments, a serine recombinase is a recombinase selected from cluster 128 as identified in Table 1. In some embodiments, a serine recombinase is a recombinase selected from cluster 129 as identified in Table 1. In some embodiments, a serine recombinase is a recombinase selected from cluster 130 as identified in Table 1. In some embodiments, a serine recombinase is a recombinase selected from cluster 131 as identified in Table 1. In some embodiments, a serine recombinase is a recombinase selected from cluster 132 as identified in Table 1. In some embodiments, a serine recombinase is a recombinase selected from cluster 133 as identified in Table 1. In some embodiments, a serine recombinase is a recombinase selected from cluster 134 as identified in Table 1. In some embodiments, a serine recombinase is a recombinase selected from cluster 135 as identified in Table 1. In some embodiments, a serine recombinase is a recombinase selected from cluster 136 as identified in Table 1. In some embodiments, a serine recombinase is a recombinase selected from cluster 137 as identified in Table 1. In some embodiments, a serine recombinase is a recombinase selected from cluster 138 as identified in Table 1. In some embodiments, a serine recombinase is a recombinase selected from cluster 139 as identified in Table 1. In some embodiments, a serine recombinase is a recombinase selected from cluster 140 as identified in Table 1. In some embodiments, a serine recombinase is a recombinase selected from cluster 141 as identified in Table 1. In some embodiments, a serine recombinase is a recombinase selected from cluster 142 as identified in Table 1. In some embodiments, a serine recombinase is a recombinase selected from cluster 143 as identified in Table 1. In some embodiments, a serine recombinase is a recombinase selected from cluster 144 as identified in Table 1. In some embodiments, a serine recombinase is a recombinase selected from cluster 145 as identified in Table 1. In some embodiments, a serine recombinase is a recombinase selected from cluster 146 as identified in Table 1. In some embodiments, a serine recombinase is a recombinase selected from cluster 147 as identified in Table 1. In some embodiments, a serine recombinase is a recombinase selected from cluster 148 as identified in Table 1. In some embodiments, a serine recombinase is a recombinase selected from cluster 149 as identified in Table 1. In some embodiments, a serine recombinase is a recombinase selected from cluster 150 as identified in Table 1. In some embodiments, a serine recombinase is a recombinase selected from cluster 151 as identified in Table 1. In some embodiments, a serine recombinase is a recombinase selected from cluster 152 as identified in Table 1. In some embodiments, a serine recombinase is a recombinase selected from cluster 153 as identified in Table 1. In some embodiments, a serine recombinase is a recombinase selected from cluster 154 as identified in Table 1. In some embodiments, a serine recombinase is a recombinase selected from cluster 155 as identified in Table 1. In some embodiments, a serine recombinase is a recombinase selected from cluster 156 as identified in Table 1. In some embodiments, a serine recombinase is a recombinase selected from cluster 157 as identified in Table 1. In some embodiments, a serine recombinase is a recombinase selected from cluster 158 as identified in Table 1. In some embodiments, a serine recombinase is a recombinase selected from cluster 159 as identified in Table 1.

In some embodiments, a serine recombinase, a first attachment site, and a second attachment site comprise sequences at least 80% identical to sequences that have the same system ID in Table 1. In some embodiments, a serine recombinase, a first attachment site, and a second attachment site comprise sequences at least 80% identical to sequences that have the same system ID in Table 2. In some embodiments, a serine recombinase, a first attachment site, and a second attachment site comprise sequences at least 80% identical to sequences that have the same system ID in Table 3.

In another aspect, the present disclosure provides a system for integrating an exogenous nucleic acid (e.g., exogenous DNA) comprising a nucleic acid sequence of interest into a genome (e.g., human genome), the system comprising: an exogenous nucleic acid (e.g., exogenous DNA) comprising a nucleic acid sequence of interest and a first attachment site, and a serine recombinase or a polynucleotide encoding the serine recombinase.

In some embodiments, a system comprises a polynucleotide encoding a serine recombinase and the polynucleotide comprises mRNA. In some embodiments, a system comprises a polynucleotide encoding the serine recombinase and the polynucleotide comprises DNA.

In some embodiments, exogenous nucleic acid (e.g., exogenous DNA) is or comprises a plasmid, a nanoplasmid, a mini-circle, or doggybone DNA (dbDNA).

In some embodiments, a system comprises a lipid nanoparticle (LNP), an adeno-associated virus (AAV), a lentivirus, a virus-like particle (VLP), an exosome, a cationic nanoparticle, or a dendrimer.

In some embodiments, a first attachment site is or comprises a donor attachment (attD) site. In some embodiments, an attD site comprises an attB sequence or an attP sequence. In some embodiments, a first attachment site comprises a nucleic acid sequence at least 50% identical to an attB or attP sequence selected from Table 1. In some embodiments, a first attachment site comprises a nucleic acid sequence at least 50% identical to an attB or attP sequence selected from Table 2. In some embodiments, a first attachment site comprises a nucleic acid sequence at least 50% identical to an attB or attP sequence selected from Table 3.

In some embodiments, a genome (e.g., a human genome) comprises a second attachment site. In some embodiments, a second attachment site is or comprises an acceptor attachment (attA) site. In some embodiments, an attA site comprises an attB sequence, an attP sequence, or an attH sequence. In some embodiments, a second attachment site comprises a nucleic acid sequence at least 50% identical to: an attB sequence selected from Table 1, an attP sequence selected from Table 1, or an attH sequence selected from Table 1. In some embodiments, a second attachment site comprises a nucleic acid sequence at least 50% identical to: an attB sequence selected from Table 2, an attP sequence selected from Table 2, or an attH sequence selected from Table 2. In some embodiments, a second attachment site comprises a nucleic acid sequence at least 50% identical to: an attB sequence selected from Table 3, an attP sequence selected from Table 3, or an attH sequence selected from Table 3.

In some embodiments, a serine recombinase comprises an amino acid sequence at least 80% identical to a sequence selected from Table 1. In some embodiments, a serine recombinase comprises an amino acid sequence at least 80% identical to a sequence selected from Table 2. In some embodiments, a serine recombinase comprises an amino acid sequence at least 80% identical to a sequence selected from Table 3.

In some embodiments, a serine recombinase is a recombinase selected from cluster 1 as identified in Table 1. In some embodiments, a serine recombinase is a recombinase selected from cluster 2 as identified in Table 1. In some embodiments, a serine recombinase is a recombinase selected from cluster 3 as identified in Table 1. In some embodiments, a serine recombinase is a recombinase selected from cluster 4 as identified in Table 1. In some embodiments, a serine recombinase is a recombinase selected from cluster 5 as identified in Table 1. In some embodiments, a serine recombinase is a recombinase selected from cluster 6 as identified in Table 1. In some embodiments, a serine recombinase is a recombinase selected from cluster 7 as identified in Table 1. In some embodiments, a serine recombinase is a recombinase selected from cluster 8 as identified in Table 1. In some embodiments, a serine recombinase is a recombinase selected from cluster 9 as identified in Table 1. In some embodiments, a serine recombinase is a recombinase selected from cluster 10 as identified in Table 1. In some embodiments, a serine recombinase is a recombinase selected from cluster 11 as identified in Table 1. In some embodiments, a serine recombinase is a recombinase selected from cluster 12 as identified in Table 1. In some embodiments, a serine recombinase is a recombinase selected from cluster 13 as identified in Table 1. In some embodiments, a serine recombinase is a recombinase selected from cluster 14 as identified in Table 1. In some embodiments, a serine recombinase is a recombinase selected from cluster 15 as identified in Table 1. In some embodiments, a serine recombinase is a recombinase selected from cluster 16 as identified in Table 1. In some embodiments, a serine recombinase is a recombinase selected from cluster 17 as identified in Table 1. In some embodiments, a serine recombinase is a recombinase selected from cluster 18 as identified in Table 1. In some embodiments, a serine recombinase is a recombinase selected from cluster 19 as identified in Table 1. In some embodiments, a serine recombinase is a recombinase selected from cluster 20 as identified in Table 1. In some embodiments, a serine recombinase is a recombinase selected from cluster 21 as identified in Table 1. In some embodiments, a serine recombinase is a recombinase selected from cluster 22 as identified in Table 1. In some embodiments, a serine recombinase is a recombinase selected from cluster 23 as identified in Table 1. In some embodiments, a serine recombinase is a recombinase selected from cluster 24 as identified in Table 1. In some embodiments, a serine recombinase is a recombinase selected from cluster 25 as identified in Table 1. In some embodiments, a serine recombinase is a recombinase selected from cluster 26 as identified in Table 1. In some embodiments, a serine recombinase is a recombinase selected from cluster 27 as identified in Table 1. In some embodiments, a serine recombinase is a recombinase selected from cluster 28 as identified in Table 1. In some embodiments, a serine recombinase is a recombinase selected from cluster 29 as identified in Table 1. In some embodiments, a serine recombinase is a recombinase selected from cluster 30 as identified in Table 1. In some embodiments, a serine recombinase is a recombinase selected from cluster 31 as identified in Table 1. In some embodiments, a serine recombinase is a recombinase selected from cluster 32 as identified in Table 1. In some embodiments, a serine recombinase is a recombinase selected from cluster 33 as identified in Table 1. In some embodiments, a serine recombinase is a recombinase selected from cluster 34 as identified in Table 1. In some embodiments, a serine recombinase is a recombinase selected from cluster 35 as identified in Table 1. In some embodiments, a serine recombinase is a recombinase selected from cluster 36 as identified in Table 1. In some embodiments, a serine recombinase is a recombinase selected from cluster 37 as identified in Table 1. In some embodiments, a serine recombinase is a recombinase selected from cluster 38 as identified in Table 1. In some embodiments, a serine recombinase is a recombinase selected from cluster 39 as identified in Table 1. In some embodiments, a serine recombinase is a recombinase selected from cluster 40 as identified in Table 1. In some embodiments, a serine recombinase is a recombinase selected from cluster 41 as identified in Table 1. In some embodiments, a serine recombinase is a recombinase selected from cluster 42 as identified in Table 1. In some embodiments, a serine recombinase is a recombinase selected from cluster 43 as identified in Table 1. In some embodiments, a serine recombinase is a recombinase selected from cluster 44 as identified in Table 1. In some embodiments, a serine recombinase is a recombinase selected from cluster 45 as identified in Table 1. In some embodiments, a serine recombinase is a recombinase selected from cluster 46 as identified in Table 1. In some embodiments, a serine recombinase is a recombinase selected from cluster 47 as identified in Table 1. In some embodiments, a serine recombinase is a recombinase selected from cluster 48 as identified in Table 1. In some embodiments, a serine recombinase is a recombinase selected from cluster 49 as identified in Table 1. In some embodiments, a serine recombinase is a recombinase selected from cluster 50 as identified in Table 1. In some embodiments, a serine recombinase is a recombinase selected from cluster 51 as identified in Table 1. In some embodiments, a serine recombinase is a recombinase selected from cluster 52 as identified in Table 1. In some embodiments, a serine recombinase is a recombinase selected from cluster 53 as identified in Table 1. In some embodiments, a serine recombinase is a recombinase selected from cluster 54 as identified in Table 1. In some embodiments, a serine recombinase is a recombinase selected from cluster 55 as identified in Table 1. In some embodiments, a serine recombinase is a recombinase selected from cluster 56 as identified in Table 1. In some embodiments, a serine recombinase is a recombinase selected from cluster 57 as identified in Table 1. In some embodiments, a serine recombinase is a recombinase selected from cluster 58 as identified in Table 1. In some embodiments, a serine recombinase is a recombinase selected from cluster 59 as identified in Table 1. In some embodiments, a serine recombinase is a recombinase selected from cluster 60 as identified in Table 1. In some embodiments, a serine recombinase is a recombinase selected from cluster 61 as identified in Table 1. In some embodiments, a serine recombinase is a recombinase selected from cluster 62 as identified in Table 1. In some embodiments, a serine recombinase is a recombinase selected from cluster 63 as identified in Table 1. In some embodiments, a serine recombinase is a recombinase selected from cluster 64 as identified in Table 1. In some embodiments, a serine recombinase is a recombinase selected from cluster 65 as identified in Table 1. In some embodiments, a serine recombinase is a recombinase selected from cluster 66 as identified in Table 1. In some embodiments, a serine recombinase is a recombinase selected from cluster 67 as identified in Table 1. In some embodiments, a serine recombinase is a recombinase selected from cluster 68 as identified in Table 1. In some embodiments, a serine recombinase is a recombinase selected from cluster 69 as identified in Table 1. In some embodiments, a serine recombinase is a recombinase selected from cluster 70 as identified in Table 1. In some embodiments, a serine recombinase is a recombinase selected from cluster 71 as identified in Table 1. In some embodiments, a serine recombinase is a recombinase selected from cluster 72 as identified in Table 1. In some embodiments, a serine recombinase is a recombinase selected from cluster 73 as identified in Table 1. In some embodiments, a serine recombinase is a recombinase selected from cluster 74 as identified in Table 1. In some embodiments, a serine recombinase is a recombinase selected from cluster 75 as identified in Table 1. In some embodiments, a serine recombinase is a recombinase selected from cluster 76 as identified in Table 1. In some embodiments, a serine recombinase is a recombinase selected from cluster 77 as identified in Table 1. In some embodiments, a serine recombinase is a recombinase selected from cluster 78 as identified in Table 1. In some embodiments, a serine recombinase is a recombinase selected from cluster 79 as identified in Table 1. In some embodiments, a serine recombinase is a recombinase selected from cluster 80 as identified in Table 1. In some embodiments, a serine recombinase is a recombinase selected from cluster 81 as identified in Table 1. In some embodiments, a serine recombinase is a recombinase selected from cluster 82 as identified in Table 1. In some embodiments, a serine recombinase is a recombinase selected from cluster 83 as identified in Table 1. In some embodiments, a serine recombinase is a recombinase selected from cluster 84 as identified in Table 1. In some embodiments, a serine recombinase is a recombinase selected from cluster 85 as identified in Table 1. In some embodiments, a serine recombinase is a recombinase selected from cluster 86 as identified in Table 1. In some embodiments, a serine recombinase is a recombinase selected from cluster 87 as identified in Table 1. In some embodiments, a serine recombinase is a recombinase selected from cluster 88 as identified in Table 1. In some embodiments, a serine recombinase is a recombinase selected from cluster 89 as identified in Table 1. In some embodiments, a serine recombinase is a recombinase selected from cluster 90 as identified in Table 1. In some embodiments, a serine recombinase is a recombinase selected from cluster 91 as identified in Table 1. In some embodiments, a serine recombinase is a recombinase selected from cluster 92 as identified in Table 1. In some embodiments, a serine recombinase is a recombinase selected from cluster 93 as identified in Table 1. In some embodiments, a serine recombinase is a recombinase selected from cluster 94 as identified in Table 1. In some embodiments, a serine recombinase is a recombinase selected from cluster 95 as identified in Table 1. In some embodiments, a serine recombinase is a recombinase selected from cluster 96 as identified in Table 1. In some embodiments, a serine recombinase is a recombinase selected from cluster 97 as identified in Table 1. In some embodiments, a serine recombinase is a recombinase selected from cluster 98 as identified in Table 1. In some embodiments, a serine recombinase is a recombinase selected from cluster 99 as identified in Table 1. In some embodiments, a serine recombinase is a recombinase selected from cluster 100 as identified in Table 1. In some embodiments, a serine recombinase is a recombinase selected from cluster 101 as identified in Table 1. In some embodiments, a serine recombinase is a recombinase selected from cluster 102 as identified in Table 1. In some embodiments, a serine recombinase is a recombinase selected from cluster 103 as identified in Table 1. In some embodiments, a serine recombinase is a recombinase selected from cluster 104 as identified in Table 1. In some embodiments, a serine recombinase is a recombinase selected from cluster 105 as identified in Table 1. In some embodiments, a serine recombinase is a recombinase selected from cluster 106 as identified in Table 1. In some embodiments, a serine recombinase is a recombinase selected from cluster 107 as identified in Table 1. In some embodiments, a serine recombinase is a recombinase selected from cluster 108 as identified in Table 1. In some embodiments, a serine recombinase is a recombinase selected from cluster 109 as identified in Table 1. In some embodiments, a serine recombinase is a recombinase selected from cluster 110 as identified in Table 1. In some embodiments, a serine recombinase is a recombinase selected from cluster 111 as identified in Table 1. In some embodiments, a serine recombinase is a recombinase selected from cluster 112 as identified in Table 1. In some embodiments, a serine recombinase is a recombinase selected from cluster 113 as identified in Table 1. In some embodiments, a serine recombinase is a recombinase selected from cluster 114 as identified in Table 1. In some embodiments, a serine recombinase is a recombinase selected from cluster 115 as identified in Table 1. In some embodiments, a serine recombinase is a recombinase selected from cluster 116 as identified in Table 1. In some embodiments, a serine recombinase is a recombinase selected from cluster 117 as identified in Table 1. In some embodiments, a serine recombinase is a recombinase selected from cluster 118 as identified in Table 1. In some embodiments, a serine recombinase is a recombinase selected from cluster 119 as identified in Table 1. In some embodiments, a serine recombinase is a recombinase selected from cluster 120 as identified in Table 1. In some embodiments, a serine recombinase is a recombinase selected from cluster 121 as identified in Table 1. In some embodiments, a serine recombinase is a recombinase selected from cluster 122 as identified in Table 1. In some embodiments, a serine recombinase is a recombinase selected from cluster 123 as identified in Table 1. In some embodiments, a serine recombinase is a recombinase selected from cluster 124 as identified in Table 1. In some embodiments, a serine recombinase is a recombinase selected from cluster 125 as identified in Table 1. In some embodiments, a serine recombinase is a recombinase selected from cluster 126 as identified in Table 1. In some embodiments, a serine recombinase is a recombinase selected from cluster 127 as identified in Table 1. In some embodiments, a serine recombinase is a recombinase selected from cluster 128 as identified in Table 1. In some embodiments, a serine recombinase is a recombinase selected from cluster 129 as identified in Table 1. In some embodiments, a serine recombinase is a recombinase selected from cluster 130 as identified in Table 1. In some embodiments, a serine recombinase is a recombinase selected from cluster 131 as identified in Table 1. In some embodiments, a serine recombinase is a recombinase selected from cluster 132 as identified in Table 1. In some embodiments, a serine recombinase is a recombinase selected from cluster 133 as identified in Table 1. In some embodiments, a serine recombinase is a recombinase selected from cluster 134 as identified in Table 1. In some embodiments, a serine recombinase is a recombinase selected from cluster 135 as identified in Table 1. In some embodiments, a serine recombinase is a recombinase selected from cluster 136 as identified in Table 1. In some embodiments, a serine recombinase is a recombinase selected from cluster 137 as identified in Table 1. In some embodiments, a serine recombinase is a recombinase selected from cluster 138 as identified in Table 1. In some embodiments, a serine recombinase is a recombinase selected from cluster 139 as identified in Table 1. In some embodiments, a serine recombinase is a recombinase selected from cluster 140 as identified in Table 1. In some embodiments, a serine recombinase is a recombinase selected from cluster 141 as identified in Table 1. In some embodiments, a serine recombinase is a recombinase selected from cluster 142 as identified in Table 1. In some embodiments, a serine recombinase is a recombinase selected from cluster 143 as identified in Table 1. In some embodiments, a serine recombinase is a recombinase selected from cluster 144 as identified in Table 1. In some embodiments, a serine recombinase is a recombinase selected from cluster 145 as identified in Table 1. In some embodiments, a serine recombinase is a recombinase selected from cluster 146 as identified in Table 1. In some embodiments, a serine recombinase is a recombinase selected from cluster 147 as identified in Table 1. In some embodiments, a serine recombinase is a recombinase selected from cluster 148 as identified in Table 1. In some embodiments, a serine recombinase is a recombinase selected from cluster 149 as identified in Table 1. In some embodiments, a serine recombinase is a recombinase selected from cluster 150 as identified in Table 1. In some embodiments, a serine recombinase is a recombinase selected from cluster 151 as identified in Table 1. In some embodiments, a serine recombinase is a recombinase selected from cluster 152 as identified in Table 1. In some embodiments, a serine recombinase is a recombinase selected from cluster 153 as identified in Table 1. In some embodiments, a serine recombinase is a recombinase selected from cluster 154 as identified in Table 1. In some embodiments, a serine recombinase is a recombinase selected from cluster 155 as identified in Table 1. In some embodiments, a serine recombinase is a recombinase selected from cluster 156 as identified in Table 1. In some embodiments, a serine recombinase is a recombinase selected from cluster 157 as identified in Table 1. In some embodiments, a serine recombinase is a recombinase selected from cluster 158 as identified in Table 1. In some embodiments, a serine recombinase is a recombinase selected from cluster 159 as identified in Table 1.

In some embodiments, a serine recombinase, a first attachment site, and a second attachment site comprise sequences at least 80% identical to sequences that have the same system ID in Table 1. In some embodiments, a serine recombinase, a first attachment site, and a second attachment site comprise sequences at least 80% identical to sequences that have the same system ID in Table 2. In some embodiments, a serine recombinase, a first attachment site, and a second attachment site comprise sequences at least 80% identical to sequences that have the same system ID in Table 3.

In another aspect, the present disclosure provides a transgenic human cell comprising a system of the present disclosure.

In another aspect, the present disclosure provides a serine recombinase (e.g., an isolated serine recombinase) comprising an amino acid sequence at least 80% identical to a sequence selected from Table 1. In some embodiments, a serine recombinase (e.g., an isolated serine recombinase) comprises an amino acid sequence at least 80% identical to a sequence selected from Table 2. In some embodiments, a serine recombinase (e.g., an isolated serine recombinase) comprises an amino acid sequence at least 80% identical to a sequence selected from Table 3. In some embodiments, a serine recombinase (e.g., an isolated serine recombinase) is fused to one or more nuclear localization signals (NLS). In some embodiments, a nuclear localization signal is fused to the N-terminal of a serine recombinase (e.g., an isolated serine recombinase). In some embodiments, a nuclear localization signal is fused to the C-terminal of a serine recombinase (e.g., an isolated serine recombinase).

In another aspect, the present disclosure provides a nucleic acid (e.g., an isolated nucleic acid) comprising a polynucleotide encoding a serine recombinase of the present disclosure. In another aspect, the present disclosure provides an expression vector comprising a nucleic acid of the present disclosure. In some embodiments, an expression vector comprises a polynucleotide operably linked to a promoter that is active in a human cell. In another aspect, the present disclosure provides a cell (e.g., a transgenic cell, e.g., a transgenic human cell) comprising a serine recombinase of the present disclosure, a nucleic acid of the present disclosure, or an expression vector of the present disclosure. In another aspect, the present disclosure provides a method of treating a disease in a subject in need thereof, the method comprising administering to the subject a system of the present disclosure, a serine recombinase of the present disclosure, a nucleic acid of the present disclosure, an expression vector of the present disclosure, or a cell of the present disclosure.

### BRIEF DESCRIPTION OF THE DRAWING

FIG. 1 shows an exemplary illustration of recombinase-mediated integration between an integrative vector and a human genome. In this illustration, the pair of attachment sites involved in the recombination event are present in the human genome (attH) and in the integrative vector (attD).

FIG. 2 shows an exemplary pair of attP and attB sequences (SEQ ID NO: 2 and SEQ ID NO: 3, respectively). The pair of attachment site sequences comprise pairs of binding regions flanking the central dinucleotide (e.g., TT). The pair of attachment site sequences comprise a pair of recombinase domain (RD) binding regions directly 5′ and 3′ of the central dinucleotide. The pair of attachment site sequences also comprise a pair of zinc ribbon domain (ZD) binding regions 5′ and 3′ of the RD binding regions. The attP attachment site sequence comprises linkers between the RD binding regions and the ZD binding regions.

FIG. 3 shows an exemplary illustration of a plasmid recombination assay. In this illustration, an attB-LSR plasmid and an attP-mCherry plasmid are co-transfected in a cellular system (e.g., HEK293T cells). Upon successful recombination, the mCherry fluorescent protein is capable of expression in the cellular system.

FIGS. 4A-B are exemplary graphs demonstrating percent recombination (FIG. 4A) relative to Bxb1 control and mean fluorescence intensity (MFI, FIG. 4B) as measured by digital droplet PCR (ddPCR). Fluorescent data in FIG. 4B was normalized by dividing the MFI of the recombination group (co-transfection of attB-LSR plasmid and attP-mCherry plasmid; “LSR”) by the MFI of the promoterless attP-mCherry only group (“attP only”) to determine fold increase in mCherry fluorescence caused by promoter-swapping.

FIG. 5 is an exemplary schematic demonstrating clustering and assaying of novel large serine recombinases (LSRs) using methods disclosed in Example 2.

FIGS. 6A-C show an exemplary illustration of a recombination assay (FIG. 6A), an exemplary graph demonstrating percent recombination via the activity of barcoded LSR cluster representatives on barcoded attB plasmids as determined by next generation sequencing (NGS) readout for recombined barcodes (FIG. 6B, with control recombinase Bxb1 shown as “160”), and an exemplary graph demonstrating barcode reads relative to corrected reads for AttR (FIG. 6C).

FIGS. 7A-B show exemplary illustrations for measuring genomic integration using the UDiTaS protocol as disclosed in Example 2. As shown in FIG. 7A, the UDiTas reporter plasmid would target its own attD site for integration into the human genome. As shown in FIG. 7B, when LSR integration occurs, amplicons that are half attD site and half human genome are generated, whereas when random integration occurs, amplicons containing the whole attD site are generated.

FIGS. 8A-B are exemplary graphs demonstrating barcode read count for two separate experiments, each involving three separate groups. FIG. 8A shows unique molecular identifier (UMI) counts across two experiments (first experiment (REQ3707-001): top three graphs and second experiment (REQ3718-001): bottom three graphs). The top graph of each trio (graphs 1 and 4 from the top) represents LSR group 1 (“specific” targeting pool), the middle graph of each trio (graphs 2 and 5 from the top) represents LSR group 2 (“multi-targeting” pool), and the bottom graph of each trio (graphs 3 and 6 from the top) represents the control group. FIG. 8B shows a UMI count comparison across both experiments, denoted Experiment 1 and Experiment 2, of different LSR cluster groups.

FIGS. 9A-B are exemplary graphs demonstrating genomic integration across LSR clusters. FIG. 9A shows a graph comparing number of landing sites across UMI counts for the different LSR clusters. FIG. 9B highlights two outliers (clusters 16 and 85) which both demonstrated a high UMI count with a low number of landing sites.

FIG. 10 is a graph depicting number of landing sites and UMI counts for the different LSR clusters as determined by the pooled genomic integration assay (described in Example 2) with an overlaid heatmap corresponding to activity of the LSR cluster in the pooled plasmid recombination assay (PRA; as described in Example 2). Two LSR clusters (clusters 112 and 136) were noted in the right set of graphs for their targeting profile at various loci.

FIG. 11 is a graph demonstrating percent of UMI read counts across the LSR clusters disclosed gated within the top five landing sites for integration (as a measure of LSR specificity) as well as total UMI read counts (as measure of LSR recombination activity).

### DEFINITIONS

Approximately: as used herein, “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context.

Cognate: as used herein, “cognate” refers to the attribute of a serine recombinase to recognize specific attP and attB attachment sites. It is understood in the art that given the thousands of possible attB attachment sites for any given serine recombinase and attP attachment site to recombine, only a select few will undergo actual recombination. As such, these attB sites are ‘cognate’ with their associated attP site and serine recombinase.

Enhancer: as used herein, “enhancer” refers to a short region of DNA that can be bound by proteins to increase the likelihood for transcription of a particular gene. These bound proteins are usually referred to as transcription factors. Enhancers can be located up to 1 Mbp upstream or downstream from the gene.

Expression Vector: as used herein, “expression vector” refers to a vector, e.g., a nucleic acid delivery vehicle, for example, such as a DNA delivery vehicle, such as a plasmid, nanoplasmid, or doggybone DNA (dbDNA) designed with the capacity to enable expression of a nucleic acid sequence inserted in the vector following transformation into a host. As disclosed herein, an expression vector can encode, for example, a recombinase, or a nucleic acid sequence of interest intended for integration into the genome of a host cell and a recombinase attachment site (e.g., a donor attachment (“attD”) site, as described herein). The inserted nucleic acid sequence is typically under the control of elements such as promoters, initiation control regions, enhancers, and the like. Initiation control regions or promoters are known to those in the art as elements that are useful to drive expression of a nucleic acid of interest in the desired host cell. The expression vector may be RNA, e.g., mRNA, or DNA. In some embodiments, the expression vector can be double-stranded, e.g., a double-stranded DNA plasmid (dsDNA plasmid). In some embodiments, the expression vector can be single-stranded, e.g., a single-stranded DNA plasmid (ssDNA plasmid). In some cases, the expression vector can be linear (e.g., a linear dsDNA plasmid or a linear ssDNA plasmid).

Gene: as used herein, “gene” refers to an assembly of nucleotides that encodes the synthesis of a gene product, either an RNA, a polypeptide, or a protein.

Homologous: as used herein, “homologous” refers to the relationship between proteins that may possess a “common evolutionary origin.” This further includes proteins from superfamilies and homologous proteins from different species. Homologous proteins typically have high percent identity, with variation most often found in redundant codons.

In vitro: as used herein “in vitro” refers to events that occur in an artificial environment, e.g., in a test tube or reaction vessel, in cell culture, etc., rather than within a multi-cellular organism.

In vivo: as used herein, “in vivo” refers to events that occur within a multi-cellular organism, such as a human or a non-human animal.

Nucleic acid: as used herein, the terms “nucleic acid” and “polynucleotide” refer to a polymer of at least three nucleotides. In some embodiments, a nucleic acid comprises DNA. In some embodiments, a nucleic acid comprises RNA, for example, mRNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 1-methyl-pseudouridine, N1-methyl-pseudouridine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro), reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. Nucleic acid sequences provided herein, including, but not limited to those in the sequence listing, are intended to encompass corresponding nucleic acid sequences containing any combination of natural or modified RNA and/or DNA, including, but not limited to, such nucleic acids having modified nucleobases. By way of further example and without limitation, a nucleic acid having the nucleobase sequence “ATCGATCG” encompasses any nucleic acid having such nucleobase sequence, whether modified or unmodified, including, but not limited to, such nucleic acids comprising RNA bases, such as those comprising the sequence “AUCGAUCG” and those comprising some DNA bases and some RNA bases such as “AUCGATCG” and nucleic acids comprising other modified or naturally occurring bases, such as “ATmeCGAUCG,” wherein meC indicates a cytosine base comprising a methyl group at the 5-position.

Percent identity: as used herein, “percent identity” refers to the relationship between two or more polypeptide sequences or two or more polynucleotide sequences as determined by comparing the sequences. “Identity” also means the degree of sequence relatedness between polypeptide or polynucleotide sequences as determined by the match between strings of such sequences. “Identity” also refers to the degree of sequence relatedness between DNA and RNA (e.g., mRNA) polynucleotide sequences as determined by the match between strings of such sequences. “Identity” and “similarity” can be calculated by known methods, including but not limited to those described herein.

Plasmid: as used herein, “plasmid” refers to a genetic structure that can replicate independently of the chromosomes. Plasmids typically exist as small, circular, double-stranded DNA molecules in bacterium. A plasmid carrying a nucleic acid sequence of interest can be circular or linearized prior to delivery into a cell.

Polypeptide: as used herein, “polypeptide” refers to a polymeric compound comprising covalently linked amino acid residues. One or more polypeptides characterized by a stable functional structure are referred to as a “protein.”

Promoter: as used herein, a “promoter” refers to a control region of a nucleic acid at which both initiation and the rate of transcription of downstream DNA is controlled. It is a region whereupon relevant proteins (e.g., RNA polymerase II and transcription factors) bind to initiate transcription of a gene. Resulting transcription results in an RNA molecule (e.g., mRNA). Promoters can be “operably linked” to a nucleic acid sequence. To be “operably linked,” a promoter must be in the correct functional location and orientation relative to the nucleic acid sequence in order for it to regulate said sequence. Promoters can include “constitutive promoters” or “inducible promoters”. A constitutive promoter refers to an unregulated promoter that allows for continual transcription of its associated nucleic acid. An inducible promoter is conditioned in a way to act almost as a “gene switch” whereupon endogenous factors, external stimuli, chemical compounds, or environmental conditions can be artificially controlled to initiate promoter activity.

Recombinase: as used herein, “recombinase” refers to an enzyme capable of catalyzing site-specific recombination events within DNA. Most recombinases fall within two families, tyrosine recombinases and serine recombinases. These families are attributed to the conserved amino acid residue that serves as the nucleophile in the series of transesterification reactions with the DNA strand during recombinase activity. Of particular interest are serine recombinases, which have a specific type of recombination site and a specific mode of activity. Serine recombinases are clustered into three main groups along phylogenetic lines, referred to as (a) large serine recombinases, (b) resolvase/invertases, and (c) IS607-like (Smith & Thorpe, 2002). A serine recombinase may be delivered into a cell as either a protein or as a nucleic acid (e.g., a DNA or mRNA molecule) that encodes the recombinase. A nucleic acid encoding this recombinase may also contain other regulatory components, e.g., suitable promoters, regulators, and/or enhancers. A nucleic acid encoding the recombinase may contain modified or alternative nucleotides and/or other chemical modifications.

Recombination attachment sites: as used herein, “recombination attachment sites” refers to a pair of attachment sites that are recognized by and acted upon by a recombinase. In some embodiments, an attachment site is referred to as “att” or an “att site”. In some embodiments, these sites denote their origin and evolution from bacteriophages, wherein the bacteriophage genome, containing an “attP” site, can integrate into the host bacterial chromosome, containing an “attB site”. In nature, both attB and attP sites are specific for each serine recombinase, such that a particular recombinase mediates DNA recombination between a specific attP site and a specific attB site. These attP and attB sites are not homologous, thus recombination between attB and attP sites results in new attachment sites known as “attL” and “attR”. The reverse excision reaction between these new attL and attR sites does not occur in the absence of a phage-encoded recombination directionality factor (RDF). Attachment sites of the present disclosure may also comprise non-bacterial or phage sequences as described herein, including variants of the natural attB and attP sites (e.g., variants that include different central dinucleotides) and attachment sites in the human genome (“attH”) that are able to recombine with a natural or variant attP or attB site in the presence of the particular recombinase. These attH sites may exist in one or more desired location(s) in the human genome. In some embodiments, an attH site in the human genome can be identical to either an attB or attP site. In some embodiments an attH site can have homology to either an attB or an attP sequence. For example, an attH site with homology to an attB site may recombine with the attP site that normally recombines with the attB site while an attH site with homology to an attP site may recombine with the attB site that normally recombines with the attP site. In these circumstances, the attP/B site that can specifically recombine with an attH site is referred to as an “attD site” (i.e., donor attachment site, e.g., an attachment site in a donor plasmid). Variants of the natural attB and attP sites (e.g., variants that include different central dinucleotides) that can specifically recombine with an attH site are also considered attD sites of the present disclosure.

Target site: as used herein, “target site” describes a location bearing an attachment site (e.g., a cognate attachment site) for an exogenous nucleic acid (e.g., exogenous DNA), such as an exogenous DNA carrying a nucleic acid sequence of interest. For example, a target site may comprise an attB site that will recombine with a cognate attP site of an exogenous nucleic acid (e.g., exogenous DNA) in the presence of the particular recombinase. A target site may also be a site that is homologous but not identical to a bacterial or phage attachment site sequence, but instead be a “human attachment site” (attH site) identified in the human genome that is capable of recombining with the corresponding attB or attP site in the presence of the particular recombinase.

### DETAILED DESCRIPTION

Site-specific recombination involves the specialized movement of genetic elements into and out of non-homologous regions within a genome or between genomes. Mobilization of these genetic elements can occur within a single chromosome or between two different chromosomes, giving rise to variations essential for adaptation and evolution. While abundant among bacteria and viruses, site-specification recombination can still function in heterologous systems, such as mammalian cells, potentially making it a very useful tool for manipulation or engineering of the genome via integration, excision, or inversion events.

A number of challenges currently exist in terms of applying these tools in a human genome context. For one, the ability of DNA integration to occur is governed by the presence of specific attachment sites that are cognate with a recombinase. Problematically, previously identified attachment sites do not exist in the human chromosome. Before recombinase-mediated DNA integration could be performed, the human cell would therefore have to first be engineered by adding attachment sites at desired locations to allow for site-specific recombination to occur. This requirement for an additional step is time-consuming and costly.

The present disclosure provides a number of novel large serine recombinases identified to target a number of novel attachment sites in the human genome. The applications of these novel large serine recombinases allow for genetic integration of large DNA payloads that is highly specific, efficient, and avoids complications of prior methodology.

### Site-Specific Recombinases

Site-specific recombinases recognize two specific sequences present on one or two DNA molecules, catalyzing the cleavage of specific phosphodiester bonds within these two “attachment” sites, and rejoins these broken ends to form recombinants (Olorunniji et al. 2016). This process doesn't require extensive DNA homology, as does homologous recombination (HR), nor does it involve any DNA synthesis or degradation. As such, this form of recombinase-mediated recombination is often referred to as conservative site-specific recombination.

Based on amino acid sequence homology, conservative site-specific recombinases fall into one of two mechanistically different families: tyrosine recombinases and serine recombinases. Each family is named according to the identity of the active nucleophilic amino acid residue responsible for attacking the DNA phosphodiester bonds to create strand breaks, and subsequent formation of a covalent linkage to conserve bond energy for recombination (Olorunniji et al. 2016). While there are a number of features shared by both families, their proteins have diverging sequences and are structurally distinct. Furthermore, both families operate using different recombination mechanisms.

### Tyrosine Recombinase Family

Some of the most well-known recombinases are in the tyrosine recombinase family. Tyrosine recombinases carry out recombination by breaking, exchanging, and rejoining DNA strands two at a time through the formation of a “Holliday junction” or four-way intermediate. Within these Holliday junctions, two of the strands are recombinant whereas the other two strands are non-recombinant. There is a specific amount of separation between breaks in the top and bottom strand of DNA for each tyrosine recombinase system (Olorunniji et al. 2016).

Tyrosine recombinase systems perform diverse programmed DNA rearrangements in bacteria, archaea, viruses, and lower eukaryotes, including integration and excision of DNA, monomerization of chromosome and plasmid multimers, circulation of bacteriophage replication intermediates, resolution of transposition intermediates, inversion-mediated switching of gene expression, and amplification of plasmid copy number. Intriguingly, tyrosine recombinases both structurally and mechanistically are related to Type IB topoisomerases, which include the human topoisomerase (Olorunniji et al. 2016).

A key functional component of tyrosine recombinases is a catalytic domain, which plays a crucial role in DNA sequence recognition, subunit interactions, and regulatory functions. Within the catalytic domain is an active site, which comprises four highly conserved residues comprising an arginine-histidine-arginine triad and the aforementioned nucleophilic tyrosine residue (Swalla et al. 2003). The catalytic domain serves a similar mechanistic role, but can be structurally different, between different tyrosine recombinase systems.

Prominent members of the tyrosine recombinase family include integrases from coliphage I and prophage lambda, both of which help catalyze integration or excision of DNA elements from a phage genome onto a bacterial host. These integrases, as well as other tyrosine recombinases and serine recombinases, are capable of recognizing specific attachment sites on the phage genome, attP, and its counterpart on the bacterial genome, attB. Integration of phage DNA via site-specific recombination results in the generation of a linearized sequence flanked by newly modified attachment sites, called attL (left) and attR (right), respectively. Integrases of the tyrosine recombinase family require an accessory protein, known as the integration host factor (IHF), which binds and bends the DNA for integration. Problematically, the IHF is hard to introduce into the human system and requires a large attP site (about 200 bp) to initiate its mechanistic role (Merrick et al. 2018).

The tyrosine recombinase family also includes members, such as Cre, Flp, and Dre, which catalyze non-directional site-specific recombination in the absence of accessory proteins. These tyrosine recombinase systems have a number of advantages over their integrase counterparts, including small attachment sites (about 35 bp) and high efficiency of recombination in mammalian models (Kim et al. 2003; Lambert et al. 2007). Regardless of these inherent advantages, there are major drawbacks that limit their use. Due to the identical nature of the attachment sites, recombination mediated by tyrosine recombinases, such as Cre, often results in non-modification of these sites. This can lead to the occurrence of continual recombination events, even after the initial desired recombination effect, which may result in further excision and return to the undesired original DNA product. In some embodiments, the reversible nature of these tyrosine recombinase systems can be overcome by introduction of specialized mutated sites, whereupon recombination results in newly modified sites that do not undergo further recombination (Zhang et al. 2002). In some embodiments, their efficacy is still relatively low compared to that of the serine recombinase family.

### Serine Recombinase Family

As described herein, the serine recombinase family presents an attractive option for integrating large DNA payloads in a unidirectional manner that was not previously achievable with alternative gene transfer methods. It also does so without the burden of requiring accessory proteins or the presence of undesirable reverse reactions that affect its tyrosine recombinase family counterparts.

The serine recombinase family comprises resolvase/invertases, large serine recombinases (e.g., those included in Table 1), small serine recombinases, and transposases. Similar in function to the members of the tyrosine recombinase family, members of the serine recombinase family help mediate site-specific recombination events, but do so without accessory proteins and in one direction. Despite both tyrosine and serine recombinases controlling a number of recombination events, they are unrelated in protein sequence and structure, and work via different mechanisms.

Unlike tyrosine recombinases, serine recombinases rely predominantly on serine as their nucleophilic residue. DNA is cleaved by nucleophilic displacement of a DNA hydroxyl by the nucleophilic residue. In tyrosine recombinases, the result is creation of a 3′-phosphotyrosyl bridge, which contrasts with the formation of a 5′-phosphoserine linkage by serine recombinases (Grindley et al. 2006). Thus, serine recombinases do not form four-way intermediates or Holliday junctions, instead initiating double-stranded breaks at both sites without having to cleave one strand of each duplex at a time (Grindley et al. 2006). The double-stranded breaks are symmetrically located at the center of a crossover and are about 2 bp apart. Recombination events mediated by serine recombinases proceed by a unique subunit rotation mechanism that interchanges the positions of the cut DNA ends (Olorunniji et al. 2016).

Large serine recombinases (LSRs) comprise three primary structural domains: an amino-terminal catalytic domain, a recombinase domain, and a DNA-binding zinc ribbon domain (Van Duyne et al. 2013). The catalytic domain of LSRs contains a highly conserved nucleophilic serine residue surrounded by three arginine residues (Keenholtz et al. 2011). It serves as the prime site for formation of a synaptic complex between the recombinase and DNA, catalyzing the cleavage of DNA strands, and sequential subunit rotation during strand exchange (Bai et al. 2011; Van Duyne et al. 2013). The recombinase domain and neighboring zinc ribbon domain are both components of LSRs that further differentiate them from their small serine recombinase (SSRs) counterparts. Both domains play an integral role in binding DNA around the attP and attB attachment sites (Van Duyne et al. 2013). As exemplified by a serine recombinase from the Mycobacteriophage BxB1, these domains of LSRs are highly efficient and specific for their relatively small (about 40-50 bp) attachment sites attB and attP (Kim et al. 2003). In some embodiments, an HMMR computer software package (Eddy 2009) is used to identify the three domains typically associated with large serine recombinases: a resolvase/invertase domain (PF00239), a zinc ribbon domain (PF13408), and a recombinase domain Pfam (PF07508). Exemplary amino-terminal catalytic domains (PF00239) include amino acids 4-164 of SEQ ID NO: 58926, amino acids 5-154 of SEQ ID NO: 10611, amino acids 4-163 of SEQ ID NO: 33021, amino acids 4-162 of SEQ ID NO: 40191, amino acids 7-155 of SEQ ID NO: 5681, amino acids 4-155 of SEQ ID NO: 36231, amino acids 7-130 of SEQ ID NO: 34841, amino acids 13-160 of SEQ ID NO: 9906, amino acids 4-147 of SEQ ID NO: 21701, and amino acids 7-155 of SEQ ID NO: 7466. Exemplary recombinase domains (PF07508) include amino acids 190-276 of SEQ ID NO: 58926, amino acids 194-302 of SEQ ID NO: 10611, amino acids 191-287 of SEQ ID NO: 33021, amino acids 187-282 of SEQ ID NO: 40191, amino acids 179-261 of SEQ ID NO: 5681, amino acids 181-291 of SEQ ID NO: 36231, amino acids 191-262 of SEQ ID NO: 34841, amino acids 184-311 of SEQ ID NO: 9906, amino acids 170-259 of SEQ ID NO: 21701, and amino acids 184-261 of SEQ ID NO: 7466. Exemplary zinc ribbon domains (PF13408) include amino acids 296-350 of SEQ ID NO: 58926, amino acids 319-367 of SEQ ID NO: 10611, amino acids 304-357 of SEQ ID NO: 33021, amino acids 298-350 of SEQ ID NO: 40191, amino acids 281-352 of SEQ ID NO: 5681, amino acids 304-356 of SEQ ID NO: 36231, amino acids 279-335 of SEQ ID NO: 34841, amino acids 322-382 of SEQ ID NO: 9906, amino acids 273-332 of SEQ ID NO: 21701, and amino acids 281-352 of SEQ ID NO: 7466.

While there are mechanistic similarities among the LSRs, there are large differences in sequence identity between the LSRs, and the exact modalities responsible for targeting attachment sites for these recombinases are largely unknown (Van Duyne et al. 2013). Additionally, few large serine recombinases have been identified, and even fewer of those are capable of acting upon the human genome. Thus, the identification, characterization, and application of new LSRs would be useful in expanding the options for use in genetic engineering of non-bacterial cells (e.g., human cells) and for the manipulation of synthetic genetic circuits.

Described herein is a set of novel LSRs from a variety of phage (Table 1), identification of their respective attachment sites (attB and attP), and prediction of exemplary prospective attachment sites within the human genome. In general, an attachment site in the human genome (i.e., a human attachment site, “attH site”) can be identical or have homology to either an attB or an attP sequence of the present disclosure. It can also be identical or have homology to variants of an attB or attP sequence of the present disclosure (e.g., variants that include different central dinucleotides). An attH site identical or with homology to an attB site may recombine with an attP site (e.g., the attP site that normally recombines with the attB site). An attH site identical or with homology to an attP site may recombine with an attB site (e.g., the attB site that normally recombines with the attP site). For a given LSR and a given donor sequence for recombination (i.e., attD), there might be more than one putative attH site (e.g., sequences sharing high similarity with either an attB or attP) in a human genome. Methods for identification and characterization of these novel LSRs and human attachment sites are further discussed herein.

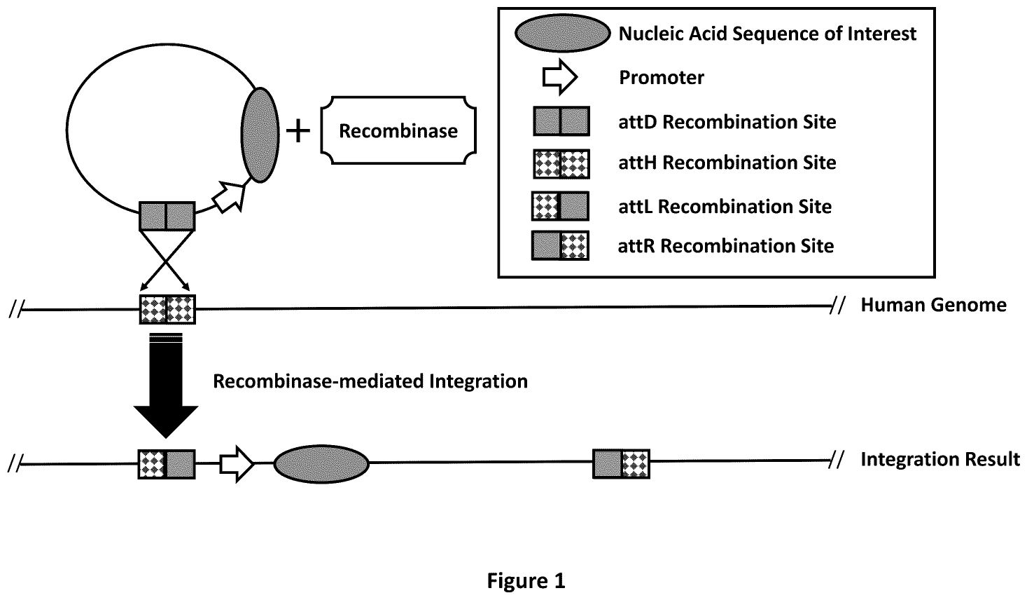

A “pair of attachment site sequences”, a “pair of an attB site sequence and an attP site sequence”, a “pair of an attH (or attA) site sequence and an attD site sequence”, and like terms, refer to pairs of attachment site sequences that share the same central dinucleotide where recombination can occur in the presence of the recombinase. In some embodiments, the central dinucleotide is non-palindromic. In some embodiments, the central dinucleotide is palindromic. In some embodiments, the central dinucleotide is selected from the group consisting of: AA, TT, GG, CC, AG, GA, AC, CA, TG, GT, TC, CT, AT, TA, CG, and GC. In some embodiments, a pair of a human attachment site (attH) sequence and a donor attachment site (attD) sequence comprise a central dinucleotide that differs from a homologous pair of attB and attP site sequences. In some embodiments, a pair of attachment site sequences are used in a recombination event, wherein one attachment site sequence is used in a host (e.g., human) genome (e.g., attH or attA) and the other attachment site sequence (e.g., attD) is part of an integrative vector (e.g., a DNA expression vector or plasmid). This is illustrated in FIG. 1 for an exemplary embodiment.

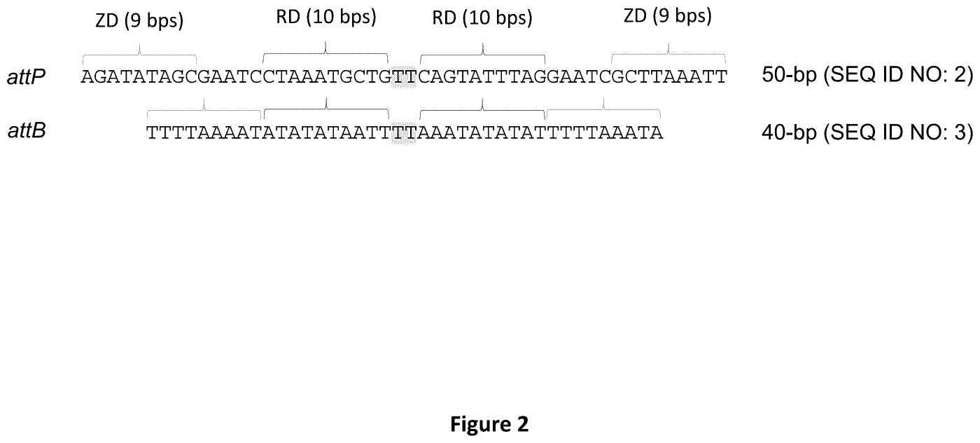

As shown in FIG. 2, in some embodiments, a pair of attachment site sequences comprise pairs of binding regions flanking the central dinucleotide. In some embodiments, a pair of attachment site sequences comprise a pair of recombinase domain (RD) binding regions directly 5′ and 3′ of the central dinucleotide. In some embodiments, the RD binding regions are each 10 base pairs long. In some embodiments, a pair of attachment site sequences comprise a pair of zinc ribbon domain (ZD) binding regions 5′ and 3′ of the RD binding regions. In some embodiments, the ZD binding regions are each 9 base pairs long. In some embodiments, an attachment site sequence comprises linkers between the RD binding regions and the ZD binding regions flanking the central dinucleotide. In some embodiments, a linker comprises 1, 2, 3, 4, 5, or more than 5 nucleotides. In some embodiments, an attachment site sequence comprises, from 5′ to 3′: a first ZD binding region, a first linker, a first RD binding region, a central dinucleotide, a second RD binding region, a second linker, and a second ZD binding region (e.g., see the attP site sequences shown in Table 1, Table 2 or Table 3 and any corresponding attD or attH sequences). In some embodiments, an attachment site sequence comprises, from 5′ to 3′: a first ZD binding region, a first RD binding region, a central dinucleotide, a second RD binding region, and a second ZD binding region (e.g., see the attB site sequences shown in Table 1, Table 2 or Table 3 and any corresponding attD or attH sequences).

In some embodiments, the present disclosure encompasses the use of attD sites (and corresponding attH (or attA) sites) that are variants of the attP or attB sites shown in Table 1, Table 2 or Table 3, where (i) the central dinucleotide is replaced with a different dinucleotide, e.g., where a central “CT” is replaced with “AG”, etc. and/or (ii) one or both of the linkers in an attP site are shortened from 5 to 4, 3, 2, 1 or 0 nucleotides, e.g., where “CCTAG” is replaced with “CCTA”, “CCT”, “CC”, “C” or absent.

In some embodiments, the present disclosure encompasses the use of attD sites (and corresponding attH (or attA) sites) that are variants of the attP or attB sites shown in Table 1, Table 2 or Table 3, where (i) the RD binding regions are shorter than 10 base pairs long, e.g., where 1, 2, or 3 nucleotides are removed from one or both ends of an RD binding region and/or (ii) the ZD binding regions are shorter than 9 base pairs long, e.g., where 1, 2, or 3 nucleotides are removed from one or both ends of a ZD binding region.

In some embodiments, in a pair of attachment site sequences used in a recombination event, wherein one attachment site sequence is present in a host (e.g., human) genome (e.g., attH or attA) and the other attachment site sequence (e.g., attD) is part of an integrative vector (e.g., a DNA expression vector or plasmid), the attachment site sequences share at least 50% identity (e.g., at least 50%, 55%, 60%, 65%, 70%, 75%, 80%, 85%, 90%, 91%, 92%, 93%, 94%, 95%, 96%, 97%, 98%, 99% or 100% identity) across the 30 to 50 base pairs (e.g., 30, 35, 40, 45, or 50 base pairs) surrounding the central dinucleotide sequences of the attachment sites. In some embodiments, in a pair of attachment site sequences, the sequences upstream and downstream of the central dinucleotide share 100% homology. In some embodiments, in a pair of attachment site sequences, the sequences upstream (e.g., 15 to 25 base pairs upstream, e.g., 15, 20, or 25 base pairs upstream) of the central dinucleotide share at least 50% homology (e.g., 50%, 55%, 60%, 65%, 70%, 75%, 80%, 85%, 90%, 95%, 99%, or 100% homology). In some embodiments, in a pair of attachment site sequences, the sequences downstream (e.g., 15 to 25 base pairs downstream, e.g., 15, 20, or 25 base pairs downstream) of the central dinucleotide share at least 50% homology (e.g., 50%, 55%, 60%, 65%, 70%, 75%, 80%, 85%, 90%, 91%, 92%, 93%, 94%, 95%, 96%, 97%, 98%, 99%, or 100% homology). In some embodiments, in a pair of attachment site sequences (e.g., attH and attD), the sequences upstream and/or downstream of the central dinucleotide in one attachment site (e.g., attH) share a certain percent identity with the sequences upstream and/or downstream of the central dinucleotide of the other attachment site (e.g., attD), for example, the upstream and/or downstream sequences are 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical in sequence. In some embodiments, in a pair of attachment site sequences (e.g., attH and attD), the sequence upstream of the central dinucleotide in one attachment site (e.g., attH) and the sequence upstream of the central dinucleotide in the other attachment site (e.g., attD) share at least 50%, e.g., 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identity. In some embodiments, in a pair of attachment site sequences (e.g., attH and attD), the sequence downstream of the central dinucleotide in one attachment site (e.g., attH) and the sequence downstream of the central dinucleotide in the other attachment site (e.g., attD) share at least 50%, e.g., 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identity.

In some embodiments, an LSR of the present disclosure comprises one or more protein domains selected from Table 1. In some embodiments, an LSR of the present disclosure comprises one, two, or three of the protein domains selected from Table 1. In some embodiments, an LSR of the present disclosure comprises an amino acid sequence at least 80% identical to a sequence selected from Table 1, Table 2 or Table 3. In some embodiments, an LSR of the present disclosure comprises an amino acid sequence at least 85% identical to a sequence selected from Table 1, Table 2 or Table 3. In some embodiments, an LSR of the present disclosure comprises an amino acid sequence at least 90% identical to a sequence selected from Table 1, Table 2 or Table 3. In some embodiments, an LSR of the present disclosure comprises an amino acid sequence at least 95% identical to a sequence selected from Table 1, Table 2 or Table 3. In some embodiments, an LSR of the present disclosure comprises an amino acid sequence at least 96% identical to a sequence selected from Table 1, Table 2 or Table 3. In some embodiments, an LSR of the present disclosure comprises an amino acid sequence at least 97% identical to a sequence selected from Table 1, Table 2 or Table 3. In some embodiments, an LSR of the present disclosure comprises an amino acid sequence at least 98% identical to a sequence selected from Table 1, Table 2 or Table 3. In some embodiments, an LSR of the present disclosure comprises an amino acid sequence at least 99% (e.g., 99.0%, 99.1%, 99.2%, 99.3%, 99.4%, 99.5%, 99.6%, 99.7%, 99.8%, or 99.9%) identical to a sequence selected from Table 1, Table 2 or Table 3. In some embodiments, an LSR of the present disclosure comprises an amino acid sequence that differs from a sequence selected from Table 1, Table 2 or Table 3by 1, 2, 3, 4, 5, 6, 7, 8, 9, 10, 15, or 20 amino acids where each difference may be in the form of a substitution, a deletion or an insertion. In some embodiments, an LSR of the present disclosure comprises an amino acid sequence identical to a sequence selected from Table 1, Table 2 or Table 3.

In some embodiments, an LSR of the present disclosure comprises an amino acid sequence at least 80%, 85%, 90%, 95%, 96%, 97%, 98%, 99%, 99.1%, 99.2%, 99.3%, 99.4%, 99.5%, 99.6%, 99.7%, 99.8%, 99.9%, or 100% identical to an amino acid sequence selected from SEQ ID NO: 58926, SEQ ID NO: 10611, SEQ ID NO: 33021, SEQ ID NO: 40191, SEQ ID NO: 5681, SEQ ID NO: 36231, SEQ ID NO: 34841, SEQ ID NO: 9906, SEQ ID NO: 21701, SEQ ID NO: 7466, SEQ ID NO: 57456, SEQ ID NO: 41066, SEQ ID NO: 41186, SEQ ID NO: 21126, SEQ ID NO: 1191, SEQ ID NO: 35081, SEQ ID NO: 18926, SEQ ID NO: 51806, SEQ ID NO: 58376, SEQ ID NO: 29771, SEQ ID NO: 21276, or SEQ ID NO: 36986. In some embodiments, an LSR of the present disclosure comprises an amino acid sequence that differs from a sequence selected from SEQ ID NO: 58926, SEQ ID NO: 10611, SEQ ID NO: 33021, SEQ ID NO: 40191, SEQ ID NO: 5681, SEQ ID NO: 36231, SEQ ID NO: 34841, SEQ ID NO: 9906, SEQ ID NO: 21701, SEQ ID NO: 7466, SEQ ID NO: 57456, SEQ ID NO: 41066, SEQ ID NO: 41186, SEQ ID NO: 21126, SEQ ID NO: 1191, SEQ ID NO: 35081, SEQ ID NO: 18926, SEQ ID NO: 51806, SEQ ID NO: 58376, SEQ ID NO: 29771, SEQ ID NO: 21276, or SEQ ID NO: 36986 by 1, 2, 3, 4, 5, 6, 7, 8, 9, 10, 15, or 20 amino acids where each difference may be in the form of a substitution, a deletion or an insertion.

In some embodiments, an LSR of the present disclosure recognizes cognate attachment sites. In some embodiments, an LSR of the present disclosure and its cognate attachment sites all have the same system ID in Table 1, Table 2 or Table 3 (i.e., they are all selected from or derived from sequences that are in the same row of Table 1, Table 2 or Table 3). In some embodiments, an attachment site is an attP site. In some embodiments, an attachment site is an attB site. In some embodiments, an attachment site is an attD (donor attachment) site. In some embodiments, an attachment site is an attH site. In some embodiments, an attachment site is an attA site. In some embodiments, an LSR of the present disclosure and its cognate attachment sites attB and attP all have the same system ID in Table 1, Table 2 or Table 3. In some embodiments, an LSR of the present disclosure and its cognate attachment sites attD and attH all have the same system ID in Table 1, Table 2 or Table 3. In some embodiments, an LSR of the present disclosure and its cognate attachment sites attD and attA all have the same system ID in Table 1, Table 2 or Table 3.

In some embodiments, an attP of the present disclosure comprises a nucleic acid sequence at least 80% identical to an attP sequence selected from Table 1, Table 2 or Table 3. In some embodiments, an attP of the present disclosure comprises a nucleic acid sequence at least 85% identical to an attP sequence selected from Table 1, Table 2 or Table 3. In some embodiments, an attP of the present disclosure comprises a nucleic acid sequence at least 90% identical to an attP sequence selected from Table 1, Table 2 or Table 3. In some embodiments, an attP of the present disclosure comprises a nucleic acid sequence at least 95% identical to an attP sequence selected from Table 1, Table 2 or Table 3. In some embodiments, an attP of the present disclosure comprises a nucleic acid sequence at least 96% identical to an attP sequence selected from Table 1, Table 2 or Table 3. In some embodiments, an attP of the present disclosure comprises a nucleic acid sequence at least 97% identical to an attP sequence selected from Table 1, Table 2 or Table 3. In some embodiments, an attP of the present disclosure comprises a nucleic acid sequence at least 98% identical to an attP sequence selected from Table 1, Table 2 or Table 3. In some embodiments, an attP of the present disclosure comprises a nucleic acid sequence at least 99% identical to an attP sequence selected from Table 1, Table 2 or Table 3. In some embodiments, an attP of the present disclosure comprises a nucleic acid sequence identical to an attP sequence selected from Table 1, Table 2 or Table 3.

In some embodiments, an attB of the present disclosure comprises a nucleic acid sequence at least 80% identical to an attB sequence selected from Table 1, Table 2 or Table 3. In some embodiments, an attB of the present disclosure comprises a nucleic acid sequence at least 85% identical to an attB sequence selected from Table 1, Table 2 or Table 3. In some embodiments, an attB of the present disclosure comprises a nucleic acid sequence at least 90% identical to an attB sequence selected from Table 1, Table 2 or Table 3. In some embodiments, an attB of the present disclosure comprises a nucleic acid sequence at least 95% identical to an attB sequence selected from Table 1, Table 2 or Table 3. In some embodiments, an attB of the present disclosure comprises a nucleic acid sequence at least 96% identical to an attB sequence selected from Table 1, Table 2 or Table 3. In some embodiments, an attB of the present disclosure comprises a nucleic acid sequence at least 97% identical to an attB sequence selected from Table 1, Table 2 or Table 3. In some embodiments, an attB of the present disclosure comprises a nucleic acid sequence at least 98% identical to an attB sequence selected from Table 1, Table 2 or Table 3. In some embodiments, an attB of the present disclosure comprises a nucleic acid sequence at least 99% identical to an attB sequence selected from Table 1, Table 2 or Table 3. In some embodiments, an attB of the present disclosure comprises a nucleic acid sequence identical to an attB sequence selected from Table 1, Table 2 or Table 3.

In some embodiments, an attD of the present disclosure comprises a nucleic acid sequence at least 80% identical to an attD sequence selected from Table 1, Table 2 or Table 3. In some embodiments, an attD of the present disclosure comprises a nucleic acid sequence at least 85% identical to an attD sequence selected from Table 1, Table 2 or Table 3. In some embodiments, an attD of the present disclosure comprises a nucleic acid sequence at least 90% identical to an attD sequence selected from Table 1, Table 2 or Table 3. In some embodiments, an attD of the present disclosure comprises a nucleic acid sequence at least 95% identical to an attD sequence selected from Table 1, Table 2 or Table 3. In some embodiments, an attD of the present disclosure comprises a nucleic acid sequence at least 96% identical to an attD sequence selected from Table 1, Table 2 or Table 3. In some embodiments, an attD of the present disclosure comprises a nucleic acid sequence at least 97% identical to an attD sequence selected from Table 1, Table 2 or Table 3. In some embodiments, an attD of the present disclosure comprises a nucleic acid sequence at least 98% identical to an attD sequence selected from Table 1, Table 2 or Table 3. In some embodiments, an attD of the present disclosure comprises a nucleic acid sequence at least 99% identical to an attD sequence selected from Table 1, Table 2 or Table 3. In some embodiments, an attD of the present disclosure comprises a nucleic acid sequence identical to an attD sequence selected from Table 1, Table 2 or Table 3.

In some embodiments, an attH of the present disclosure comprises a nucleic acid sequence at least 80% identical to an attH sequence selected from Table 1, Table 2 or Table 3. In some embodiments, an attH of the present disclosure comprises a nucleic acid sequence at least 85% identical to an attH sequence selected from Table 1, Table 2 or Table 3. In some embodiments, an attH of the present disclosure comprises a nucleic acid sequence at least 90% identical to an attH sequence selected from Table 1, Table 2 or Table 3. In some embodiments, an attH of the present disclosure comprises a nucleic acid sequence at least 95% identical to a sequence selected from Table 1, Table 2 or Table 3. In some embodiments, an attH of the present disclosure comprises a nucleic acid sequence at least 96% identical to an attH sequence selected from Table 1, Table 2 or Table 3. In some embodiments, an attH of the present disclosure comprises a nucleic acid sequence at least 97% identical to an attH sequence selected from Table 1, Table 2 or Table 3. In some embodiments, an attH of the present disclosure comprises a nucleic acid sequence at least 98% identical to an attH sequence selected from Table 1, Table 2 or Table 3. In some embodiments, an attH of the present disclosure comprises a nucleic acid sequence at least 99% identical to an attH sequence selected from Table 1, Table 2 or Table 3. In some embodiments, an attH of the present disclosure comprises a nucleic acid sequence identical to an attH sequence selected from Table 1, Table 2 or Table 3.

In some embodiments, a pair of attachment site sequences have the same system ID in Table 1, Table 2 or Table 3. In some embodiments, a pair of attachment site sequences attB and attP have the same system ID in Table 1, Table 2 or Table 3. In some embodiments, a pair of attachment site sequences attB and attP each comprise a nucleic acid sequence at least 80%, 85%, 90%, 95%, 96%, 97%, 98%, or 99% identical to a sequence selected from Table 1, Table 2 or Table 3. In some embodiments, a pair of attachment site sequences attB and attP each comprise a nucleic acid sequence at least 80%, 85%, 90%, 95%, 96%, 97%, 98%, or 99% identical to a sequence selected from Table 1, Table 2 or Table 3 and have the same system ID in Table 1, Table 2 or Table 3. In some embodiments, a pair of attachment site sequences attD and attH have the same system ID in Table 1, Table 2 or Table 3. In some embodiments, a pair of attachment site sequences attD and attH each comprise a nucleic acid sequence at least 80%, 85%, 90%, 95%, 96%, 97%, 98%, or 99% identical to a sequence selected from Table 1, Table 2 or Table 3. In some embodiments, a pair of attachment site sequences attD and attH each comprise a nucleic acid sequence at least 80%, 85%, 90%, 95%, 96%, 97%, 98%, or 99% identical to a sequence selected from Table 1, Table 2 or Table 3 and have the same system ID in Table 1, Table 2 or Table 3.

In some embodiments, an attachment site sequence present in a host (e.g., human) genome (e.g., attH or attA) shares an identical central dinucleotide sequence with an attP, attB, or attH in Table 1, Table 2 or Table 3. In some embodiments, an attachment site sequence present in a host (e.g., human) genome (e.g., attH or attA) contains no mismatches relative to the central dinucleotide sequence of an attP, attB, or attH in Table 1, Table 2 or Table 3. In some embodiments, an attachment site sequence present in a host (e.g., human) genome (e.g., attH or attA) shares at least 50% identity (e.g., at least 50%, 55%, 60%, 65%, 70%, 75%, 80%, 85%, 90%, 95%, 99% or 100% identity) with the 30 to 50 base pairs (e.g., 30, 35, 40, 45, or 50 base pairs) surrounding the central dinucleotide of an attP, attB, or attH in Table 1, Table 2 or Table 3. In some embodiments, the 15 to 25 nucleotides located immediately 5′ or upstream of the central dinucleotide of an attachment site sequence present in a host (e.g., human) genome (e.g., attH or attA) share at least 50% sequence identity (e.g., at least 50%, 55%, 60%, 65%, 70%, 75%, 80%, 85%, 90%, 95%, 99% or 100% identity) with the 15 to 25 nucleotides located immediately 5′ or upstream of the central dinucleotide of an attP, attB, or attH in Table 1, Table 2 or Table 3. In some embodiments, the 15 to 25 nucleotides located immediately 3′ or downstream of the central dinucleotide of an attachment site sequence present in a host (e.g., human) genome (e.g., attH or attA) share at least 50% sequence identity (e.g., at least 50%, 55%, 60%, 65%, 70%, 75%, 80%, 85%, 90%, 95%, 99% or 100% identity) with the 15 to 25 nucleotides located immediately 3′ or downstream of the central dinucleotide of an attP, attB, or attH in Table 1, Table 2 or Table 3.

In some embodiments, an attachment site sequence present in a host (e.g., human) genome (e.g., attH or attA) can contain up to 15 nucleotide mismatches (e.g., 1, 2, 3, 4, 5, 6, 7, 8, 9, 10, 11, 12, 13, 14, or 15 mismatches) across the 30 base pairs surrounding the central dinucleotide of an attP, attB, or attH in Table 1, Table 2 or Table 3. In some embodiments, an attachment site sequence present in a host (e.g., human) genome (e.g., attH or attA) can contain up to 20 nucleotide mismatches (e.g., 1, 2, 3, 4, 5, 6, 7, 8, 9, 10, 11, 12, 13, 14, 15, 16, 17, 18, 19, or 20 mismatches) across the 40 base pairs surrounding the central dinucleotide of an attP, attB, or attH in Table 1, Table 2 or Table 3. In some embodiments, an attachment site sequence present in a host (e.g., human) genome (e.g., attH or attA) can contain up to 25 nucleotide mismatches (e.g., 1, 2, 3, 4, 5, 6, 7, 8, 9, 10, 11, 12, 13, 14, 15, 16, 17, 18, 19, 20, 21, 22, 23, 24, or 25 mismatches) across the 50 base pairs surrounding the central dinucleotide of an attP or attH in Table 1, Table 2 or Table 3.

In some embodiments, the 15 nucleotides located immediately 5′ or upstream of the central dinucleotide of an attachment site sequence present in a host (e.g., human) genome (e.g., attH or attA) can contain up to 7 nucleotide mismatches (e.g., 1, 2, 3, 4, 5, 6, or 7 mismatches) relative to the 15 nucleotides located immediately 5′ or upstream of the central dinucleotide of an attP, attB, or attH in Table 1, Table 2 or Table 3. In some embodiments, the 20 nucleotides located immediately 5′ or upstream of the central dinucleotide of an attachment site sequence present in a host (e.g., human) genome (e.g., attH or attA) can contain up to 10 nucleotide mismatches (e.g., 1, 2, 3, 4, 5, 6, 7, 8, 9, or 10 mismatches) relative to the 20 nucleotides located immediately 5′ or upstream of the central dinucleotide of an attP, attB, or attH in Table 1, Table 2 or Table 3. In some embodiments, the 25 nucleotides located immediately 5′ or upstream of the central dinucleotide of an attachment site sequence present in a host (e.g., human) genome (e.g., attH or attA) can contain up to 13 nucleotide mismatches (e.g., 1, 2, 3, 4, 5, 6, 7, 8, 9, 10, 11, 12, or 13 mismatches) relative to the 25 nucleotides located immediately 5′ or upstream of the central dinucleotide of an attP or attH in Table 1, Table 2 or Table 3.

In some embodiments, the 15 nucleotides located immediately 3′ or downstream of the central dinucleotide of an attachment site sequence present in a host (e.g., human) genome (e.g., attH or attA) can contain up to 7 nucleotide mismatches (e.g., 1, 2, 3, 4, 5, 6, or 7 mismatches) relative to the 15 nucleotides located immediately 3′ or downstream of the central dinucleotide of an attP, attB, or attH in Table 1, Table 2 or Table 3. In some embodiments, the 20 nucleotides located immediately 3′ or downstream of the central dinucleotide of an attachment site sequence present in a host (e.g., human) genome (e.g., attH or attA) can contain up to 10 nucleotide mismatches (e.g., 1, 2, 3, 4, 5, 6, 7, 8, 9, or 10 mismatches) relative to the 20 nucleotides located immediately 3′ or downstream of the central dinucleotide of an attP, attB, or attH in Table 1, Table 2 or Table 3. In some embodiments, the 25 nucleotides located immediately 3′ or downstream of the central dinucleotide of an attachment site sequence present in a host (e.g., human) genome (e.g., attH or attA) can contain up to 13 nucleotide mismatches (e.g., 1, 2, 3, 4, 5, 6, 7, 8, 9, 10, 11, 12, or 13 mismatches) relative to the 25 nucleotides located immediately 3′ or downstream of the central dinucleotide of an attP or attH in Table 1, Table 2 or Table 3.

In some embodiments, an attachment site sequence (e.g., attD) present on an exogenous nucleic acid, e.g., exogenous DNA (e.g., an expression vector, such as a DNA plasmid) shares an identical central dinucleotide sequence as an attD, attP or attB in Table 1, Table 2 or Table 3. In some embodiments, an attachment site sequence (e.g., attD) present on an exogenous nucleic acid, e.g., exogenous DNA (e.g., an expression vector, such as a DNA plasmid) contains no mismatches relative to the central dinucleotide sequence of an attD, attP, or attB in Table 1, Table 2 or Table 3. In some embodiments, an attachment site sequence (e.g., attD) present on an exogenous nucleic acid, e.g., exogenous DNA (e.g., an expression vector, such as a DNA plasmid) shares at least 50% identity (e.g., at least 50%, 55%, 60%, 65%, 70%, 75%, 80%, 85%, 90%, 95%, 99% or 100% identity) with the 30 to 50 base pairs (e.g., 30, 35, 40, 45, or 50 base pairs) surrounding the central dinucleotide of an attD, attP, or attB in Table 1, Table 2 or Table 3. In some embodiments, the 15 to 25 nucleotides located immediately 5′ or upstream of the central dinucleotide of an attachment site sequence (e.g., attD) present on an exogenous nucleic acid, e.g., exogenous DNA (e.g., an expression vector, such as a DNA plasmid) share at least 50% sequence identity (e.g., at least 50%, 55%, 60%, 65%, 70%, 75%, 80%, 85%, 90%, 95%, 99% or 100% identity) with the 15 to 25 nucleotides located immediately 5′ or upstream of the central dinucleotide of an attD, attP, or attB in Table 1, Table 2 or Table 3. In some embodiments, the 15 to 25 nucleotides located immediately 3′ or downstream of the central dinucleotide of an attachment site sequence (e.g., attD) present on an exogenous nucleic acid, e.g., exogenous DNA (e.g., an expression vector, such as a DNA plasmid) share at least 50% sequence identity (e.g., at least 50%, 55%, 60%, 65%, 70%, 75%, 80%, 85%, 90%, 95%, 99% or 100% identity) with the 15 to 25 nucleotides located immediately 3′ or downstream of the central dinucleotide of an attD, attP, or attB in Table 1, Table 2 or Table 3.

In some embodiments, an attachment site sequence (e.g., attD) present on an exogenous nucleic acid, e.g., exogenous DNA (e.g., an expression vector, such as a DNA plasmid) can contain up to 15 nucleotide mismatches (e.g., 1, 2, 3, 4, 5, 6, 7, 8, 9, 10, 11, 12, 13, 14, or 15 mismatches) across the 30 base pairs surrounding the central dinucleotide of an attD, attP, or attB in Table 1, Table 2 or Table 3. In some embodiments, an attachment site sequence (e.g., attD) present on an exogenous nucleic acid, e.g., exogenous DNA (e.g., an expression vector, such as a DNA plasmid) can contain up to 20 nucleotide mismatches (e.g., 1, 2, 3, 4, 5, 6, 7, 8, 9, 10, 11, 12, 13, 14, 15, 16, 17, 18, 19, or 20 mismatches) across the 40 base pairs surrounding the central dinucleotide of an attD, attP, or attB in Table 1, Table 2 or Table 3. In some embodiments, an attachment site sequence (e.g., attD) present on an exogenous nucleic acid, e.g., exogenous DNA (e.g., an expression vector, such as a DNA plasmid) can contain up to 25 nucleotide mismatches (e.g., 1, 2, 3, 4, 5, 6, 7, 8, 9, 10, 11, 12, 13, 14, 15, 16, 17, 18, 19, 20, 21, 22, 23, 24, or 25 mismatches) across the 50 base pairs surrounding the central dinucleotide of an attD or attP in Table 1, Table 2 or Table 3.

In some embodiments, the 15 nucleotides located immediately 5′ or upstream of the central dinucleotide of an attachment site sequence (e.g., attD) present on an exogenous nucleic acid, e.g., exogenous DNA (e.g., an expression vector, such as a DNA plasmid) can contain up to 7 nucleotide mismatches (e.g., 1, 2, 3, 4, 5, 6, or 7 mismatches) relative to the 15 nucleotides located immediately 5′ or upstream of the central dinucleotide of an attD, attP, or attB in Table 1, Table 2 or Table 3. In some embodiments, the 20 nucleotides located immediately 5′ or upstream of the central dinucleotide of an attachment site sequence (e.g., attD) present on an exogenous nucleic acid, e.g., exogenous DNA (e.g., an expression vector, such as a DNA plasmid) can contain up to 10 nucleotide mismatches (e.g., 1, 2, 3, 4, 5, 6, 7, 8, 9, or 10 mismatches) relative to the 20 nucleotides located immediately 5′ or upstream of the central dinucleotide of an attD, attP, or attB in Table 1, Table 2 or Table 3. In some embodiments, the 25 nucleotides located immediately 5′ or upstream of the central dinucleotide of an attachment site sequence (e.g., attD) present on an exogenous nucleic acid, e.g., exogenous DNA (e.g., an expression vector, such as a DNA plasmid) can contain up to 13 nucleotide mismatches (e.g., 1, 2, 3, 4, 5, 6, 7, 8, 9, 10, 11, 12, or 13 mismatches) relative to the 25 nucleotides located immediately 5′ or upstream of the central dinucleotide of an attD or attP in Table 1, Table 2 or Table 3.

In some embodiments, the 15 nucleotides located immediately 3′ or downstream of the central dinucleotide of an attachment site sequence (e.g., attD) present on an exogenous nucleic acid, e.g., exogenous DNA (e.g., an expression vector, such as a DNA plasmid) can contain up to 7 nucleotide mismatches (e.g., 1, 2, 3, 4, 5, 6, or 7 mismatches) relative to the 15 nucleotides located immediately 3′ or downstream of the central dinucleotide of an attD, attP, or attB in Table 1, Table 2 or Table 3. In some embodiments, the 20 nucleotides located immediately 3′ or downstream of the central dinucleotide of an attachment site sequence (e.g., attD) present on an exogenous nucleic acid, e.g., exogenous DNA (e.g., an expression vector, such as a DNA plasmid) can contain up to 10 nucleotide mismatches (e.g., 1, 2, 3, 4, 5, 6, 7, 8, 9, or 10 mismatches) relative to the 20 nucleotides located immediately 3′ or downstream of the central dinucleotide of an attD, attP, or attB in Table 1, Table 2 or Table 3. In some embodiments, the 25 nucleotides located immediately 3′ or downstream of the central dinucleotide of an attachment site sequence (e.g., attD) present on an exogenous nucleic acid, e.g., exogenous DNA (e.g., an expression vector, such as a DNA plasmid) can contain up to 13 nucleotide mismatches (e.g., 1, 2, 3, 4, 5, 6, 7, 8, 9, 10, 11, 12, or 13 mismatches) relative to the 25 nucleotides located immediately 3′ or downstream of the central dinucleotide of an attD or attP in Table 1, Table 2 or Table 3.

### Application of Large Serine Recombinases

The LSRs of the present disclosure can be used to incorporate an exogenous nucleic acid, e.g., exogenous DNA into a human chromosome. The methods and compositions described herein enable the targeted insertion of large nucleic acid sequences (e.g., DNA sequences) into the human genome that was not possible using prior methods and compositions for genetic modification. In some embodiments, the set of LSRs and characterized human attachment sites allow for design of human gene expression systems (e.g., expression vectors). In some embodiments, a human gene expression system comprises a nucleic acid encoding an exogenous nucleic acid sequence of interest operably linked to a promoter that is operable in a human cell. In some embodiments, the nucleic acid encoding the nucleic acid sequence of interest further comprises a donor attachment site (attD). In some embodiments an attD site comprises an attP or attB site that is cognate with a large serine recombinase included in Table 1, Table 2 or Table 3. In some embodiments, an attD site comprises any of the aforementioned variant attP or attB sites of the present disclosure including a sequence that is at least 80% identical to an attP or attB site that is cognate with a large serine recombinase included in Table 1, Table 2 or Table 3. In some embodiments, a promoter of a gene expression system of the present disclosure is constitutive. In some embodiments, a promoter of a gene expression system of the present disclosure is inducible. In some embodiments, a gene expression system of the present disclosure may contain other regulatory elements, including enhancers. In some embodiments, a vector comprises a nucleic acid encoding a nucleic acid sequence of interest and a donor attachment site (attD). In some embodiments, the vector can be a DNA vector. In some embodiments, the DNA vector can be a plasmid, a nanoplasmid, a minicircle, or a doggybone DNA (dbDNA). In some embodiments, the DNA vector can be single-stranded. In some embodiments, the DNA vector can be double-stranded. In some embodiments, the DNA vector can be circular. In some embodiments, the DNA vector can be linear, e.g., linearized prior to delivery to a human cell. In some embodiments, an integration system of the present disclosure comprises an LSR, or a nucleic acid encoding an LSR, such as an mRNA or DNA sequence encoding an LSR. In some embodiments, the LSR is an LSR present in Table 1, Table 2 or Table 3. In some embodiments, an integration system comprises an LSR and a nucleic acid encoding a nucleic acid sequence of interest and an attD. In some embodiments, an integration system comprises one or more nucleic acids encoding a nucleic acid sequence of interest, an attD, and an LSR. In some embodiments, a gene expression system comprises a DNA (e.g., a plasmid DNA) encoding a nucleic acid sequence of interest and an attD, and an mRNA encoding an LSR. In some embodiments, an integration system of the present disclosure or a component thereof can be delivered into a human cell via a lipid nanoparticle (LNP). In some embodiments, an mRNA encoding an LSR comprises a modification. In some embodiments, the modification is or comprises: modified nucleotides as described herein (e.g., 1-methyl-pseudouridine and/or N1-methyl-pseudouridine), a 5′ modification (e.g., a 5′ cap), an untranslated region (UTR) (e.g., a 5′ and/or 3′ UTR), a 3′ modification (e.g., a polyA tail), or combinations thereof. Upon delivery into a human cell, an LSR of the present disclosure can mediate recombination between an attD of a nucleic acid encoding a nucleic acid sequence of interest with a human attachment site (attH), e.g., an attH of Table 1, Table 2 or Table 3, present in the genome of the cell. As a result, a relatively large exogenous nucleic acid sequence of interest could be integrated into a desired location of the human genome.

In some embodiments, LSRs of the present disclosure (e.g., in Table 1, Table 2 or Table 3) can be used to mediate excision or inversion events of the human genome. If both attachment sites exist on the same nucleic acid molecule and in the same direction, a recombinase of the present disclosure (e.g., in Table 1, Table 2 or Table 3) would be capable of mediating excision of any DNA between the attachment sites. Furthermore, if both attachment sites exist on the same nucleic acid molecule but in inverse orientations, the recombinase could be used to mediate inversion of any DNA in between the sites. A combination of these different recombination events mediated by LSRs of the present disclosure (e.g., in Table 1, Table 2 or Table 3) may be employed by one skilled in the art for precise genetic engineering of the human genome.

In some embodiments, the present disclosure provides insertion of a “landing pad” comprising an attachment site (e.g., an attH, attA, attB or attP sequence of the present disclosure) in the human genome. In some embodiments, LSRs of the present disclosure can be used to meditate integration at a landing pad comprising an attachment site. A landing pad can be inserted via any method known in the art, including, for example, prime editing. In some embodiments, insertion of a landing pad may use a prime editing gRNA (pegRNA) in conjunction with a prime editor (PE). The pegRNA is a gRNA with a primer binding sequence (PBS) and a donor template containing the desired RNA sequence added at one of the termini, e.g., the 3′ end. The PE:pegRNA complex binds to the target DNA, and the nickase domain of the prime editor nicks only one strand, generating a flap. The PBS, located on the pegRNA, binds to the DNA flap and the edited RNA sequence is reverse transcribed using the reverse transcriptase domain of the prime editor. The edited strand is incorporated into the DNA at the end of the nicked flap, and the target DNA is repaired with the new reverse transcribed DNA. The original DNA segment is removed by a cellular endonuclease. This leaves one strand edited (e.g., with an inserted landing pad), and one strand unedited. In other embodiments, a landing pad may be inserted via CRISPR-mediated homologous recombination with a donor template or using a base editor.

In some embodiments, a human cell is a quiescent cell. In some embodiments, a human cell is or comprises: an osteoblast, a chondrocyte, an adipocyte, a skeletal muscle cell, a cardiac muscle cell, a neuron, an astrocyte, an oligodendrocyte, a Schwann cell, a retinal cell (e.g., a retinal ganglion cell, a photoreceptor cell, or a retinal epithelium cell), a corneal cell, a skin cell, a monocyte, a macrophage, a neutrophil, a basophil, an eosinophil, an erythrocyte, a megakaryocyte, a dendritic cell, a T-lymphocyte, a B-lymphocyte, an NK-cell, a gastric cell, an intestinal cell, a smooth muscle cell, a vascular cell, a bladder cell, a pancreatic alpha cell, a pancreatic beta cell, a pancreatic delta cell, a liver cell (e.g., a hepatocyte, a hepatic stellate cell, a Kupffer cell, or a liver sinusoidal endothelial cell), a renal cell, an adrenal cell, or a lung cell. In certain embodiments, the human cell is a photoreceptor cell, a retinal epithelial cell or a retinal ganglion cell. In some embodiments, a human cell is a stem cell or progenitor cell. In some embodiments, a stem cell or progenitor cell is or comprises: a mesenchymal stem cell, a hematopoietic stem cell, a neuronal stem cell, a retinal stem cell, a cardiac muscle stem cell, a skeletal muscle stem cell, an adipose tissue derived stem cell, a chondrogenic stem cell, a liver stem cell, a kidney stem cell, a pancreatic stem cell, an embryonic stem cell, an induced pluripotent stem cell, or a fate-converted stem or progenitor cell. In some embodiments, a human cell is a hematopoietic stem cell or a hematopoietic progenitor cell.

### Nucleic Acid Sequence of Interest

The LSRs of the present disclosure can be used to integrate any nucleic acid sequence of interest into a cell, e.g., in the cell of a subject. In some embodiments, the nucleic acid sequence of interest may include a prokaryotic DNA sequence, cDNA from eukaryotic mRNA, a genomic DNA sequence from eukaryotic (e.g., mammalian) DNA, or a synthetic DNA sequence.

In some embodiments, the nucleic acid sequence of interest may encode a gene product. In some embodiments, a gene product comprises an antibody, an antigen, an enzyme, a growth factor, a receptor (e.g., cell surface, cytoplasmic, or nuclear), a hormone, a lymphokine, a cytokine, a chemokine, a reporter, a functional fragment of any of the above, or a combination of any of the above. In some embodiments, a gene product comprises a miRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

In some embodiments, the nucleic acid sequence of interest may encode a therapeutic protein or other gene product that confers a desired feature to the modified cell. In some embodiments, the therapeutic protein may be a protein deficient in the cell or subject. In some embodiments, for example, therapeutic proteins include, but are not limited to, those deficient in lysosomal storage disorders, such as alpha-L-iduronidase, arylsulfatase A, beta-glucocerebrosidase, acid sphingomyelinase, and alpha- and beta-galactosidase; and those deficient in hemophilia such as Factor VIII and Factor IX. Other examples of therapeutic proteins include, but are not limited to, antibodies or antibody fragments (e.g., scFv) such as those targeting pathogenic proteins (e.g., tau, alpha-synuclein, and beta-amyloid protein) and those targeting cancer cells (e.g., chimeric antigen receptors (CARs)).

In some embodiments, the nucleic acid sequence of interest may encode a protein involved in immune regulation, or an immunomodulatory protein. In some embodiments, for example, such proteins include, PD-L1, CTLA-4, M-CSF, IL-4, IL-6, IL-10, IL-11, IL-13, TGF-β1, and various isoforms thereof. By way of example, in some embodiments, the nucleic acid sequence of interest may encode an isoform of HLA-G (e.g., HLA-G1, -G2, -G3, -G4, -G5, -G6, or -G7) or HLA-E; allogeneic cells expressing such a nonclassical MHC class I molecule may be less immunogenic and better tolerated when transplanted into a human patient who is not the source of the cells, making “universal” cell therapy possible.

In some embodiments, the nucleic acid sequence of interest may encode a gene product that confers therapeutic value, e.g., a new therapeutic activity to the cell. In some embodiments, exemplary gene products are polypeptides such as a chimeric antigen receptor (CAR) or antigen-binding fragment thereof, a T cell receptor or antigen binding fragment thereof, a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof. It is to be understood that the present disclosure is not limited to any particular gene product and that the selection of a gene product will depend on the application.

In some embodiments, the nucleic acid sequence of interest may encode a cytokine. In some embodiments, expression of a cytokine from a modified cell generated using a method as described herein allows for localized dosing of the cytokine in vivo (e.g., within a subject in need thereof) and/or avoids a need to systemically administer a high-dose of the cytokine to a subject in need thereof (e.g., a lower dose of the cytokine may be administered). In some embodiments, the risk of dose-limiting toxicities associated with administering a cytokine is reduced while cytokine mediated cell functions are maintained. In some embodiments, to facilitate cell function without the need to additionally administer high-doses of soluble cytokines, a partial or full peptide of one or more of IL2, IL4, IL6, IL7, IL9, IL10, IL11, IL 12, IL15, IL18, IL21, IFN-α, IFN-β and/or their respective receptor is introduced to the cell to enable cytokine signaling with or without the expression of the cytokine itself, thereby maintaining or improving cell growth, proliferation, expansion, and/or effector function with reduced risk of cytokine toxicities. In some embodiments, the introduced cytokine and/or its respective native or modified receptor for cytokine signaling are expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, the nucleic acid sequence of interest may encode IL2, IL3, IL4, IL6, IL7, IL9, IL10, IL11, IL 12, IL13, IL15, IL21, GM-CSF, IFN-α, IFN-b, IFN-g, erythropoietin, and/or the respective cytokine receptor. In some embodiments, the nucleic acid sequence of interest may encode CCL3, TNFα, CCL23, IL2RB, IL12RB2, or IRF7.

In some embodiments, the nucleic acid sequence of interest may encode a chemokine and/or the respective chemokine receptor. In some embodiments, a chemokine receptor can be, but is not limited to, CCR2, CCR5, CCR8, CX3C1, CX3CR1, CXCR1, CXCR2, CXCR3A, CXCR3B, or CXCR2. In some embodiments, a chemokine can be, but is not limited to, CCL7, CCL19, or CXL14.

As used herein, the term “chimeric antigen receptor” or “CAR” refers to a receptor protein that has been modified to give cells expressing the CAR the new ability to target a specific protein. Within the context of the disclosure, a cell modified to comprise a CAR or an antigen binding fragment may be used for immunotherapy to target and destroy cells associated with a disease or disorder, e.g., cancer cells.

CARs of interest can include, but are not limited to, a CAR targeting mesothelin, EGFR, HER2 and/or MICA/B. To date, mesothelin-targeted CAR T-cell therapy has shown early evidence of efficacy in a phase I clinical trial of subjects having mesothelioma, non-small cell lung cancer, and breast cancer (NCT02414269). Similarly, CARs targeting EGFR, HER2 and MICA/B have shown promise in early studies (see, e.g., Li et al. (2018), Cell Death & Disease, 9(177); Han et al. (2018) Am. J. Cancer Res., 8(1):106-119; and Demoulin (2017) Future Oncology, 13(8); the entire contents of each of which are expressly incorporated herein by reference in their entireties).

In some embodiments, the nucleic acid sequence of interest may encode any suitable CAR, NK cell specific CAR (NK-CAR), T cell specific CAR, or other binder that targets a cell, e.g., an NK cell, to a target cell, e.g., a cell associated with a disease or disorder, may be expressed in the modified cells provided herein. Exemplary CARs, and binders, include, but are not limited to, bi-specific antigen binding CARs, switchable CARs, dimerizable CARs, split CARs, multi-chain CARs, inducible CARs, CARs and binders that bind BCMA, androgen receptor, PSMA, PSCA, Muc1, HPV viral peptides (i.e., E7), EBV viral peptides, WT1, CEA, EGFR, EGFRVIII, IL13Ra2, GD2, CA125, EpCAM, Muc16, carbonic anhydrase IX (CAIX), CCR1, CCR4, carcinoembryonic antigen (CEA), CD3, CD5, CD7, CD10, CD19, CD20, CD22, CD23, CD24, CD26, CD30, CD33, CD34, CD35, CD38 CD41, CD44, CD44V6, CD49f, CD56, CD70, CD92, CD99, CD123, CD133, CD135, CD148, CD150, CD261, CD362, CLEC12A, MDM2, CYPIB, livin, cyclin 1, NKp30, NKp46, DNAMI, NKp44, CA9, PD1, PDL1, an antigen of cytomegalovirus (CMV), epithelial glycoprotein-40 (EGP-40), GPRC5D, receptor tyrosine kinases erb-B2,3,4, EGFIR, ERBB folate binding protein (FBP), fetal acetylcholine receptor (AChR), folate receptor-a, ganglioside G3 (GD3) human Epidermal Growth Factor Receptor 2 (HER-2), human telomerase reverse transcriptase (hTERT), ICAM-1, Integrin B7, Interleukin-13 receptor subunit alpha-2 (IL-13Ra2), K-light chain, kinase insert domain receptor (KDR), Lewis A (CA19.9), Lewis Y (Le Y), L1 cell adhesion molecule (LI-CAM), LILRB2, melanoma antigen family A 1 (MAGE-A1), MICA/B, Mucin 16 (Muc-16), NKCSI, NKG2D ligands, c-Met, cancer-testis antigen NY-ESO-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), a pathogen antigen, or any suitable combination thereof.

In some embodiments, the nucleic acid sequence of interest may encode a protein or polypeptide whose expression within a cell, e.g., a cell modified as described herein, enables the cell to inhibit or evade immune rejection after transplant or engraftment into a subject. In some embodiments, the protein or polypeptide is HLA-E, HLA-G, CTL4, CD47, or an associated ligand.

In some embodiments, the nucleic acid sequence of interest may encode a T cell receptor (TCR) or an antigen-binding fragment thereof, e.g., a recombinant TCR. In some embodiments, the recombinant TCR can bind to an antigen of interest, e.g., an antigen selected from, but not limited to, CD279, CD2, CD95, CD152, CD223CD272, TIM3, KIR, A2aR, SIRPa, CD200, CD200R, CD300, LPA5, NY-ESO, PD1, PDL1, or MAGE-A3/A6. In some embodiments, the TCR or antigen-binding fragment thereof can bind to a viral antigen, e.g., an antigen from hepatitis A, hepatitis B, hepatitis C (HCV), human papilloma virus (HPV) (e.g., HPV-16 (such as HPV-16 E6 or HPV-16 E7), HPV-18, HPV-31, HPV-33, or HPV-35), Epstein-Barr virus (EBV), human herpes virus 8 (HHV-8), human T-cell leukemia virus-1 (HTLV-1), human T-cell leukemia virus-2 (HTLV-2) or a cytomegalovirus (CMV).

In some embodiments, the nucleic acid sequence of interest may encode a single-chain variable fragment that can bind to CD47, PD1, CTLA4, CD28, OX40, 4-1BB, and ligands thereof.

As used herein, the term “HLA-G” refers to the HLA non-classical class I heavy chain paralogues. This class I molecule is a heterodimer consisting of a heavy chain and a light chain (beta-2 microglobulin). The heavy chain is anchored in the membrane. HLA-G is expressed on fetal derived placental cells. HLA-G is a ligand for NK cell inhibitory receptor KIR2DL4, and therefore expression of this HLA by the trophoblast defends it against NK cell-mediated death. See e.g., Favier et al., PLOS One 2011 6(7):e21011, the entire contents of which are incorporated herein by reference. An exemplary sequence of HLA-G is set forth as NG_029039.1.

As used herein, the term “HLA-E” refers to the HLA class I histocompatibility antigen, alpha chain E, also sometimes referred to as MHC class I antigen E. The HLA-E protein in humans is encoded by the HLA-E gene. The human HLA-E is a non-classical MHC class I molecule that is characterized by a limited polymorphism and a lower cell surface expression than its classical paralogues. This class I molecule is a heterodimer consisting of a heavy chain and a light chain (beta-2 microglobulin). The heavy chain is anchored in the membrane. HLA-E binds a restricted subset of peptides derived from the leader peptides of other class I molecules. HLA-E expressing cells escape allogeneic responses and lysis by NK cells. See, e.g., Gornalusse et al., Nature Biotechnology 2017 35(8): 765-772, the entire contents of which are incorporated herein by reference. Exemplary sequences of the HLA-E protein are provided in NM_005516.6.

As used herein, the term “CD47,” also sometimes referred to as “integrin associated protein” (IAP), refers to a transmembrane protein that in humans is encoded by the CD47 gene. CD47 belongs to the immunoglobulin superfamily, partners with membrane integrins, and also binds the ligands thrombospondin-1 (TSP-1) and signal-regulatory protein alpha (SIRPa). CD47 acts as a signal to macrophages that allows CD47-expressing cells to escape macrophage attack. See, e.g., Deuse et al., Nature Biotechnology 2019 37:252-258, the entire contents of which are incorporated herein by reference.

In some embodiments, the nucleic acid sequence of interest may encode a chimeric switch receptor (see, e.g., WO2018094244A1; Ankri et al., Journal of Immunology 2013 191:4121-4129; Roth et al., Cell. 2020 181(3):728-744.e21; and Boyerinas et al., Blood, 2017 130(S1):1911). In some embodiments, chimeric switch receptors are engineered cell-surface receptors comprising an extracellular domain from an endogenous cell-surface receptor and a heterologous intracellular signaling domain, such that ligand recognition by the extracellular domain results in activation of a different signaling cascade than that activated by the wild-type form of the cell-surface receptor. In some embodiments, a chimeric switch receptor comprises an extracellular domain of an inhibitory cell-surface receptor fused to an intracellular domain that leads to the transmission of an activating signal rather than the inhibitory signal normally transduced by the inhibitory cell-surface receptor. In some embodiments, extracellular domains derived from cell-surface receptors known to inhibit immune effector cell activation can be fused to activating intracellular domains. In such an embodiment, engagement of the corresponding ligand may then activate signaling cascades that increase, rather than inhibit, the activation of the immune effector cell. For example, in some embodiments, a gene product of interest is a PD1-CD28 switch receptor, wherein the extracellular domain of PD1 is fused to the intracellular signaling domain of CD28 (see, e.g., Liu et al., Cancer Res 76:6 (2016), 1578-1590 and Moon et al., Molecular Therapy 22 (2014), S201). In some embodiments, encoding gene product of interest is or comprises the extracellular domain of CD200R and the intracellular signaling domain of CD28 (see, e.g., Oda et al., Blood 130:22 (2017), 2410-2419).

In some embodiments, the nucleic acid sequence of interest may encode a reporter (e.g., GFP, mCherry, etc.). In certain embodiments, a reporter may be a colored or fluorescent protein such as: blue/UV proteins, e.g., TagBFP, mTagBFP2, Azurite, EBFP2, mKalamal, Sirius, Sapphire, T-Sapphire; cyan proteins, e.g. ECFP, Cerulean, SCFP3A, mTurquoise, mTurquoise2, monomeric Midoriishi-Cyan, TagCFP, mTFP1; green proteins, e.g. EGFP, Emerald, Superfolder GFP, Monomeric Azami Green, TagGFP2, mUKG, m Wasabi, Clover, mNeonGreen; yellow proteins, e.g. EYFP, Citrine, Venus, SYFP2, TagYFP; orange proteins, e.g., Monomeric Kusabira-Orange, mKOK, mK02, mOrange, mOrange2; red proteins, e.g., mRaspberry, mStrawberry, mTangerine, tdTomato, TagRFP, TagRFP-T, mApple, mRuby, mRuby2; far-red proteins, e.g. mPlum, HcRed-Tandem, mKate2, mNeptune, NirFP; near-IR proteins, e.g. TagRFP657, IFP1.4, iRFP; long stokes shift proteins, e.g., mKeima Red, LSS-mKate1, LSS-mKate2, mBeRFP; photoactivatible proteins, e.g. PA-GFP, PAmCherryl, PATagRFP; photoconvertible proteins, e.g., Kaede (green), Kaede (red), KikGRI (green), KikGRI (red), PS-CFP2, PS-CFP2, mEos2 (green), mEos2 (red), mEos3.2 (green), mEos3.2 (red), PSmOrange, PSmOrange, photoswitchable proteins, e.g., Dronpa, and combinations thereof.

In some embodiments, the nucleic acid sequence of interest may be a suicide gene (see e.g., Zarogoulidis et al., J Genet Syndr Gene Ther. 2013 4:1000139). In some embodiments, a suicide gene can use a gene-directed enzyme prodrug therapy (GDEPT) approach, a dimerization inducing approach, and/or therapeutic monoclonal antibody mediated approach. In some embodiments, a suicide gene is biologically inert, has an adequate bio-availability profile, an adequate bio-distribution profile, and can be characterized by intrinsic acceptable and/or absence of toxicity. In some embodiments, a suicide gene codes for a protein able to convert, at a cellular level, a non-toxic prodrug into a toxic product. In some embodiments, a suicide gene may improve the safety profile of a cell described herein (see e.g., Greco et al., Front Pharmacology 2015 6:95; Jones et al., Front Pharmacology 2014 5:254). In some embodiments, a suicide gene is a herpes simplex virus thymidine kinase (HSV-TK). In some embodiments, a suicide gene is a cytosine deaminase (CD). In some embodiments, a suicide gene is an apoptotic gene (e.g., a caspase). In some embodiments, a suicide gene is dimerization inducing, e.g., comprising an inducible FAS (iFAS) or inducible Caspase9 (iCasp9)/AP1903 system. In some embodiments, a suicide gene is a CD20 antigen, and cells expressing such an antigen can be eliminated by clinical-grade anti-CD20 antibody administration. In some embodiments, a suicide gene is a truncated human EGFR polypeptide (huEGFRt) which confers sensitivity to a pharmaceutical-grade anti-EGFR monoclonal antibody, e.g., cetuximab. In some embodiments a suicide gene is a c-myc tag, which confers sensitivity to pharmaceutical-grade anti-c-myc antibodies.

In some embodiments, the nucleic acid sequence of interest may be a safety switch signal. In cell therapy, a safety switch can be used to stop proliferation of the genetically modified cells when their presence in the patient is not desired, for example, if the cells do not function properly, if planned therapeutic interventions change, or if the therapeutic goal has been achieved. In some embodiments, a safety switch may, for example, be a so-called suicide gene, or suicide switch, which upon administration of a pharmaceutical compound to the patient, will be activated or inactivated such that the cells enter apoptosis. Suicide genes, sometimes called suicide switches or safety switches can be triggered or activated by a cellular event, environmental event or chemical agent resulting in a cellular response by cells that have the suicide gene incorporated in their genome. In some embodiments, activation of a safety switch induces cellular apoptosis. In some embodiments, activation of the safety switch inhibits growth of cells incorporated with the safety switch. In some embodiments, a suicide switch may encode an enzyme not found in humans (e.g., a bacterial or viral enzyme) that converts a harmless substance into a toxic metabolite in the human cell. Examples of suicide switch include, without limitation, genes for thymidine kinases, cytosine deaminases, intracellular antibodies, telomerases, toxins, caspases (e.g., iCaspase9) and HSV-TK, and DNases. In some embodiments, the suicide gene may be a thymidine kinase (TK) gene from the Herpes Simplex Virus (HSV) and the suicide TK gene becomes toxic to the cell upon administration of ganciclovir, valganciclovir, famciclovir, or the like to the patient.

In some embodiments, a safety switch may be a rapamycin-inducible human Caspase 9-based (RapaCasp9) cellular suicide switch in which a truncated caspase 9 gene, which has its CARD domain removed, is linked after either the FRB (FKBP12-rapamycin binding) domain of mTOR, or FKBP12 (FK506-binding protein 12). Addition of the drug rapamycin enables heterodimerization of FRB and FKBP12 which subsequently causes homodimerization of truncated caspase 9 and induction of apoptosis. In some embodiments, using a two construct and/or biallelic approach as described herein, FRB and FKBP12 are separated onto different alleles by incorporating two donor constructs, one with one or more transgenes plus FRB, the other with one or more transgenes plus FKBP12. When referring to a safety switch in this application, it should be interpreted to include all components necessary for the function of the safety switch (e.g., FRB domain and FKBP12 domain and truncated caspase 9 gene are all components of, and make up, the safety switch).

### Methods of Treatment

The present disclosure, among other things, provides methods and LSRs that can be used in the treatment of a disease, disorder, or condition. In some embodiments, LSRs described herein can be used to integrate a gene of interest, including but limited to, those described herein for the treatment of a subject. In some embodiments, LSRs as described herein can be used for ex vivo modification of a cell. In some embodiments, the cell is a mammalian cell. In some embodiments, the mammalian cell is a human cell. In some embodiments, the human cell is derived from the subject, e.g., an autologous cell. In some other embodiments, the human cell is derived from an individual that is not the subject, e.g., an allogeneic cell. In some embodiments, the ex vivo modified cells are administered to a subject as a pharmaceutical composition. In some other embodiments, the LSRs of the present disclosure are administered in vivo to a subject as a pharmaceutical composition.

Administration of a pharmaceutical compositions described herein may be carried out in any convenient manner (e.g., injection, ingestion, transfusion, inhalation, implantation, or transplantation). In some embodiments, a pharmaceutical composition described herein is administered by injection or infusion. Pharmaceutical compositions described herein may be administered to a subject intravenously, transarterially, subcutaneously, intradermally, intratumorally, intranodally, intramedullary, intramuscularly, or intraperitoneally. In some embodiments, a pharmaceutical composition described herein is administered parenterally (e.g., intravenously, subcutaneously, intraperitoneally, or intramuscularly). In some embodiments, a pharmaceutical composition described herein is administered by intravenous infusion or injection. In some embodiments, a pharmaceutical composition described herein is administered by intramuscular or subcutaneous injection.

In some embodiments, a pharmaceutical composition described herein is administered at a pharmaceutically suitable dosage to a subject. In some embodiments, a pharmaceutical composition described herein is administered monthly. In some embodiments, a pharmaceutical composition described herein is administered once every other month. In some embodiments, a pharmaceutical composition described herein is administered once every three months. In some embodiments, a pharmaceutical composition described herein is administered once every six months. In some embodiments, a pharmaceutical composition described herein is administered once a year.

### EXAMPLES

### Example 1: Identification of Large Serine Recombinases and Uses Thereof

The present Example describes computational methods that were used to assess phage insertions and identify cognate large serine recombinases from thousands of bacterial genomes, and find and characterize the respective potential attachment sites in the human genome (attH) for these recombinases. As described herein, these methods allowed for the identification and assessment of the novel large serine recombinases of Table 1 and their respective potential attachment sites in the human genome. The application of these novel large serine recombinases allows for efficient and specific integration of exogenous nucleic acid, e.g., exogenous DNA into a host human genome.

Computational Discovery of Phage Insertions from Thousands of Bacterial Genomes

Genomes from numerous bacterial isolates from within the same species were compared against each other in order to detect putative phage insertions. Bacterial genomes were downloaded from the NCBI Refseq database and a collection of bacterial genomes in the ENA database (available through the world wide web at ftp.ebi.ac.uk/pub/databases/ENA2018-bacteria-661k/). Data analysis was performed separately for the NCBI and ENA datasets. Bacterial species with at least two genome assemblies in either dataset were used for analysis. Overall, 283,589 genome assemblies from the NCBI Refseq database and 635,246 genome assemblies from the ENA database were evaluated. The genome assemblies of each bacterial species were grouped by their respective NCBI taxon ID.

In order to compare the genomes of the same bacterial species, the most complete genome was selected as a reference and then aligned to shortened sequences (also known as reads) that were generated from the other, less complete genomes available for the species. For the NCBI dataset, the evaluation of genome assemblies was based on the assembly status with the following ranking: Complete>Chromosome>Scaffold>Contig and assembly size, while the ENA genome assemblies were ranked by the genome completeness scores provided by the dataset. For bacterial species that have more than one distantly related lineage, one reference genome was selected from each lineage for separate analysis. The computational tool PopPunk was used to estimate the core genome distances among genomes (Lees et al. 2019), and genome assemblies within 0.05 core genome distance were grouped into one lineage. Non-reference genomes were each tiled into 300 bp long sequences, with 100 bp overlaps. Each of these sequences were converted into reads and assembled into FASTQ file format. These non-reference genome reads were aligned using BWA MEM algorithm (Li and Durbin 2009).

The putative phage insertions were identified based on either of two read alignment patterns. The first pattern assumes that the reference bacterial genome does not contain a phage insertion. As such, reads generated from the phage-bacterial genome boundary in a genome containing the phage insertion would be aligned to the attB site in the reference genome with one end being clipped (including both soft-clipped and hard-clipped ends). A genomic region supported by clipped reads in both forward and reverse directions was considered to be a putative phage insertion site, and the full phage insertion sequence was inferred from the positions of clipped reads in their source genome. Alternatively, in a second pattern, assuming a phage insertion is present in the reference genome, reads generated from genomes without the phage insertion would be split to align the two flanking regions outside the phage insertion (e.g., the left and right ends are aligned with some distance). This is known as a “split read”. As a result, the full phage insertion sequence can be determined to be the sequence between the two aligned positions of the “split read” in the reference genome.

### Identification of Large Serine Recombinases and Their Cognate Attachment Sites in Bacterial Genomes

The identified putative phage insertions exemplified in Table 1 were analyzed using the gene prediction software of Prodigal (PROkaryotic DYnamic programming Gene-finding ALgorithm) (Hyatt et al. 2010) to identify protein coding sequences. These sequences were analyzed using the HMMR computer software package (Eddy 2009) to identify the three domains typically associated with large serine recombinases (protein domains in Table 1): a resolvase/invertase domain (PF00239), a zinc ribbon domain (PF13408), and a recombinase domain Pfam (PF07508). Predicted recombinase proteins with at least one of these three domains were retained for further analysis.

The cognate attachment sites (attP/B) of each large serine recombinase were reconstructed from the sequences surrounding the phage insertion boundary. The sequences flanking outside a phage insertion were concatenated to generate an attB sequence, B₁+D+B₂. Moreover, the sequences inside of a phage insertion were concatenated to generate an attP sequence, P₂+D+P₁. D represents the conserved sequences (about 2-20 bp) shared between sequences in the left and right boundary of a phage element, which is also called target site duplication generated by phage insertion. The center core dinucleotide in attB/attP was further determined by searching for the position within D that achieves the optimal alignment between the attP left half-site sequence and the reverse complement of its right half-site sequence (considering the greater symmetry of the attP sequence). Finally, the attP and attB sequences, ideally with the same core dinucleotides in the center, were reconstructed as 50 bp sequences and 40 bp sequences, respectively.

### Selection Criteria for High-Quality Large Serine Recombinase Candidates

First, in order to arrive at the novel set of large serine recombinases in Table 1, several filtering criteria were applied to select a subset of high-quality candidates and their respective attB/P sites. First, the size of phage insertions was restricted to approximately 3-200 kb. Second, the distance from the LSR protein sequence to the phage insertion boundary had to be within 500 bp. Third, target site duplication (D) had to be in the range of 2-20 bp. Fourth, only LSR proteins containing at least two of the three canonical LSR protein domains or ones comprising 400-700 unambiguous amino acids were retained. To remove redundant large serine recombinases with the same attB and attP sites identified in different isolates or bacterial species, only one large serine recombinase and their respective attB and attP sites was retained as a representative in Table 1.

Second, in order to identify putative large serine recombinases more likely capable of mediating recombination with the human genome, the attB and attP sequences of each large serine recombinase were searched against a human reference genome (hg38) using CALITAS (Fennell et al. 2021) not allowing for gaps in the alignment. For each LSR, the attP sequence is 10-bp larger than its corresponding attB sequence, so the potential 5-bp linker region at each attP half site (the sequence between the ZD and RD motifs; FIG. 2) was masked with NNNNN, so that mismatches between the sequences in the linker region and the corresponding human region would not be counted as mismatches. The center dinucleotide in both attB and attP was also masked with NN, since it can be changed to any bases that match the corresponding human sites. For each large serine recombinase, the best alignment with the fewest mismatches was selected from all attB and attP matched sequences, and the best matched human sequence is described as attH (potential attachment site in human genome). The attB or attP sequence of each large serine recombinase used to align with attH (and most closely matches attH) is termed attA, and the other attachment site sequence (either the attB or attP sequence with the center dinucleotides changed to match attH) is termed attD (donor sequence that can be used for targeted integration at an attH). Finally, alignment between attA and attH was refined using CALITAS (Fennell et al. 2021) to determine the number of mismatches and gaps between the two sequences.

### Categorization of Identified Large Serine Recombinases

The present disclosure describes a novel set of large serine recombinases and their respective predicted attachment sites in the human genome that allow for efficient genetic manipulation and integration of large DNA payloads. As described herein, these large serine recombinase systems have been discovered through the development and use of computational algorithms to analyze a large number of bacterial genomes for recombinase-mediated phage insertions, and then comparison of the predicted recombinase attachment site sequences in the bacteria and phage genomes to similar sequences found in the human genome. This library of large serine recombinases and cognate human attachment sites are disclosed in Table 1.

Table 1 is organized with priority given to the large serine recombinase systems with lowest calculable mismatches (mm) between the attachment site sequence (attA sequence, being whichever of the attB or attP sequence that most closely matches the attH sequence) and human attachment site sequence (attH sequence), using CALITAS as described above. These large serine recombinases are numbered accordingly under system ID (system_id) up through the 12,713 identified. These high-quality large serine recombinase candidates were identified from different bacterial genomes as described above, and are annotated within Table 1 with the bacterial species name (species_name) and associated respective NCBI taxon id (taxon_id) with their isolate accession number (isolate_accession). Computational identification of putative phage insertion is further described within this table as where the insertion would occur (insertion_origin), its size (insertion_size), and location within the large serine recombinase origin (lsr_location).

All LSRs are further defined by the strand of the large serine recombinase (lsr_strand) and respective protein sequence (lsr_protein). The sequences of the predicted attachment sites for integration, attH, with the fewest mismatches based on sequence alignment with either attB/attP for each corresponding large serine recombinase are described in Table 1. The human genomic locations of these attH sites are further defined by their respective chromosome number, nucleic acid start position and nucleic acid end position (attH_coordinates) of the predicted insertion site in a respective DNA strand (sense, + or antisense, −). For certain LSRs, Table 1 also includes the human genomic locations of other potential attachment sites for integration (alt_attH_sites). In some embodiments, these alternative attH sites include the same number of mismatches as the attH site described above (based on sequence alignment with either attB/attP for each corresponding large serine recombinase). In some embodiments, these alternative attH sites include additional mismatches based on sequence alignment with either attB/attP for each corresponding large serine recombinase.

For each system ID in Table 1 (i.e., each row of Table 1), there are SEQ ID NOs identified by each of the following headers: “LSR_Protein SEQ ID NO:”, “attp_sequence SEQ ID NO:”, “attb_sequence SEQ ID NO:”, “attD_sequence SEQ ID NO:”, and “attH_sequence SEQ ID NO:”. The SEQ ID NOs in Table 1 serve as placeholders for the sequences identified as SEQ ID NOs: 1-63565 in the Sequence Listing. As used herein, “sequence selected from Table 1” and similar terms are understood to refer to the sequences in the Sequence Listing identified by the SEQ ID NOs in Table 1.

### Example 2: Screening of Large Serine Recombinases

The present Example describes methods (Individual LSR Screening) that were used to assess the functionality of some individual LSRs identified in Table 3. The present Example also describes methods (Pooled LSR Screening) that were used to assess the functionality of cluster representative LSRs identified in Table 2.

### Individual LSR Screening

### Synthesis and Cloning

Each mammalian codon-optimized LSR gene was synthesized downstream of its respective 40 bp attB sequence and cloned via Gibson assembly into an expression plasmid which contained a 5′ promoter and 3′ P2A-GFP expression cassette. This cloning process was automated via BioXP 3250 (CODEX DNA). The attP sequence was synthesized as an oligonucleotide (IDT) and cloned using NEBridgeR Golden Gate Assembly Kit (NEB) upstream a promoter-less mCherry gene.

### Preparation and Sequencing

Assembled plasmids were transformed into OneShotTop10 Bacteria or c3040H competent cells (NEB) and plated onto agar plates with appropriate antibiotics. Colonies with growth were picked and grown in 1.5 mL of LB selection media overnight and finally miniprepped with Qiagen Plasmid Plus 96 Miniprep kit (Qiagen). The isolated plasmid preps were sequenced via Oxford Nanopore Sequencing to validate cloning.

### Plasmid Recombination Assay

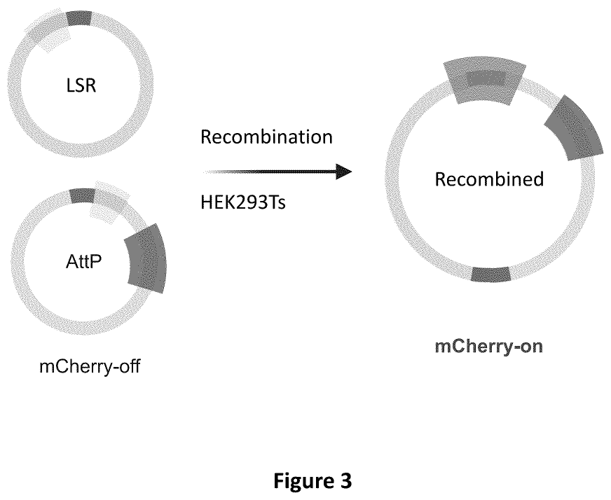

For screening of individual recombinase function in mammalian cells, each attB-LSR plasmid and an attP-mCherry plasmid were co-transfected into HEK-293T cells in a 96 well format using TransIT-293 Transfection Reagent (Mirus) (see FIG. 3). Two control groups were used per LSR: an attP-mCherry plasmid alone to quantify background expression, and attB-LSR with a non-specific mCherry to assess cross-reactivity of recombination. After 48-72 hours of culture, the cells were trypsinized and pelleted. Half were re-suspended and analyzed for mCherry protein (PE-Texas Red) and eGFP protein (FITC) expression via flow cytometry (Novocyte Quanteon Flow Cytometer System). Mean fluorescent intensity (MFI) of PE-Texas Red was used as the readout for recombination with eGFP as a surrogate for LSR expression. Fluorescent data was normalized by dividing the MFI of the recombination group by the MFI of the promoterless attP-mCherry only group to determine fold increase in mCherry fluorescence caused by promoter-swapping. With the remaining half of the cell population, genomic DNA was isolated using DNAdvance Kit (Beckman Coulter) and a ddPCR reaction was subsequently performed to quantify the percent recombination (BioRad: ddPCR Supermix for Probes). 2 ddPCR assays were designed; one measuring an amplicon across the recombination junction in a recombined plasmid and the other measuring mCherry (IDT). The ratio of recombination junction positive droplets to mCherry droplets was then used to calculate percent recombination. The ddPCR data, after determining recombination positive droplets, was normalized to % recombination of Bxb1, a consistent and highly active LSR in the field, which was a control present on each transfection and instrument run. Empty data points represent lost replicate plates due to instrument or user error.

### Results

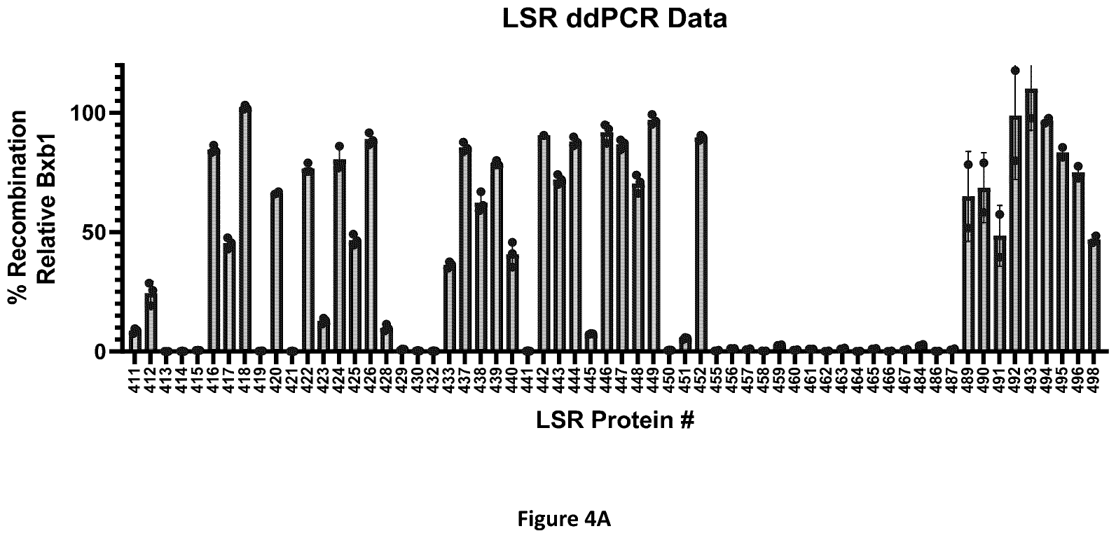

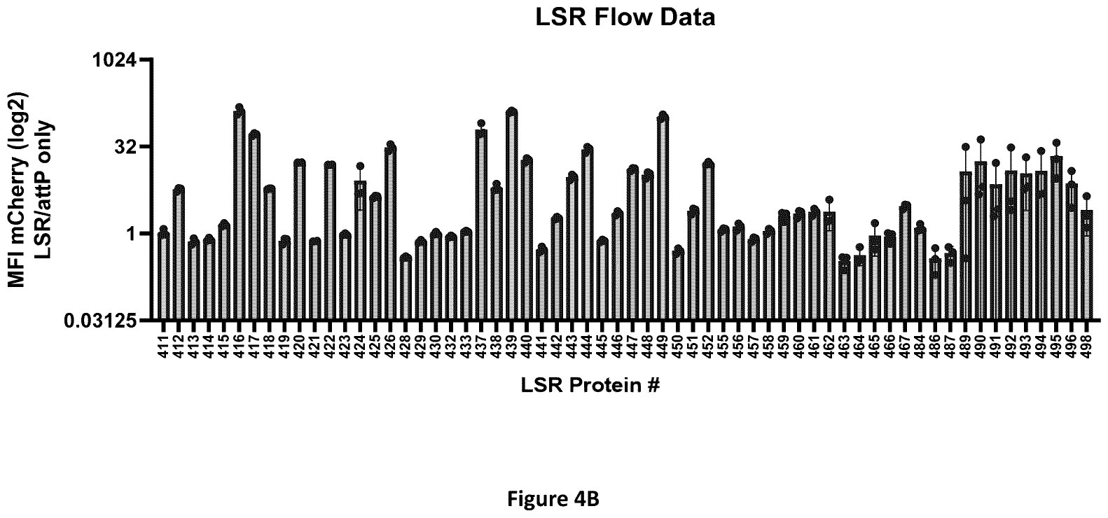

Many LSRs that were tested showed recombinase activity, as seen by positive % recombination relative to Bxb1 by ddPCR (FIG. 4A) and MFI mCherry when viewing the fold increase relative to promoterless mCherry (attP only, FIG. 4B). These results showed that more than half of the screened LSRs have above 2% recombination activity relative to Bxb1 and greater than 2-fold increase in MFI of mCherry relative to promoterless mCherry. Notably, the ddPCR and mCherry MFI results showed a strong correlation. Table 3 provides details for the individual LSRs that were tested in accordance with these methods and also notes the cluster they belong to (see Pooled LSR Screening below).

TABLE 3
LSRs from Individual LSR Screening and Inclusion in LSR Clusters

| | | LSR | | | | | | |
|---|---|---|---|---|---|---|---|---|
| System | | Protein | attP | attB | attD | attH | | Screened |
| ID: | LSR location | SEQ ID NO: | SEQ ID NO: | SEQ ID NO: | SEQ ID NO: | SEQ ID NO: | Cluster | Label |
| 1406 | SEYX01000017.1: 32210-33946 | 7026 | 7027 | 7028 | 7029 | 7030 | 199 | PRO411 |
| 1408 | JAGDLG010000002.1: 43469-45241 | 7036 | 7037 | 7038 | 7039 | 7040 | 199 | PRO412 |
| 765 | CDMF01000001.1: 3566594-3568282 | 3821 | 3822 | 3823 | 3824 | 3825 | 2746 | PRO413 |
| 62 | UTAC01000001.1: 161628-163097 | 306 | 307 | 308 | 309 | 310 | 1119 | PRO414 |
| 55 | CP012312.1: 2083693-2085372 | 271 | 272 | 273 | 274 | 275 | 237 | PRO415 |
| 11045 | SAMN06040332.contig00014: 98555-100075 | 55221 | 55222 | 55223 | 55224 | 55225 | 7 | PRO416 |
| 1529 | NVDH01000013.1: 413916-415436 | 7641 | 7642 | 7643 | 7644 | 7645 | 106 | PRO417 |
| 4671 | VTTT01000003.1: 200824-202329 | 23351 | 23352 | 23353 | 23354 | 23355 | 528 | PRO418 |
| 169 | CTKJ01000021.1: 42840-44573 | 841 | 842 | 843 | 844 | 845 | 115 | PRO419 |
| 166 | QSLI01000006.1: 62456-63892 | 826 | 827 | 828 | 829 | 830 | 387 | PRO420 |
| 5517 | NTRM01000007.1: 108739-110214 | 27581 | 27582 | 27583 | 27584 | 27585 | 45 | PRO421 |
| 917 | CP047394.1: 2957116-2958642 | 4581 | 4582 | 4583 | 4584 | 4585 | 1823 | PRO422 |
| 668 | DS264311.1: 17878-19620 | 3336 | 3337 | 3338 | 3339 | 3340 | 2755 | PRO423 |
| 4670 | JADWNC010000007.1: 204939-206444 | 23346 | 23347 | 23348 | 23349 | 23350 | 528 | PRO424 |
| 1936 | VWSY01000001.1: 2767353-2768864 | 9676 | 9677 | 9678 | 9679 | 9680 | 25 | PRO425 |
| 2015 | JACBEG010000001.1: 430924-432438 | 10071 | 10072 | 10073 | 10074 | 10075 | 695 | PRO426 |
| 2393 | LVUK01000124.1: 1052-2899 | 11961 | 11962 | 11963 | 11964 | 11965 | 24 | PRO427 |
| 11979 | SAMN00254032.contig00004: 162231-163994 | 59891 | 59892 | 59893 | 59894 | 59895 | 34 | PRO428 |
| 4606 | JACYXR010000011.1: 190394-192022 | 23026 | 23027 | 23028 | 23029 | 23030 | 298 | PRO429 |
| 4294 | JTMO01000027.1: 80289-81905 | 21466 | 21467 | 21468 | 21469 | 21470 | 147 | PRO430 |
| 11134 | RBSL01000205.1: 16360-18030 | 55666 | 55667 | 55668 | 55669 | 55670 | 188 | PRO431 |
| 348 | JYLP01000027.1: 19858-21285 | 1736 | 1737 | 1738 | 1739 | 1740 | 263 | PRO432 |
| 2192 | RYCU01000001.1: 643473-645272 | 10956 | 10957 | 10958 | 10959 | 10960 | 64 | PRO433 |
| 1084 | AIDX01000001.2: 1656567-1658024 | 5416 | 5417 | 5418 | 5419 | 5420 | 117 | PRO437 |
| 11584 | FVFC01000006.1: 167188-168609 | 57916 | 57917 | 57918 | 57919 | 57920 | 101 | PRO438 |
| 883 | NUQZ01000052.1: 68993-70510 | 4411 | 4412 | 4413 | 4414 | 4415 | 1356 | PRO439 |
| 828 | CP068488.1: 4213722-4215398 | 4136 | 4137 | 4138 | 4139 | 4140 | 72 | PRO440 |
| 6848 | SAMEA3545244.contig00001: 110539-112131 | 34236 | 34237 | 34238 | 34239 | 34240 | 87 | PRO441 |
| 1483 | CZAV01000001.1: 649554-650777 | 7411 | 7412 | 7413 | 7414 | 7415 | 2008 | PRO442 |
| 1689 | CP016349.1: 1998207-2000111 | 8441 | 8442 | 8443 | 8444 | 8445 | 418 | PRO443 |
| 2686 | JABEQB010000025.1: 3462-4988 | 13426 | 13427 | 13428 | 13429 | 13430 | 2784 | PRO444 |
| 767 | BBIV01000008.1: 75641-77248 | 3831 | 3832 | 3833 | 3834 | 3835 | 2775 | PRO445 |
| 1216 | JAAQXZ010000018.1: 98061-99626 | 6076 | 6077 | 6078 | 6079 | 6080 | 1622 | PRO446 |
| 1385 | CP049698.1: 2416186-2418009 | 6921 | 6922 | 6923 | 6924 | 6925 | 2003 | PRO447 |
| 88 | JRFS01000048.1: 2943-4670 | 436 | 437 | 438 | 439 | 440 | 100 | PRO448 |
| 428 | LDGR01000022.1: 239790-241181 | 2136 | 2137 | 2138 | 2139 | 2140 | 178 | PRO449 |
| 5652 | CAKAFH0100000011: 613414-614679 | 28256 | 28257 | 28258 | 28259 | 28260 | 545 | PRO450 |
| 12187 | JACEVK010000003.1: 94997-96499 | 60931 | 60932 | 60933 | 60934 | 60935 | 236 | PRO451 |
| 7621 | JACRTO010000008.1: 76315-77868 | 38101 | 38102 | 38103 | 38104 | 38105 | 250 | PRO452 |

### Pooled LSR Screening

### Clustering and Design

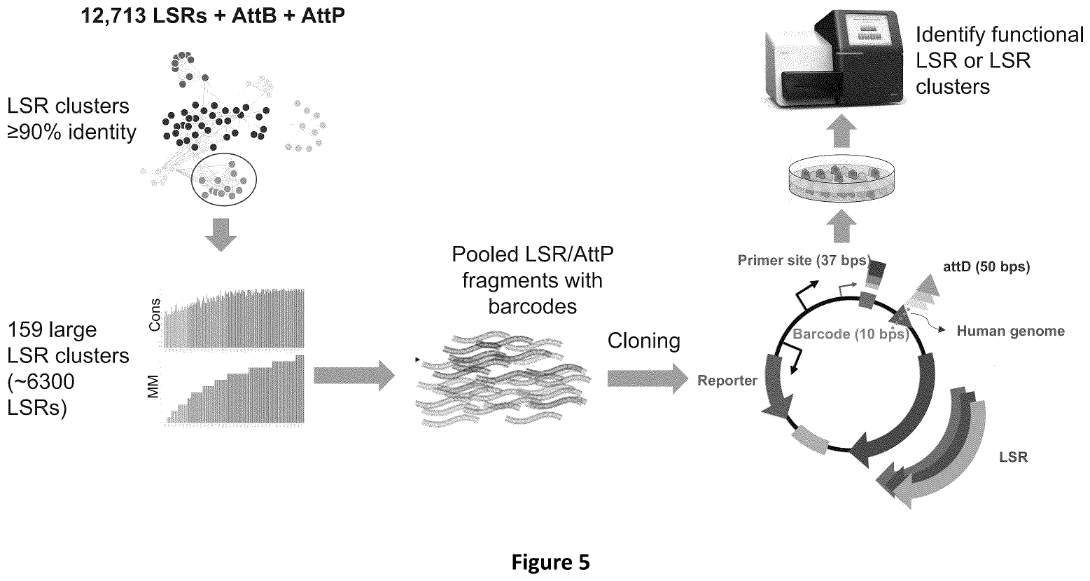

As shown in FIG. 5, starting from the 12,713 identified LSR proteins we selected 12,003 that contained each of a resolvase/invertase domain (PF00239), zinc ribbon domain (PF13408), and recombinase domain (PF07508) and clustered them based on ≥90% sequence identity across the three protein domains using the UCLUST algorithm (Edgar 2010). 159 large LSR clusters each containing at least 10 individual LSR proteins were retained for future analysis. These 159 clusters comprised 6,280 LSRs in total. The individual LSR that is closest in terms of genetic distance to all other individual LSRs within the same cluster (the centroid LSR) was selected as the cluster representative LSR for further screening. Table 2 depicts the representative LSR for each of the 159 clusters.

TABLE 2
Representative LSRs from LSR Clusters

| | | LSR | | | | | |
|---|---|---|---|---|---|---|---|
| System | | Protein | attP | attB | attD | attH | Cluster |
| ID: | LSR location | SEQ ID NO: | SEQ ID NO: | SEQ ID NO: | SEQ ID NO: | SEQ ID NO: | NO: |
| 6023 | SAMEA4426195.contig00019: 60060-61580 | 30111 | 30112 | 30113 | 30114 | 30115 | 1 |
| 11786 | SAMEA4559502.contig00002: 272767-274290 | 58926 | 58927 | 58928 | 58929 | 58930 | 2 |
| 2123 | SAMN02847255.contig00006: 127364-129007 | 10611 | 10612 | 10613 | 10614 | 10615 | 3 |
| 1548 | SAMEA4816500.contig00002: 535421-536779 | 7736 | 7737 | 7738 | 7739 | 7740 | 4 |
| 10695 | SAMN04497704.contig00023: 12393-13916 | 53471 | 53472 | 53473 | 53474 | 53475 | 5 |
| 6605 | SAMN04357335.contig00009: 180468-182090 | 33021 | 33022 | 33023 | 33024 | 33025 | 6 |
| 8039 | SAMEA4548080.contig00004: 197458-198978 | 40191 | 40192 | 40193 | 40194 | 40195 | 7 |
| 9840 | SAMEA1031511.contig00009: 3354-5123 | 49196 | 49197 | 49198 | 49199 | 49200 | 8 |
| 9156 | SAMEA1031428.contig00011: 70731-72380 | 45776 | 45777 | 45778 | 45779 | 45780 | 9 |
| 407 | SAMEA3916543.contig00008: 34962-36575 | 2031 | 2032 | 2033 | 2034 | 2035 | 10 |
| 1137 | SAMEA1026767.contig00005: 68407-69852 | 5681 | 5682 | 5683 | 5684 | 5685 | 11 |
| 7247 | CP031643.1: 3713651-3715198 | 36231 | 36232 | 36233 | 36234 | 36235 | 12 |
| 8890 | ABAB01000021.1: 9995-11602 | 44446 | 44447 | 44448 | 44449 | 44450 | 13 |
| 6969 | SAMEA4560321.contig00018: 78514-80079 | 34841 | 34842 | 34843 | 34844 | 34845 | 14 |
| 9998 | SAMEA2053924.contig00007: 72430-74199 | 49986 | 49987 | 49988 | 49989 | 49990 | 15 |
| 1982 | LT969517.1: 1729777-1731399 | 9906 | 9907 | 9908 | 9909 | 9910 | 16 |
| 8471 | SAMEA102223918.contig00007: 26330-27757 | 42351 | 42352 | 42353 | 42354 | 42355 | 17 |
| 10474 | SAMN03197368.contig00004: 83225-84601 | 52366 | 52367 | 52368 | 52369 | 52370 | 18 |
| 379 | SAMN07159041.contig00003: 161292-162821 | 1891 | 1892 | 1893 | 1894 | 1895 | 19 |
| 9245 | AVHW01000071.1: 1586-3184 | 46221 | 46222 | 46223 | 46224 | 46225 | 20 |
| 12340 | SAMN09062737.contig00010: 80709-82280 | 61696 | 61697 | 61698 | 61699 | 61700 | 21 |
| 10432 | SAMN08922688.contig00004: 147964-149580 | 52156 | 52157 | 52158 | 52159 | 52160 | 22 |
| 3941 | SAMEA1034821.contig00012: 10790-12544 | 19701 | 19702 | 19703 | 19704 | 19705 | 23 |
| 4183 | SAMEA882193.contig00023: 13310-15157 | 20911 | 20912 | 20913 | 20914 | 20915 | 24 |
| 8653 | CP021422.1: 1211528-1213039 | 43261 | 43262 | 43263 | 43264 | 43265 | 25 |
| 2512 | SAMEA1564953.contig00005: 111328-112821 | 12556 | 12557 | 12558 | 12559 | 12560 | 26 |
| 1279 | SAMEA4061524.contig00047: 12618-14162 | 6391 | 6392 | 6393 | 6394 | 6395 | 27 |
| 4096 | LEER01000007.1: 162255-164171 | 20476 | 20477 | 20478 | 20479 | 20480 | 28 |
| 2495 | SAMEA3539452.contig00050: 9635-11257 | 12471 | 12472 | 12473 | 12474 | 12475 | 29 |
| 8444 | CP062497.1: 2600356-2601858 | 42216 | 42217 | 42218 | 42219 | 42220 | 30 |
| 2493 | SAMN02923806.contig00001: 246631-248457 | 12461 | 12462 | 12463 | 12464 | 12465 | 31 |
| 2204 | SAMEA3484564.contig00031: 12997-14862 | 11016 | 11017 | 11018 | 11019 | 11020 | 32 |
| 12219 | JAKNFY010000003.1: 230362-232218 | 61091 | 61092 | 61093 | 61094 | 61095 | 33 |
| 11980 | CAAGXG010000001.1: 937537-939300 | 59896 | 59897 | 59898 | 59899 | 59900 | 34 |
| 11265 | SAMEA103957246.contig00028: 13053-14663 | 56321 | 56322 | 56323 | 56324 | 56325 | 35 |
| 4213 | SAMEA103956214.contig00005: 128521-129966 | 21061 | 21062 | 21063 | 21064 | 21065 | 36 |
| 3024 | SAMD00009255.contig00005: 57581-59329 | 15116 | 15117 | 15118 | 15119 | 15120 | 37 |
| 5352 | SAMN08217911.contig00001: 500299-501714 | 26756 | 26757 | 26758 | 26759 | 26760 | 38 |
| 9064 | SAMN07659369.contig00012: 22375-24018 | 45316 | 45317 | 45318 | 45319 | 45320 | 39 |
| 12188 | SAMEA2056598.contig00009: 93335-94966 | 60936 | 60937 | 60938 | 60939 | 60940 | 40 |
| 11319 | SAMN02368459.contig00005: 42399-44366 | 56591 | 56592 | 56593 | 56594 | 56595 | 41 |
| 4421 | NAMP01000009.1: 271120-272949 | 22101 | 22102 | 22103 | 22104 | 22105 | 42 |
| 5387 | SAMEA3918403.contig00015: 32190-33752 | 26931 | 26932 | 26933 | 26934 | 26935 | 43 |
| 1465 | SAMN07155085.contig00017: 89966-91873 | 7321 | 7322 | 7323 | 7324 | 7325 | 44 |
| 1741 | SAMN06242082.contig00007: 156247-157722 | 8701 | 8702 | 8703 | 8704 | 8705 | 45 |
| 4031 | SAMEA3725544.contig00004: 79048-80670 | 20151 | 20152 | 20153 | 20154 | 20155 | 46 |
| 7810 | VYRD01000012.1: 141992-143485 | 39046 | 39047 | 39048 | 39049 | 39050 | 47 |
| 16 | SAMN07609731.contig00003: 28206-29882 | 76 | 77 | 78 | 79 | 80 | 48 |
| 4350 | SAMEA1919981.contig00001: 335197-336807 | 21746 | 21747 | 21748 | 21749 | 21750 | 49 |
| 3686 | SAMEA2040565.contig00003: 150-1589 | 18426 | 18427 | 18428 | 18429 | 18430 | 50 |
| 9395 | SAMEA2152096.contig00001: 82745-84373 | 46971 | 46972 | 46973 | 46974 | 46975 | 51 |
| 8012 | PNGL01000005.1: 218135-219571 | 40056 | 40057 | 40058 | 40059 | 40060 | 52 |
| 7146 | DS264285.1: 93661-95229 | 35726 | 35727 | 35728 | 35729 | 35730 | 53 |
| 1374 | SAMN08611390.contig00005: 113315-114871 | 6866 | 6867 | 6868 | 6869 | 6870 | 54 |
| 5286 | SAMEA3649730.contig00009: 34792-36417 | 26426 | 26427 | 26428 | 26429 | 26430 | 55 |
| 7380 | SAMEA3545329.contig00002: 40880-42541 | 36896 | 36897 | 36898 | 36899 | 36900 | 56 |
| 4056 | SAMEA69785668.contig00016: 50768-52345 | 20276 | 20277 | 20278 | 20279 | 20280 | 57 |
| 2101 | SAMEA1929523.contig00004: 163348-164733 | 10501 | 10502 | 10503 | 10504 | 10505 | 58 |
| 1122 | SAMEA2147867.contig00004: 1527-2939 | 5606 | 5607 | 5608 | 5609 | 5610 | 59 |
| 5743 | SAMN00691192.contig00011: 29686-31557 | 28711 | 28712 | 28713 | 28714 | 28715 | 60 |
| 8180 | VYVP01000025.1: 13412-15268 | 40896 | 40897 | 40898 | 40899 | 40900 | 61 |
| 9644 | CP053228.1: 5466927-5468669 | 48216 | 48217 | 48218 | 48219 | 48220 | 62 |
| 11016 | JAJBMY010000003.1: 112134-113783 | 55076 | 55077 | 55078 | 55079 | 55080 | 63 |
| 2190 | SAMEA4668412.contig00012: 26697-28496 | 10946 | 10947 | 10948 | 10949 | 10950 | 64 |
| 1511 | JAHLER010000006.1: 77201-78814 | 7551 | 7552 | 7553 | 7554 | 7555 | 65 |
| 11067 | JH992940.1: 172124-174154 | 55331 | 55332 | 55333 | 55334 | 55335 | 66 |
| 3449 | MCYX01000233.1: 17710-19281 | 17241 | 17242 | 17243 | 17244 | 17245 | 67 |
| 9646 | SAMN09980281.contig00004: 163373-164974 | 48226 | 48227 | 48228 | 48229 | 48230 | 68 |
| 5822 | SAMN06032688.contig00003: 203296-204942 | 29106 | 29107 | 29108 | 29109 | 29110 | 69 |
| 10869 | SAMN02363658.contig00001: 42204-43802 | 54341 | 54342 | 54343 | 54344 | 54345 | 70 |
| 11379 | JADNIM010000003.1: 38932-40590 | 56891 | 56892 | 56893 | 56894 | 56895 | 71 |
| 825 | QSVA01000008.1: 113741-115417 | 4121 | 4122 | 4123 | 4124 | 4125 | 72 |
| 4178 | SAMEA3572810.contig00007: 86149-87768 | 20886 | 20887 | 20888 | 20889 | 20890 | 73 |
| 11657 | SAMN08815326.contig00001: 429809-431185 | 58281 | 58282 | 58283 | 58284 | 58285 | 74 |
| 4341 | NUYS01000003.1: 6115-7488 | 21701 | 21702 | 21703 | 21704 | 21705 | 75 |
| 1494 | SAMEA3206487.contig00004: 184507-185949 | 7466 | 7467 | 7468 | 7469 | 7470 | 76 |
| 3021 | SAMEA3893659.contig00001: 85323-86831 | 15101 | 15102 | 15103 | 15104 | 15105 | 77 |
| 4674 | SAMEA2155293.contig00001: 623439-624914 | 23366 | 23367 | 23368 | 23369 | 23370 | 78 |
| 247 | SAMN07640820.contig00185: 192-2276 | 1231 | 1232 | 1233 | 1234 | 1235 | 79 |
| 3619 | CP066055.1: 203354-204886 | 18091 | 18092 | 18093 | 18094 | 18095 | 80 |
| 4477 | SAMEA103985801.contig00002: 117200-118897 | 22381 | 22382 | 22383 | 22384 | 22385 | 81 |
| 11492 | QVMC01000014.1: 27088-28758 | 57456 | 57457 | 57458 | 57459 | 57460 | 82 |
| 1433 | LZZO01000031.1: 239095-240519 | 7161 | 7162 | 7163 | 7164 | 7165 | 83 |
| 3415 | SAMN07974935.contig00001: 955948-957624 | 17071 | 17072 | 17073 | 17074 | 17075 | 84 |
| 8214 | JADNKD010000015.1: 58134-59807 | 41066 | 41067 | 41068 | 41069 | 41070 | 85 |
| 1390 | SAMD00010696.contig00004: 222324-224033 | 6946 | 6947 | 6948 | 6949 | 6950 | 86 |
| 6847 | SAMEA29984668.contig00007: 3876-5468 | 34231 | 34232 | 34233 | 34234 | 34235 | 87 |
| 6892 | SAMEA1034577.contig00028: 17682-19277 | 34456 | 34457 | 34458 | 34459 | 34460 | 88 |
| 2335 | SAMEA30012418.contig00001: 93358-95229 | 11671 | 11672 | 11673 | 11674 | 11675 | 89 |
| 11399 | JAGDJM010000001.1: 989306-990943 | 56991 | 56992 | 56993 | 56994 | 56995 | 90 |
| 7515 | SAMEA104076892.contig00004: 414361-416295 | 37571 | 37572 | 37573 | 37574 | 37575 | 91 |
| 2873 | SAMEA3512032.contig00006: 226692-228626 | 14361 | 14362 | 14363 | 14364 | 14365 | 92 |
| 8238 | JAEHJY010000005.1: 636416-637939 | 41186 | 41187 | 41188 | 41189 | 41190 | 93 |
| 9090 | SAMN09758972.contig00023: 19764-21332 | 45446 | 45447 | 45448 | 45449 | 45450 | 94 |
| 10874 | SAMN07658784.contig00014: 50878-52341 | 54366 | 54367 | 54368 | 54369 | 54370 | 95 |
| 8823 | LRFT01000008.1: 299384-301111 | 44111 | 44112 | 44113 | 44114 | 44115 | 96 |
| 2756 | SAMEA2273751.contig00021: 4220-6019 | 13776 | 13777 | 13778 | 13779 | 13780 | 97 |
| 3103 | SAMN09655750.contig00001: 23615-24967 | 15511 | 15512 | 15513 | 15514 | 15515 | 98 |
| 411 | SAMN09384874.contig00003: 69752-71404 | 2051 | 2052 | 2053 | 2054 | 2055 | 99 |
| 56 | JADNBE010000007.1: 26250-27977 | 276 | 277 | 278 | 279 | 280 | 100 |
| 5624 | SAMN07659792.contig00007: 108119-109519 | 28116 | 28117 | 28118 | 28119 | 28120 | 101 |
| 10493 | SAMN07135203.contig00014: 76689-78440 | 52461 | 52462 | 52463 | 52464 | 52465 | 102 |
| 4226 | SAMN09849028.contig00001: 144792-146207 | 21126 | 21127 | 21128 | 21129 | 21130 | 103 |
| 239 | SAMN09769763.contig00015: 28882-30276 | 1191 | 1192 | 1193 | 1194 | 1195 | 104 |
| 7584 | JAAQZA010000003.1: 291159-292913 | 37916 | 37917 | 37918 | 37919 | 37920 | 105 |
| 5383 | CP071739.1: 1336980-1338500 | 26911 | 26912 | 26913 | 26914 | 26915 | 106 |
| 3807 | JAJQFN010000527.1: 808-2259 | 19031 | 19032 | 19033 | 19034 | 19035 | 107 |
| 10421 | CP056148.1: 3947717-3949513 | 52101 | 52102 | 52103 | 52104 | 52105 | 108 |
| 4679 | CZAL01000004.1: 68838-70718 | 23391 | 23392 | 23393 | 23394 | 23395 | 109 |
| 10878 | SAMEA4470192.contig00005: 237352-239097 | 54386 | 54387 | 54388 | 54389 | 54390 | 110 |
| 7017 | CP045814.1: 2354008-2355576 | 35081 | 35082 | 35083 | 35084 | 35085 | 111 |
| 3786 | CP010106.1: 2351151-2352614 | 18926 | 18927 | 18928 | 18929 | 18930 | 112 |
| 2725 | FKZR01000004.1: 346065-347810 | 13621 | 13622 | 13623 | 13624 | 13625 | 113 |
| 29 | JAHOHS010000041.1: 18214-19956 | 141 | 142 | 143 | 144 | 145 | 114 |
| 790 | SAMN09671422.contig00009: 36535-38268 | 3946 | 3947 | 3948 | 3949 | 3950 | 115 |
| 763 | SAMN05444063.contig00002: 400236-401840 | 3811 | 3812 | 3813 | 3814 | 3815 | 116 |
| 2047 | SAMEA4550069.contig00003: 99226-100683 | 10231 | 10232 | 10233 | 10234 | 10235 | 117 |
| 9417 | BBDT01000003.1: 22618-24234 | 47081 | 47082 | 47083 | 47084 | 47085 | 118 |
| 3526 | JTES01000002.1: 318539-320254 | 17626 | 17627 | 17628 | 17629 | 17630 | 119 |
| 9701 | JAJBNY010000010.1: 80455-82032 | 48501 | 48502 | 48503 | 48504 | 48505 | 120 |
| 3469 | JAUE01000036.1: 18751-20418 | 17341 | 17342 | 17343 | 17344 | 17345 | 121 |
| 4295 | SAMN02693865.contig00009: 18041-19795 | 21471 | 21472 | 21473 | 21474 | 21475 | 122 |
| 3092 | SIYA01000011.1: 72929-74530 | 15456 | 15457 | 15458 | 15459 | 15460 | 123 |
| 4304 | SAMEA4427736.contig00005: 191907-193790 | 21516 | 21517 | 21518 | 21519 | 21520 | 124 |
| 2523 | SAMN02923848.contig00002: 177553-179199 | 12611 | 12612 | 12613 | 12614 | 12615 | 125 |
| 2521 | SAMN09384789.contig00001: 117383-119221 | 12601 | 12602 | 12603 | 12604 | 12605 | 126 |
| 2497 | SAMN07659527.contig00014: 98872-100659 | 12481 | 12482 | 12483 | 12484 | 12485 | 127 |
| 8 | SAMEA2266828.contig00014: 199-1962 | 36 | 37 | 38 | 39 | 40 | 128 |
| 9695 | JAFFRR010000020.1: 109299-110684 | 48471 | 48472 | 48473 | 48474 | 48475 | 129 |
| 7033 | SAMN02934513.contig00004: 266274-268163 | 35161 | 35162 | 35163 | 35164 | 35165 | 130 |
| 4195 | SAMN06187708.contig00002: 307109-308617 | 20971 | 20972 | 20973 | 20974 | 20975 | 131 |
| 937 | SAMN07661511.contig00025: 11621-13285 | 4681 | 4682 | 4683 | 4684 | 4685 | 132 |
| 130 | CP017112.1: 2244961-2246535 | 646 | 647 | 648 | 649 | 650 | 133 |
| 2565 | JAFHCM010000008.1: 66093-67844 | 12821 | 12822 | 12823 | 12824 | 12825 | 134 |
| 9949 | QDER01000003.1: 3046-4452 | 49741 | 49742 | 49743 | 49744 | 49745 | 135 |
| 10362 | SAMN07534973.contig00001: 114744-116135 | 51806 | 51807 | 51808 | 51809 | 51810 | 136 |
| 7769 | SAMEA3473579.contig00002: 659699-661366 | 38841 | 38842 | 38843 | 38844 | 38845 | 137 |
| 396 | CP071326.1: 205471-207492 | 1976 | 1977 | 1978 | 1979 | 1980 | 138 |
| 8354 | SAMN06299513.contig00003: 467425-469221 | 41766 | 41767 | 41768 | 41769 | 41770 | 139 |
| 11676 | SAMN07609274.contig00016: 21590-23158 | 58376 | 58377 | 58378 | 58379 | 58380 | 140 |
| 10895 | SAMEA3866237.contig00006: 64775-66142 | 54471 | 54472 | 54473 | 54474 | 54475 | 141 |
| 12706 | SAMD00002831.contig00017: 75582-77144 | 63526 | 63527 | 63528 | 63529 | 63530 | 142 |
| 12097 | SAMN05710316.contig00033: 50360-52027 | 60481 | 60482 | 60483 | 60484 | 60485 | 143 |
| 5955 | SAMN02356610.contig00006: 156412-157920 | 29771 | 29772 | 29773 | 29774 | 29775 | 144 |
| 49 | SAMN04376559.contig00001: 34912-36546 | 241 | 242 | 243 | 244 | 245 | 145 |
| 2671 | SAMEA1530134.contig00001: 83143-84810 | 13351 | 13352 | 13353 | 13354 | 13355 | 146 |
| 9726 | SAMEA2247577.contig00011: 149643-151286 | 48626 | 48627 | 48628 | 48629 | 48630 | 147 |
| 4256 | SAMN04123844.contig00002: 201133-202839 | 21276 | 21277 | 21278 | 21279 | 21280 | 148 |
| 11735 | NFHM01000012.1: 72850-74532 | 58671 | 58672 | 58673 | 58674 | 58675 | 149 |
| 5426 | NFHY01000001.1: 135829-137451 | 27126 | 27127 | 27128 | 27129 | 27130 | 150 |
| 1159 | CP026362.1: 1564096-1565772 | 5791 | 5792 | 5793 | 5794 | 5795 | 151 |
| 7398 | SAMEA2710612.contig00004: 131882-133273 | 36986 | 36987 | 36988 | 36989 | 36990 | 152 |
| 5984 | SAMEA1566194.contig00005: 10675-12114 | 29916 | 29917 | 29918 | 29919 | 29920 | 153 |
| 3397 | JADMOI010000001.1: 158941-160779 | 16981 | 16982 | 16983 | 16984 | 16985 | 154 |
| 7963 | SAMEA3357052.contig00010: 63403-65097 | 39811 | 39812 | 39813 | 39814 | 39815 | 155 |
| 1310 | PSNF01000030.1: 58968-60821 | 6546 | 6547 | 6548 | 6549 | 6550 | 156 |
| 7360 | SAMN05294119.contig00004: 257440-259095 | 36796 | 36797 | 36798 | 36799 | 36800 | 157 |
| 577 | QYTJ01000020.1: 90589-92004 | 2881 | 2882 | 2883 | 2884 | 2885 | 158 |
| 5782 | SAMEA2205381.contig00006: 31023-32804 | 28906 | 28907 | 28908 | 28909 | 28910 | 159 |

For each cluster, the corresponding attB sequences of each LSR protein were aligned to infer specificity of each LSR cluster's targeting sites (higher attB sequence identity indicates that the landing sites are likely to be more specific). Based on the inferred specificity score, the 159 LSR clusters were grouped into one of two categories: “putative multi-targeting LSRs” or “putative specific LSRs”. To prepare an attD sequence of each LSR for the screening, the center dinucleotides of the original attP sequence were modified to ensure 1) the dinucleotides are in not in palindromic pattern (AT, TA, CG, or GC); and 2) each attD sequence had a minimum number of mismatches against the human reference genome (hg38).

### Synthesis and Cloning

AttD-LSR fragments were synthesized by Twist Biosciences with homology arms for gibson assembly. The fragments were validated by Oxford Nanopore Long-Read sequencing and pooled into specific and multi-targeting LSR pools based on attB-consensus within the cluster. These fragments were inserted into a backbone downstream of a CMV promoter, with a 3′ Nuclear Localization Sequence (NLS) for nuclear targeting of proteins to target the genome i/? cellulo, and with a Puromycin resistance gene, using NEBuilder® HiFi DNA Assembly Master Mix (M5520A VIAL). Resulting plasmids were then transformed into NEB® Stable Competent E. coli (High Efficiency) (C3040IVIAL) to generate two libraries (one including the specific LSR pool and the other including the multi-targeting LSR pool). Both libraries had a coverage of 56,470× calculated via colony counts of serial dilution onto agar-carbenicillin plates.

AttA Recombination plasmids were cloned from oligo pools generated by Twist Biosciences using NEBridge® Golden Gate Enzyme Mix (BsmBI-v2) (M2617AAVIAL). The library coverage was determined to be 1,294× as described above. The libraries were sequenced via Oxford Nanopore Long read sequencing to validate unbiased cloning and representation of all LSRs within the pool.

### Plasmid Recombination Assay

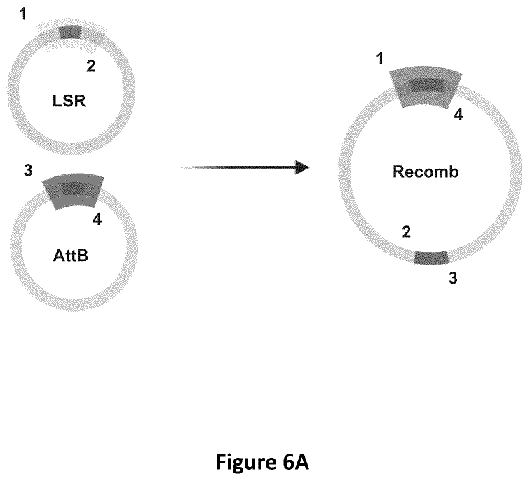

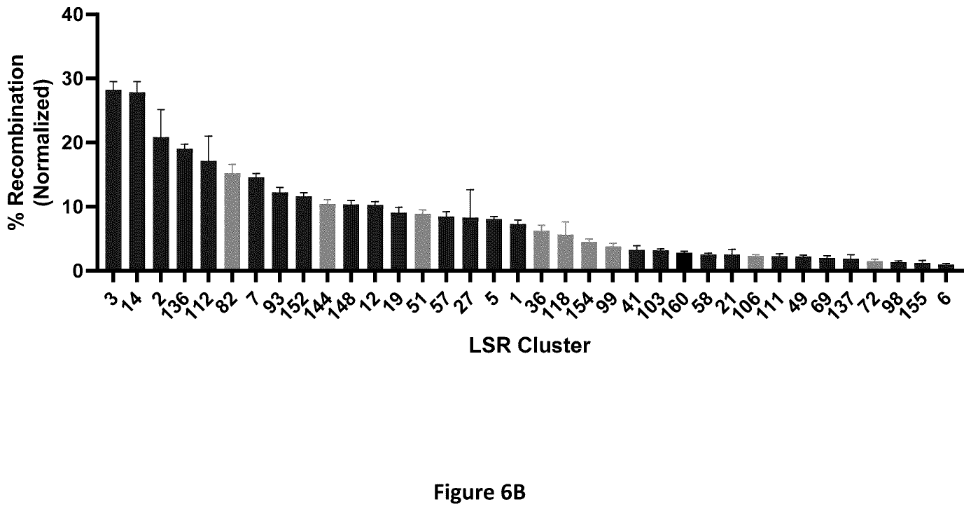

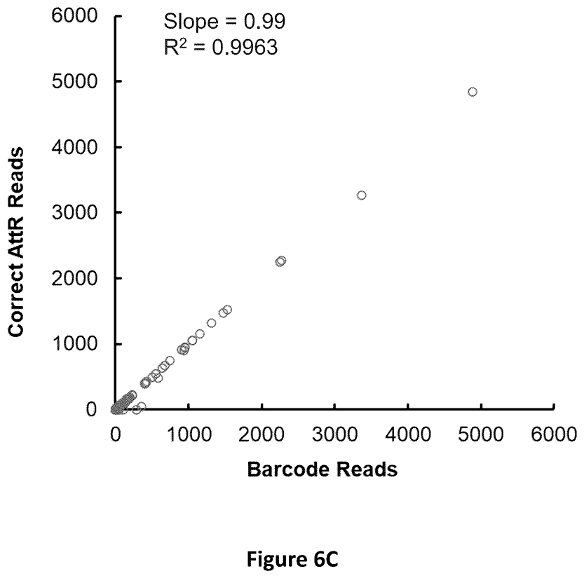

The same protocol as described above for the individual LSR screening was also used with the pooled LSR libraries, but an Illumina sequencing NGS readout was used to determine which barcodes recombined (illustrated in FIG. 6A), based on counts within the amplicons. These were normalized to the starting % of reads of each LSR and attA plasmid in the library and compared to a Bxb1 positive control.

### Genomic Integration Assay

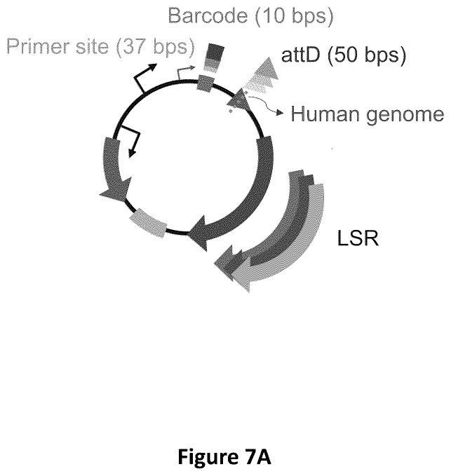

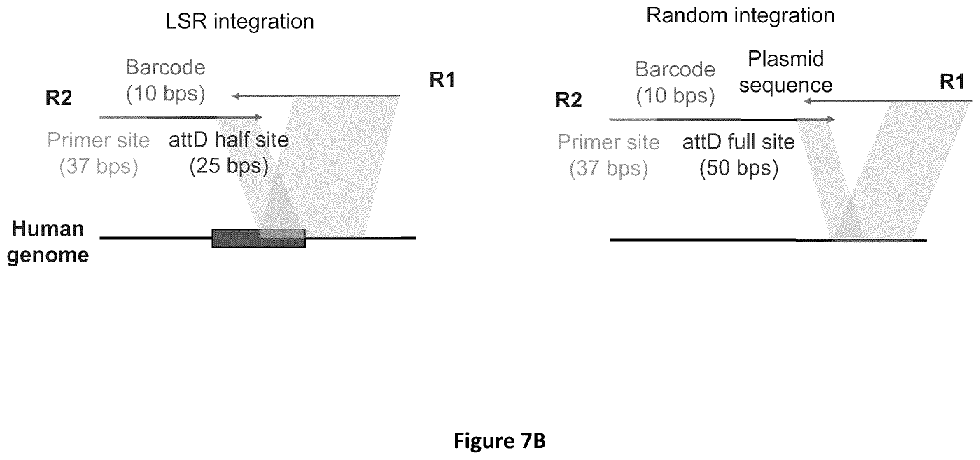

HEK-293T cells were transfected with a multi-targeting or specific LSR library as described above. Cells were selected with 1 μg/mL of Puromycin to enrich cells that had plasmid integration. Selection began at day 2 and continued until day 18 post-transfection. Genomic DNA was isolated from the Puromycin positive cells and genomic integration was determined via sequencing of barcodes (illustrated in FIGS. 7A and 7B).

### ILL-seq

For Illumina amplicon sequencing, two rounds of amplification were performed: round 1 PCR was performed in a 12 μL reaction volume, comprising 6 μL of NEBNext® Ultra™ II Q5® Master Mix (New England Biolabs), 0.25 μM forward and reverse primer, and 20 ng of gDNA template. PCR conditions were as follows: 30 seconds at 98° C. for initial denaturation, followed by 20 cycles of 10 seconds at 98ºC for denaturation, 15 seconds at 60ºC for annealing, 30 seconds at 72ºC for extension, and 5 minutes at 72ºC for the final extension. Round 2 PCR was performed in a 12 μl reaction volume, consisting of 6 μL of NEBNext® Ultra™ II Q5® Master Mix (New England Biolabs), 1 μM forward and reverse primers, and 4 μl of PCR Round 1 product. PCR conditions were as follows: 30 seconds at 98° C. for initial denaturation, followed by 14 cycles of 10 seconds at 98ºC for denaturation, 15 seconds at 60ºC for annealing, 30 seconds at 72ºC for extension, and 5 minutes at 72° C. for the final extension. The PCR reactions that were to be combined into a sequencing library were pooled and purified using AMPure XP beads (Beckman Coulter) as per the manufacturer's protocol. Purified products were size selected in the 300 to 1200 base pair range using a BluePippin (Sage Science) and re-purified with AMPure XP beads (Beckman Coulter). 8-10 pmol of sequencing library were analyzed via MiSeq Reagent Kit v3 with 10-15% PhiX Control v3 (Illumina) to obtain 2×300 cycle reads. Source code and data analytical methods are as described in Maeder et al., 2019 Nature Medicine 25:229-233.

### UDiTaS

For measuring genomic integration, sequencing libraries were prepared using the UDiTaS protocol according to the publication Giannoukos et al., 2018 with some minor modifications. Briefly, 50 ng gDNA was used as input into the tagmentation reaction; 4 μL nuclease free water, 2 μL 1 mg/mL transposome (Tn5 complexed with custom barcoded oligo), 4 μL 5× TAPS-DMF buffer and 10 μL DNA (10 ng/μL), which was incubated at 55° C. for 7 minutes and placed on ice. To inactivate the transposase, 1 μL of Proteinase K (NEB, P8107S) was added to each tagmented reaction, mixed well and placed on the thermal cycler (37° C. for 1 hour, 95° C. 10 minutes and 4° C. hold) followed by AMPure XP (1×) clean up according to the manufacturer's protocol. Round 1 PCR volume was increased to 50 μL final volume: 25 μL 2× Platinum SuperFi Master mix (12358-010, ThermoFisher Scientific), 3 μL 0.5 M Tetramethylammonium chloride (TMAC; T3411, Sigma-Aldrich), 1.25 μL 10 μM P5 primer, 0.375 μL 100 μM assay specific primer and 20.5 μL tagmented DNA. Round 1 PCR conditions were as follows: 98° C. for 2 minutes followed by 15 cycles of 98° C. for 10 seconds, 65° C. for 10 seconds, and 72ºC for 90 seconds and a final extension of 72ºC for 5 minutes. Round 1 PCR products were cleaned up with Ampure XP (0.9×) according to the manufacturer's protocol and eluted in 15 μL nuclease free water directly into the round 2 PCR mix: 25 μL 2× Platinum SuperFi Master mix (12358-010, ThermoFisher Scientific), 2.5 μL 10 μM P5 primer, 7.5 μL 10 μM UDiTaS Round 2 P7_bc_SBS12 primer. Round 2 PCR conditions were as follows: 98° C. for 2 minutes followed by 15 cycles of 98° C. for 10 seconds, 65° C. for 10 seconds, and 72° ° C. for 90 seconds and a final extension of 72ºC for 5 minutes. Round 2 products were cleaned up with Ampure XP (0.9×) according to the manufacturer's protocol and run on the Agilent Tapestation 4200 using the D5000 tapes for quantification and sizing of the products to calculate nM for pooling. AMPure XP clean-up was increased to 1.2× reaction volume after pooling and to 1.5× reaction volume after size selection on BluePippin (400-850 bp). Library quantification was performed using Qubit dsDNA HS assay to determine concentration (ng/μL) (Q32851: ThermoFisher Scientific) and Agilent Bioanalyzer High Sensitivity DNA Kit (5067-4626: Agilent) for size (bp) in order to calculate the nM. The sequencing library (9 pM) was loaded into an Illumina MiSeq Reagent kit v3 containing 4.2% 20 pM PhiX Control v3 (Illumina #FC-110-3001) to obtain 2×300 cycle reads and index reads (8 and 18 bp).

### Analysis

For Illumina sequencing analysis of plasmid recombination, the reads from each LSR plasmid were identified and classified by searching the concatenated sequence of corresponding 10-bp barcode plus the first 20-bp of attD (>=90% sequence identity). Then, the attR sequence of each LSR was generated by concatenating the attD left half-site and the attA right half-site. The number of reads that contained the attR sequence (>=90% sequence identity) indicated the expected recombined plasmid and was counted for each LSR group.

For UDiTaS sequencing analysis of human genome integration, sequencing read pairs generated using the UDiTas protocol were first aligned to a representative LSR plasmid sequence (LSR plasmid for cluster 1), and then aligned to human reference genome (hg38) using Bowtie2 aligner (Langmead and Salzberg, 2002). The integrations to human genome were detected by searching the read-pairs, with R1 reads being aligned to human reference genome and R2 reads being partially aligned to the LSR plasmid sequence and human reference genome. The 10-bp barcode sequences in the R2 reads were used to differentiate LSRs. The exact positions of cut sites in the plasmid sequence and the integration sites in the human genome were determined based on the coordinates of R2 read alignments to the human genome. Finally, the reads with the same Unique Molecular Identifiers (UMI) were collapsed to remove duplicated reads due to PCR amplification. The results from these analyses are summarized in Table 4.

TABLE 4
LSR Functional Annotations

| | | | dis- | | |
|---|---|---|---|---|---|
| | | | tance_ | | |
| | | | to_ | | |
| | | | expec- | umi_ | |
| lsr_ | | umi_ | ted_ | frac- | functional_ |
| cluster | landing_site | count | cut | tion | annotation |
| PRO426 | chr1: | 368 | 6 | 21.67 | exon 2 of the |
| | 15835976 | | | | lncRNA |
| | | | | | AL450998.2 |
| | | | | | ENST00000317122) |
| PRO426 | chr4: | 197 | 0 | 11.6 | intron 3 of the gene |
| | 36049472 | | | | ARAP2 |
| | | | | | (ENST00000503225) |
| | | | | | and is 2533-bp from |
| | | | | | exon 4 |
| PRO426 | chr3: | 124 | 0 | 7.3 | intron 11 of the gene |
| | 4717822 | | | | ITPR1 |
| | | | | | (ENST00000648016) |
| | | | | | and is 422-bp |
| | | | | | from exon |
| | | | | | 11 |
| PRO426 | chr7: | 86 | 0 | 5.06 | intergenic region |
| | 135538107 | | | | and is 19810-bp |
| | | | | | from the gene |
| | | | | | NUP205 |
| | | | | | (ENST00000285968) |
| c11 | chr4: | 30 | 5 | 41.67 | intron 1 of |
| | 136332691 | | | | the lncRNA |
| | | | | | AC018680.1 |
| | | | | | (ENST00000500324) |
| | | | | | and is 62459-bp from |
| | | | | | exon 2 |
| c11 | chr7: | 26 | 5 | 36.11 | intergenic region |
| | 17948577 | | | | and is 5655-bp |
| | | | | | from the TEC |
| | | | | | gene AC080080.1 |
| | | | | | (ENST00000625121) |
| c11 | chr7: | 12 | 5 | 16.67 | intron 2 of |
| | 25395275 | | | | the lncRNA |
| | | | | | AC005100.1 |
| | | | | | (ENST00000668357) |
| | | | | | and is 2211-bp from |
| | | | | | exon 3 |
| c6 | chrX: | 44 | 12 | 97.78 | intron 1 of the gene |
| | 86717740 | | | | DACH2 |
| | | | | | (ENST00000484479) |
| | | | | | and is 3019-bp from |
| | | | | | exon 1 |
| c16 | chr4: | 74 | 3 | 54.01 | intergenic |
| | 39400428 | | | | region and is |
| | | | | | 1180-bp from the |
| | | | | | snRNA RNU6 |
| | | | | | (ENST00000410660) |
| c16 | chr8: | 52 | 1 | 37.96 | intergenic |
| | 48523362 | | | | region and is |
| | | | | | 5205-bp from the |
| | | | | | lncRNA AC026904.1 |
| | | | | | (ENST00000665034) |
| c16 | chrX: | 11 | 3 | 8.03 | intergenic |
| | 149757546 | | | | region and is |
| | | | | | 251-bp from the |
| | | | | | pseudogene |
| | | | | | AC244098.2 |
| | | | | | (ENST00000422068) |
| c18 | chr13: | 31 | 5 | 96.88 | intron 2 of the gene |
| | 66866243 | | | | PCDH9 |
| | | | | | (ENST00000544246) |
| | | | | | and is 234831-bp |
| | | | | | from |
| | | | | | exon 2 |
| c19 | chr7: | 64 | 2 | 25 | intergenic |
| | 25862013 | | | | region and is |
| | | | | | 30104-bp from the |
| | | | | | lncRNA AC018706.1 |
| | | | | | (ENST00000666265) |
| c19 | chr8: | 28 | 6 | 10.94 | intron 1 of the gene |
| | 106661826 | | | | OXR1 |
| | | | | | (ENST00000497705) |
| | | | | | and is 17383-bp from |
| | | | | | exon 2 |
| c19 | chr13: | 25 | 0 | 9.77 | intron 1 of |
| | 62721865 | | | | the lncRNA |
| | | | | | LINC00448 |
| | | | | | (ENST00000448411) |
| | | | | | and is 10228-bp from |
| | | | | | exon 2 |
| c19 | chr18: | 25 | 0 | 9.77 | intergenic region and |
| | 78725856 | | | | there are no genes |
| | | | | | within 50 kB |
| c19 | chr1: | 22 | 0 | 8.59 | intergenic |
| | 180592222 | | | | region and is |
| | | | | | 25702-bp from the |
| | | | | | lncRNA OVAAL |
| | | | | | (ENST00000648175) |
| c19 | chr2: | 18 | 2 | 7.03 | intron 12 of the |
| | 88796321 | | | | pseudogene |
| | | | | | ANKRD36BP2 |
| | | | | | (ENST00000393515) |
| | | | | | and is 3826-bp from |
| | | | | | exon 12 |
| c19 | chr3: | 15 | 1 | 5.86 | intergenic region |
| | 160642025 | | | | and is 35135-bp |
| | | | | | from the gene |
| | | | | | ARL 14 |
| | | | | | (ENST00000320767) |
| c19 | chr4: | 13 | 0 | 5.08 | intron 1 of the gene |
| | 127965441 | | | | MFSD8 |
| | | | | | (ENST00000641447) |
| | | | | | and is 218-bp |
| | | | | | from exon 1 |
| c27 | chr8: | 18 | 1 | 58.06 | intron 2 of |
| | 59614252 | | | | the lncRNA |
| | | | | | AC087664.2 |
| | | | | | (ENST00000653946) |
| | | | | | and is 1362-bp from |
| | | | | | exon 2 |
| c27 | chrX: | 13 | 8 | 41.94 | intergenic |
| | 103553615 | | | | region and is |
| | | | | | 10302-bp from the |
| | | | | | lncRNA AL021308.1 |
| | | | | | (ENST00000655887) |
| c75 | chr17: | 44 | 2 | 95.65 | intron 10 of the gene |
| | 41955533 | | | | TTC25 |
| | | | | | (ENST00000377540) |
| | | | | | and is 215-bp |
| | | | | | from exon 10 |
| c76 | chr13: | 55 | 11 | 100 | intergenic |
| | 63797320 | | | | region and is |
| | | | | | 13416-bp from the |
| | | | | | snRNA RNU6 |
| | | | | | (ENST00000365608) |
| c77 | chr5: | 111 | 2 | 26.12 | intron 5 of the gene |
| | 26905860 | | | | CDH9 |
| | | | | | (ENST00000231021) |
| | | | | | and is 98-bp |
| | | | | | from exon 6 |
| c77 | chr1: | 71 | 1 | 16.71 | intergenic |
| | 227389055 | | | | region and is |
| | | | | | 4499-bp from the |
| | | | | | lncRNA LINC01641 |
| | | | | | (ENST00000660249) |
| c77 | chr2: | 28 | 0 | 6.59 | intron 1 of the gene |
| | 213638073 | | | | SPAG16 |
| | | | | | (ENST00000451561) |
| | | | | | and is 147982-bp |
| | | | | | from exon 1 |
| c77 | chr9: | 27 | 0 | 6.35 | intron 5 of the |
| | 82518940 | | | | pseudogene |
| | | | | | AL162726.3 |
| | | | | | (ENST00000586399) |
| | | | | | and is 1733-bp from |
| | | | | | exon 6 |
| c77 | chr1: | 22 | 4 | 5.18 | intron 1 of the gene |
| | 109396337 | | | | SORT1 |
| | | | | | (ENST00000256637) |
| | | | | | and is 1249-bp from |
| | | | | | exon 2 |
| c77 | chr17: | 22 | 0 | 5.18 | intron 1 of the gene |
| | 48206495 | | | | SKAP1 |
| | | | | | (ENST00000581400) |
| | | | | | and is 16994-bp from |
| | | | | | exon 1 |
| c84 | chr10: | 26 | 1 | 15.66 | intron 1 of the gene |
| | 15858956 | | | | MINDY3 |
| | | | | | (ENST00000277632) |
| | | | | | and is 1249-bp from |
| | | | | | exon 2 |
| c84 | chr1: | 22 | 0 | 13.25 | intron 1 of the gene |
| | 88687296 | | | | PKN2 |
| | | | | | (ENST00000316005) |
| | | | | | and is 2667-bp from |
| | | | | | exon 1 |
| c84 | chr10: | 19 | 1 | 11.45 | intergenic |
| | 132014516 | | | | region and is |
| | | | | | 14670-bp from |
| | | | | | the TEC |
| | | | | | gene AL162274.3 |
| | | | | | (ENST00000623138) |
| c84 | chr7: | 18 | 0 | 10.84 | intron 2 of the gene |
| | 114618539 | | | | FOXP2 |
| | | | | | (ENST00000360232) |
| | | | | | and is 10000-bp from |
| | | | | | exon 3 |
| c84 | chr2: | 16 | 1 | 9.64 | intron 1 of |
| | 212915149 | | | | the lncRNA |
| | | | | | AC093865.1 |
| | | | | | (ENST00000415387) |
| | | | | | and is 11705-bp |
| | | | | | from exon 1 |
| c84 | chr3: | 15 | 0 | 9.04 | intron 1 of the |
| | 168297568 | | | | pseudogene |
| | | | | | EGFEM1P |
| | | | | | (ENST00000502332) |
| | | | | | and is 11736-bp from |
| | | | | | exon 2 |
| c84 | chr13: | 13 | 0 | 7.83 | intron 1 of the gene |
| | 93280879 | | | | GPC6 |
| | | | | | (ENST00000377047) |
| | | | | | and is 53262-bp from |
| | | | | | exon 1 |
| c84 | chr12: | 11 | 0 | 6.63 | intron 1 of the gene |
| | 63918832 | | | | SRGAP1 |
| | | | | | (ENST00000355086) |
| | | | | | and is 65114-bp from |
| | | | | | exon 2 |
| c84 | chr3: | 11 | 0 | 6.63 | intergenic |
| | 114123046 | | | | region and is |
| | | | | | 5606-bp from the |
| | | | | | gene DRD3 |
| | | | | | (ENST00000460779) |
| c85 | chr4: | 138 | 15 | 99.28 | intron 1 of the gene |
| | 90847539 | | | | CCSER1 |
| | | | | | (ENST00000515693) |
| | | | | | and is 31693-bp from |
| | | | | | exon 1 |
| c93 | chr13: | 66 | 2 | 21.36 | intron 6 of the gene |
| | 71533910 | | | | DACH1 |
| | | | | | (ENST00000613252) |
| | | | | | and is 23113-bp from |
| | | | | | exon 7 |
| c93 | chr15: | 48 | 3 | 15.53 | intergenic |
| | 95944076 | | | | region and is |
| | | | | | 46506-bp from the |
| | | | | | lncRNA AC012409.2 |
| | | | | | (ENST00000619812) |
| c93 | chr7: | 38 | 0 | 12.3 | intron 4 of the gene |
| | 26750804 | | | | SKAP2 |
| | | | | | (ENST00000345317) |
| | | | | | and is 10839-bp from |
| | | | | | exon 4 |
| c93 | chr1: | 34 | 1 | 11 | intergenic |
| | 33458319 | | | | region and is |
| | | | | | 7930-bp from the |
| | | | | | pseudogene TLR12P |
| | | | | | (ENST00000413515) |
| c93 | chr21: | 18 | 2 | 5.83 | intron 1 of |
| | 16322103 | | | | the lncRNA |
| | | | | | MIR99AHG |
| | | | | | (ENST00000654997) |
| | | | | | and is 127469-bp |
| | | | | | from exon 1 |
| c93 | chr9: | 16 | 2 | 5.18 | intergenic |
| | 122906773 | | | | region and is |
| | | | | | 1283-bp from the |
| | | | | | gene ZBTB6 |
| | | | | | (ENST00000373659) |
| c93 | chr14: | 16 | 10 | 5.18 | intron 1 of |
| | 57077739 | | | | the lncRNA |
| | | | | | AL391152.1 |
| | | | | | (ENST00000551408) |
| | | | | | and is 10573-bp from |
| | | | | | exon 1 |
| c103 | chr5: | 54 | 0 | 61.36 | intron 1 of the |
| | 34187488 | | | | pseudogene |
| | | | | | AC138409.2 |
| | | | | | (ENST00000514048) |
| | | | | | and is 1983-bp from |
| | | | | | exon 2 |
| c103 | chr5: | 24 | 0 | 27.27 | intron 1 of the gene |
| | 66893032 | | | | MAST4 |
| | | | | | (ENST00000403666) |
| | | | | | and is 6918-bp from |
| | | | | | exon 2 |
| c104 | chr2: | 19 | 1 | 44.19 | intergenic region and |
| | 175681273 | | | | there are no genes |
| | | | | | within 50 kB |
| c104 | chr9: | 12 | 8 | 27.91 | intron 5 of the gene |
| | 9572566 | | | | PTPRD |
| | | | | | (ENST00000381196) |
| | | | | | and is 2165-bp from |
| | | | | | exon 6 |
| c104 | chr9: | 4 | 8 | 9.3 | intron 5 of the gene |
| | 9572573 | | | | PTPRD |
| | | | | | (ENST00000381196) |
| | | | | | and is 2158-bp from |
| | | | | | exon 6 |
| c104 | chr2: | 3 | 13 | 6.98 | intron 1 of the gene |
| | 212271439 | | | | ERBB4 |
| | | | | | (ENST00000260943) |
| | | | | | and is 146535-bp |
| | | | | | from exon 1 |
| c111 | chr10: | 41 | 5 | 56.16 | intron 1 of the gene |
| | 103183251 | | | | NT5C2 |
| | | | | | (ENST00000343289) |
| | | | | | and is 8268-bp from |
| | | | | | exon 1 |
| c111 | chr21: | 28 | 4 | 38.36 | intron 2 of the gene |
| | 33462603 | | | | IFNGR2 |
| | | | | | (ENST00000421802) |
| | | | | | and is 16194-bp from |
| | | | | | exon 3 |
| c111 | chr12: | 4 | 4 | 5.48 | intron 3 of the gene |
| | 4564682 | | | | DYRK4 |
| | | | | | (ENST00000539309) |
| | | | | | and is 183-bp from |
| | | | | | exon 3 |
| c112 | chrX: | 79 | 1 | 36.07 | intron 7 of the gene |
| | 23936860 | | | | CXorf58 |
| | | | | | (ENST00000379211) |
| | | | | | and is 1434-bp from |
| | | | | | exon 7 |
| c112 | chrX: | 33 | 2 | 15.07 | intron 7 of the gene |
| | 23936864 | | | | CXorf58 |
| | | | | | (ENST00000379211) |
| | | | | | and is 1438-bp from |
| | | | | | exon 7 |
| c112 | chr22: | 26 | 0 | 11.87 | intergenic region and |
| | 27624873 | | | | there are no genes |
| | | | | | within 50 kB |
| c112 | chr5: | 22 | 4 | 10.05 | intron 3 of the gene |
| | 93898542 | | | | FAM172A |
| | | | | | (ENST00000509739) |
| | | | | | and is 16853-bp from |
| | | | | | exon 3 |
| c112 | chr3: | 16 | 3 | 7.31 | intron 1 of the gene |
| | 168083868 | | | | GOLIM4 |
| | | | | | (ENST00000309027) |
| | | | | | and is 11230-bp from |
| | | | | | exon 2 |
| c136 | chr5: | 46 | 2 | 30.26 | intron 1 of |
| | 141210696 | | | | the lncRNA |
| | | | | | AC244517.11 |
| | | | | | (ENST00000624192) |
| | | | | | and is 30976-bp from |
| | | | | | exon 2 |
| c136 | chr8: | 42 | 2 | 27.63 | intron 1 of the gene |
| | 78582474 | | | | PKIA |
| | | | | | (ENST00000352966) |
| | | | | | and is 15883-bp from |
| | | | | | exon 2 |
| c136 | chr9: | 16 | 5 | 10.53 | intron 1 of the gene |
| | 1996360 | | | | SMARCA2 |
| | | | | | (ENST00000637383) |
| | | | | | and is 15973-bp from |
| | | | | | exon 1 |
| c136 | chr4: | 15 | 0 | 9.87 | intergenic |
| | 113870011 | | | | region and is |
| | | | | | 29775-bp from the |
| | | | | | pseudogene |
| | | | | | AC111193.1 |
| | | | | | (ENST00000504097) |
| c140 | chr2: | 77 | 15 | 53.85 | intron 8 of the gene |
| | 26938383 | | | | DPYSL5 |
| | | | | | (ENST00000288699) |
| | | | | | and is 1647-bp from |
| | | | | | exon 9 |
| c140 | chr7: | 28 | 2 | 19.58 | intergenic |
| | 27181341 | | | | region and is |
| | | | | | 169-bp from the gene |
| | | | | | HOXA11 |
| | | | | | (ENST00000517402) |
| c140 | chr11: | 9 | 0 | 6.29 | exon 1 of the |
| | 65422844 | | | | lncRNA NEAT1 |
| | | | | | (ENST00000499732) |
| c140 | chr14: | 8 | 0 | 5.59 | intergenic |
| | 62513194 | | | | region and is |
| | | | | | 1678-bp from the |
| | | | | | pseudogene |
| | | | | | AL389895.1 |
| | | | | | (ENST00000554127) |

### Results

Representative LSRs from each cluster described above (Table 2) were assayed in a pooled plasmid recombination assay (FIG. 6A). The LSRs were assayed in two separate pools, one pool corresponding to putative specific LSR clusters and the other to putative multi-targeting LSR clusters based on attB-consensus within the cluster. Results are shown in FIG. 6B. In FIG. 6B, LSRs from putative specific LSR clusters are shown in blue (clusters 3, 14, 2, 136, 112, 7, 93, 152, 148, 12, 19, 57, 27, 5, 1, 41, 103, 58, 21, 111, 49, 69, 137, 98, 155 and 6) and LSRs from putative multi-targeting LSR clusters are shown in red (clusters 82, 144, 51, 36, 118, 154, 99, 106, and 72). Positive control Bxb1 is shown as 160 in black. As depicted, many LSRs demonstrated efficient recombination. Representative LSRs from some clusters (e.g., clusters 3 and 14) demonstrated recombination levels that are 10-fold higher than Bxb1 control recombinase (FIG. 6B). Additionally, barcode reads and correct attR reads were highly correlated, thus confirming the orthogonality of the LSR clusters and accuracy of the target site prediction (FIG. 6C).

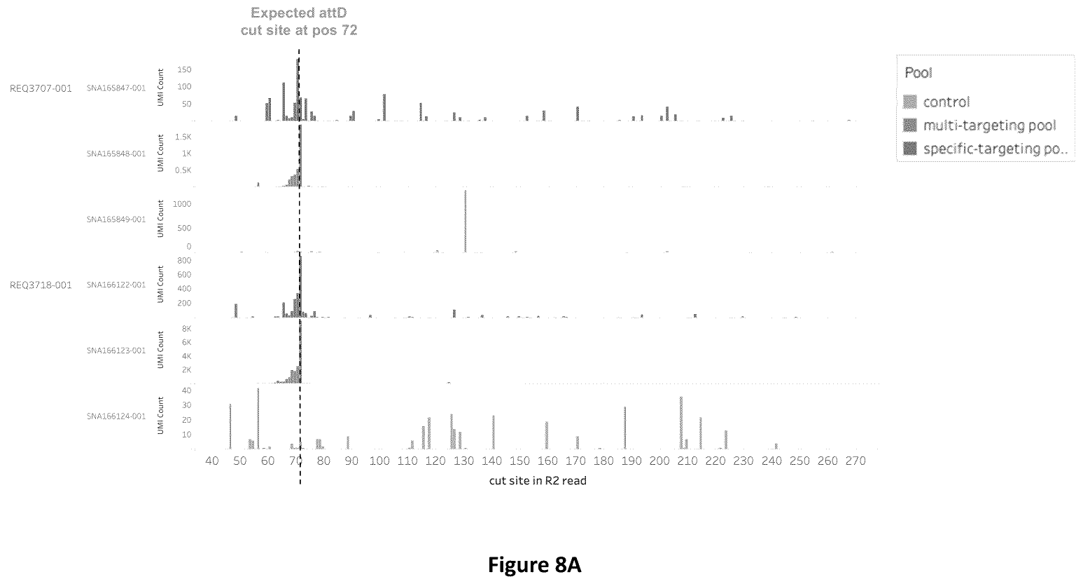

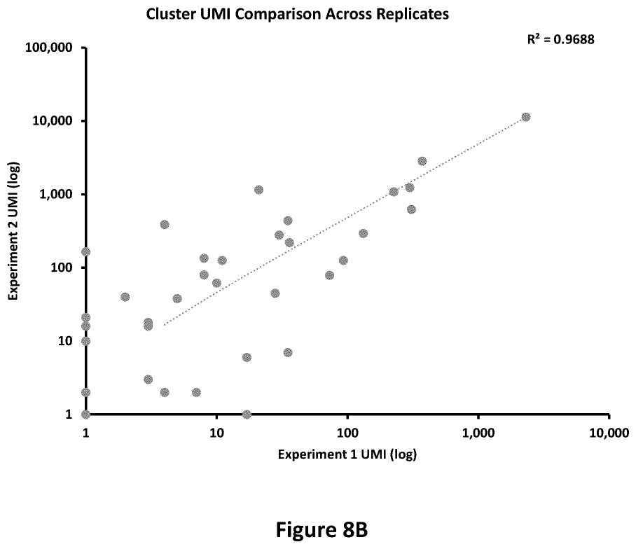

Representative LSRs from each cluster described above (Table 2) were also assayed in a pooled genomic integration assay (FIGS. 7A and 7B). As seen in FIG. 8A, the majority of the unique molecular identifiers (UMI) counts are observed at position 72 of next generation sequence (NGS) reads across two replicate experiments (FIG. 8A). This is consistent with LSR-mediated recombination at the central dinucleotide region of the attD sequence as a result of targeted integration rather than random plasmid integration. These results were observed for both the putative specific LSR cluster pool, and the putative multi-targeting LSR cluster pool, while the control samples lacking an LSR and attD site had no detectable targeted integration at position 72. Only reads with the expected cut site were analyzed. The integration events, as measured by UMI, were strongly correlated across the two replicate experiments (R²=0.9688, FIG. 8B).

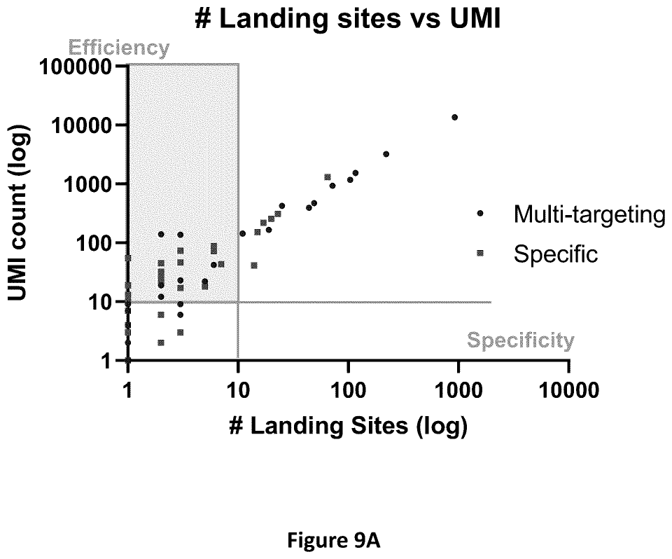

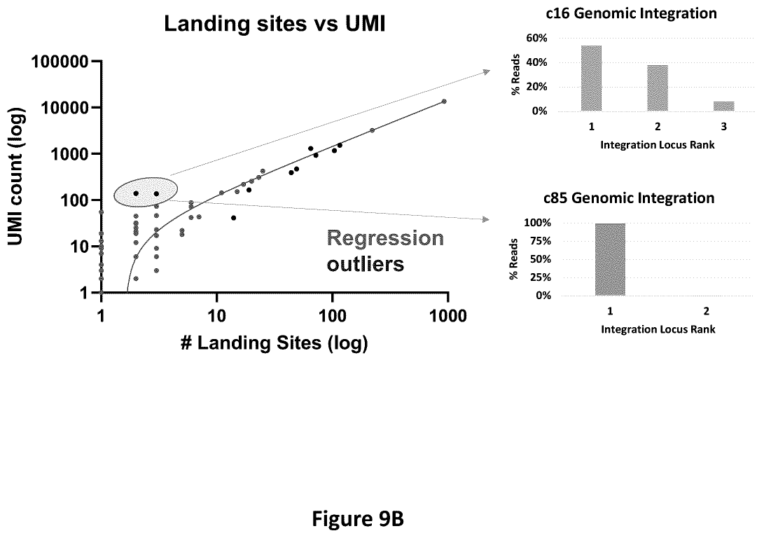

Further results from the pooled genomic integration assay are shown in FIGS. 9A and 9B, which depicts UMI count (as a measure of recombination activity) and number of landing sites in the human genome (as a measure of specificity) for each LSR tested. As depicted, many LSRs show integration into the human genome. Particularly promising LSRs for single effector gene therapy are highlighted in the top, left shaded quadrant. These LSRs have high UMI counts (indication of recombination activity) with low counts of landing sites (indication of recombination specificity), showing efficient integration into less than 10 genomic loci (FIG. 9A). Using a regression analysis, representative LSRs from cluster 16 and 85 were identified as outliers that demonstrate efficient and specific integration in the human genome. Cluster 16 has 3 integration sites with over 50% at its top integration locus, and cluster 85 has 2 sites with over 99% at its top integration locus (FIG. 9B).

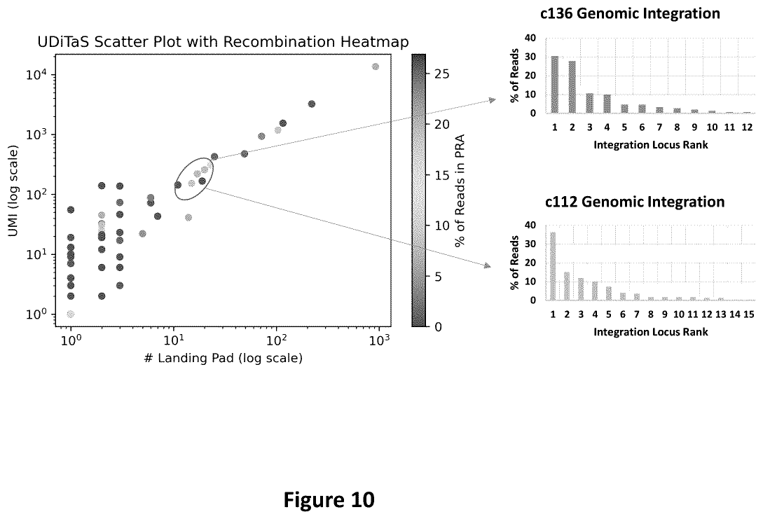

To examine LSR clusters in both the context of plasmid recombination and genomic integration, the plasmid recombination data was overlayed via heat map onto the genomic integration data (FIG. 10). Clusters 136 and 112 are highly efficient across both functional assays, respectively demonstrating twelve and fifteen integration loci with over 80% of integrations occurring across the top 5 integration sites (FIG. 10).

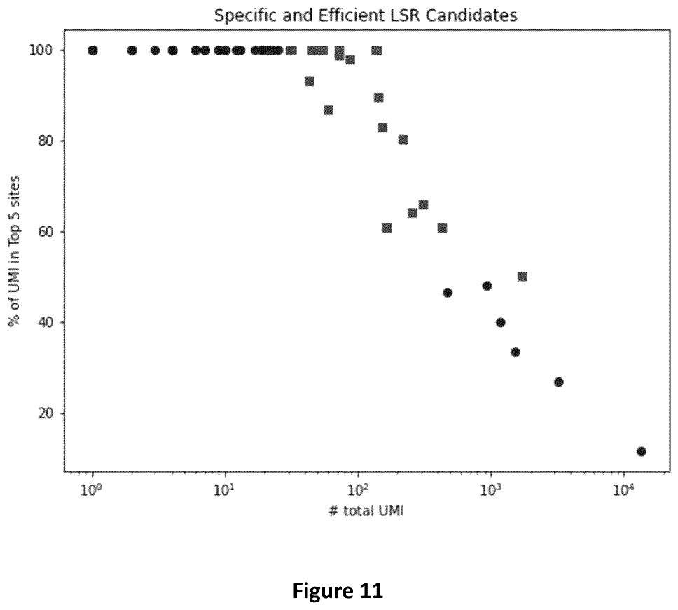

Further results from the pooled genomic integration assay are shown in FIG. 11 and Table 5, which show (for each cluster) the percent of UMI in the top 5 genomic integration sites (y-axis) and the total number of UMI (x-axis). This highlights clusters with specific targeting at fewer genomic sites. Select LSRs shown in red squares in FIG. 11 have a % of UMI in Top 5 sites>50 and a #total UMI>30. The integration sites of these clusters were interrogated and functionally annotated (Table 4). Of note, the integration sites for the clusters identified in previous analyses (clusters 16, 85, 112, and 136) are also described.

TABLE 5
UMI Top 5 Landing Sites

| lsr_cluster | total_umi_count | top5_umi_fraction |
|---|---|---|
| Dn29 | 60 | 86.67 |
| PRO418 | 1 | 100 |
| PRO426 | 1698 | 50.16 |
| PRO439 | 2 | 100 |
| Pa01 | 1 | 100 |
| c3 | 7 | 100 |
| c6 | 45 | 100 |
| c10 | 9 | 100 |
| c11 | 72 | 98.62 |
| c12 | 25 | 100 |
| c16 | 137 | 100 |
| c18 | 32 | 100 |
| c19 | 256 | 64.07 |
| c25 | 3215 | 26.87 |
| c27 | 31 | 100 |
| c29 | 7 | 100 |
| c33 | 1 | 100 |
| c36 | 13661 | 11.58 |
| c39 | 6 | 100 |
| c41 | 1 | 100 |
| c42 | 1535 | 33.29 |
| c45 | 4 | 100 |
| c46 | 23 | 100 |
| c49 | 1 | 100 |
| c51 | 1175 | 39.91 |
| c52 | 4 | 100 |
| c59 | 2 | 100 |
| c60 | 19 | 100 |
| c72 | 473 | 46.51 |
| c75 | 46 | 99.99 |
| c76 | 55 | 100 |
| c77 | 425 | 60.95 |
| c83 | 10 | 100 |
| c84 | 166 | 60.84 |
| c85 | 139 | 100 |
| c89 | 2 | 100 |
| c93 | 309 | 66.02 |
| c94 | 21 | 100 |
| c96 | 19 | 100 |
| c98 | 17 | 99.99 |
| c99 | 931 | 47.91 |
| c100 | 19 | 100 |
| c103 | 88 | 97.72 |
| c104 | 43 | 93.03 |
| c109 | 1 | 100 |
| c111 | 73 | 100 |
| c112 | 219 | 80.37 |
| c113 | 13 | 100 |
| c117 | 3 | 100 |
| c134 | 12 | 100 |
| c136 | 152 | 82.9 |
| c140 | 143 | 89.51 |
| c145 | 6 | 100 |
| c150 | 9 | 100 |
| c152 | 1 | 100 |
| c154 | 22 | 100.01 |
| c157 | 13 | 100 |
| c158 | 10 | 100 |
| c159 | 4 | 100 |

### REFERENCES

Alberts, B., Johnson, A., Lewis, J., et al. (2002). Site-Specific Recombination. Molecular Biology of the Cell. 4th edition.
Altschul SF, G. W. (1990). Basic local alignment search tool. Journal of Molecular Biology 215(3), 403.
Bai, H., Sun, M., Hatfull, G., Grindley, N., & Marko, J. (2011). Single-molecule analysis reveals the molecular bearing mechanism of DNA strand exchange by a serine recombinase. PNAS 108(18), 7419.
Edgar, R. C. (2010). Search and clustering orders of magnitude faster than BLAST. Bioinformatics 26(19), 2460.
Fennell, T., Zhang, D., Isik, M., Wang, T., Gotta, G., Wilson, C. J., & Marco, E. (2021). CALITAS: A CRISPR-Cas-aware Aligner for In silico off-Target Search. The CRISPR Journal 4(2), 264.
Giannoukos, G., Ciulla, D. M., Marco, E. et al. (2018). UDiTaS™, a genome editing detection method for indels and genome rearrangements. BMC Genomics 19, 212.
Grindley, N., Whiteson, K., & Rice, P. (2006). Mechanisms of Site-Specific Recombination. Annual Review of Biochemistry 75, 567.
Hyatt, D., Chen, G.-L., Locascio, P., Land, M., Larimer, F., & Hauser, L. (2010). Prodigal: prokaryotic gene recognition and translation initiation site identification. BMC Bioinformatics 11, 1.
Keenholtz, R., Rowland, S., Boocock, M., Stark, W. M., & Rice, P. (2011). Structural Basis for Catalytic Activation of a Serine Recombinase. Structure 19(6), 799.
Kim, A., Ghosh, P., Aaron, M., Bibb, L. A., Jain, S., & Hatfull, G. (2003). Mycobacteriophage Bxb1 integrates into the Mycobacterium smegmatis groELI gene. Molecular Microbiology 50(2), 463.
Lambert, J. M., Bongers, R. S., & Kleerebezem, M. (2007). Cre-lox-Based System for Multiple Gene Deletions and Selectable-Marker Removal in Lactobacillus plantarum. Applied and Environmental Microbiology 73(4), 1126.
Lees, J. A., Harris, S. R., Tonkin-Hill, G., Gladstone, R. A., Lo, S. W., Weiser, J. N., Corander, J., Bentley, S. D., & Croucher, N. J. (2019). Fast and flexible bacterial genomic epidemiology with PopPUNK. Genome Research 29(2), 304.
Li H. & Durbin, R. (2009). Fast and accurate short read alignment with Burrows-Wheeler transform. Bioinformatics 25(14), 1754.
Merrick, C. A., Zhao, J., & Rosser, S. J. (2018). Serine Integrases: Advancing Synthetic Biology. ACS Synthetic Biology 7(2), 299.
Olorunniji, F. J., Rosser, S. J., & Stark, W. M. (2016). Site-specific recombinases: molecular machines for the Genetic Revolution. Biochemical Journal 473(6), 673.
Smith, M. C., & Thorpe, H. M. (2002). Diversity in the serine recombinases. Molecular Microbiology 44(2), 299.
Swalla, B. M., Gumport, R. I., & Gardner, J. F. (2003). Conservation of structure and function among tyrosine recombinases: homology-based modeling of the lambda integrase core-binding domain. Nucleic Acids Research 31(3), 805.
Van Duyne, G. D., & Rutherford, K. (2013). Large serine recombinase domain structure and attachment site binding. Critical Reviews in Biochemistry and Molecular Biology 48(5), 476.
Zhang, Z., & Lutz, B. (2002). Cre recombinase-mediated inversion using lox66 and lox71: method to introduce conditional point mutations into the CREB-binding protein. Nucleic Acids Research 30(17), e90.

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes as described herein. The publications, websites and other reference materials referenced herein to describe the background of the invention and to provide additional detail regarding its practice are hereby incorporated by reference in their entireties.

1. A method for integrating an exogenous nucleic acid (e.g., exogenous DNA) into a human genome, the method comprising:
contacting a human cell with:
an exogenous nucleic acid (e.g., exogenous DNA) comprising a nucleic acid sequence of interest and a first attachment site and
a serine recombinase or a polynucleotide encoding the serine recombinase,
wherein the human genome comprises a second attachment site and recombination between the first and second attachment sites results in integration of the exogenous nucleic acid (e.g., exogenous DNA) into the human genome.

2. The method of claim 1, wherein the exogenous nucleic acid (e.g., exogenous DNA) is up to 5 kb, up to 25 kb, up to 50 kb, up to 75 kb, up to 100 kb, up to 150 kb, up to 200 kb, up to 250 kb, or up to 300 kb in size.

3. The method of claim 1, wherein the first attachment site is or comprises a donor attachment (attD) site, and wherein the attD site comprises an attB sequence or an attP sequence.

4. (canceled)

5. The method of claim 3, wherein the first attachment site comprises a nucleic acid sequence at least 50% identical to an attB or attP sequence selected from Table 1.

6. The method of claim 1, wherein the second attachment site is or comprises an acceptor attachment (attA) site, and wherein the attA site comprises an attB sequence, an attP sequence, or an attH sequence.

7. (canceled)

8. The method of claim 7, wherein the second attachment site comprises a nucleic acid sequence at least 50% identical to: an attB sequence selected from Table 1, an attP sequence selected from Table 1, or an attH sequence selected from Table 1.

9. The method of claim 1, wherein the serine recombinase comprises an amino acid sequence at least 80% identical to a sequence selected from Table 1.

10. The method of claim 1, wherein the serine recombinase comprises:
an amino-terminal catalytic domain,
a recombinase domain, and
a DNA-binding zinc ribbon domain,
wherein, according to UCLUST algorithm analysis, the amino-terminal catalytic domain, the recombinase domain, and the DNA-binding zinc ribbon domain comprise amino acid sequences at least 90% identical to a sequence selected from Table 1, wherein the sequence selected from Table 1 comprises an amino-terminal catalytic domain, a recombinase domain, and a DNA-binding zinc ribbon domain.

11. The method of claim 1, wherein the serine recombinase comprises:
an amino-terminal catalytic domain,
a recombinase domain, and
a DNA-binding zinc ribbon domain,
wherein, according to UCLUST algorithm analysis, the amino-terminal catalytic domain, the recombinase domain, and the DNA-binding zinc ribbon domain comprise amino acid sequences at least 90% identical to a sequence selected from Table 2, wherein the sequence selected from Table 2 comprises an amino-terminal catalytic domain, a recombinase domain, and a DNA-binding zinc ribbon domain.

12. The method of claim 1, wherein the serine recombinase is a recombinase selected from cluster 2, 3, 6, 7, 11, 12, 14, 16, 75, 76, 82, 85, 93, 103, 104, 111, 112, 136, 140, 144, 148, or 152 as identified in Table 1.

13. The method of claim 1, wherein the serine recombinase comprises an amino acid sequence at least 80% identical to a sequence selected from SEQ ID NO: 58926, SEQ ID NO: 10611, SEQ ID NO: 33021, SEQ ID NO: 40191, SEQ ID NO: 5681, SEQ ID NO: 36231, SEQ ID NO: 34841, SEQ ID NO: 9906, SEQ ID NO: 21701, SEQ ID NO: 7466, SEQ ID NO: 57456, SEQ ID NO: 41066, SEQ ID NO: 41186, SEQ ID NO: 21126, SEQ ID NO: 1191, SEQ ID NO: 35081, SEQ ID NO: 18926, SEQ ID NO: 51806, SEQ ID NO: 58376, SEQ ID NO: 29771, SEQ ID NO: 21276, or SEQ ID NO: 36986.

14. The method of claim 1, wherein the serine recombinase, the first attachment site, and the second attachment site comprise sequences at least 80% identical to sequences that have the same system ID in Table 1.

15. The method of claim 1, wherein the polynucleotide encoding the serine recombinase is or comprises mRNA.

16. The method of claim 1, wherein the polynucleotide encoding the serine recombinase is or comprises DNA.

17. The method of claim 1, wherein the polynucleotide encoding the serine recombinase is operably linked to a promoter that is active in the human cell.

18. The method of claim 1, wherein the exogenous nucleic acid (e.g., exogenous DNA) is or comprises a plasmid, a nanoplasmid, a mini-circle, or doggybone DNA (dbDNA).

19. The method of claim 18, wherein the exogenous nucleic acid (e.g., exogenous DNA) is delivered to the human cell in a lipid nanoparticle (LNP), an adeno-associated virus (AAV), a lentivirus, a virus-like particle (VLP), an exosome, a cationic nanoparticle, or a dendrimer.

20. The method of claim 1, wherein the exogenous nucleic acid (e.g., exogenous DNA) and the polynucleotide encoding the serine recombinase are delivered to the human cell in an LNP, and wherein the polynucleotide encoding the serine recombinase is or comprises mRNA.

21. The method of claim 1, wherein the human cell is or comprises: an osteoblast, a chondrocyte, an adipocyte, a skeletal muscle cell, a cardiac muscle cell, a neuron, an astrocyte, an oligodendrocyte, a Schwann cell, a retinal cell, a corneal cell, a skin cell, a monocyte, a macrophage, a neutrophil, a basophil, an eosinophil, an erythrocyte, a megakaryocyte, a dendritic cell, a T-lymphocyte, a B-lymphocyte, an NK-cell, a gastric cell, an intestinal cell, a smooth muscle cell, a vascular cell, a bladder cell, a pancreatic alpha cell, a pancreatic beta cell, a pancreatic delta cell, a liver cell (e.g., a hepatocyte, a hepatic stellate cell, a Kupffer cell, or a liver sinusoidal endothelial cell), a renal cell, an adrenal cell, a lung cell, a mesenchymal stem cell, a hematopoietic stem cell, a hematopoietic progenitor cell, a neuronal stem cell, a retinal stem cell, a cardiac muscle stem cell, a skeletal muscle stem cell, an adipose tissue derived stem cell, a chondrogenic stem cell, a liver stem cell, a kidney stem cell, a pancreatic stem cell, an embryonic stem cell, an induced pluripotent stem cell, or a fate-converted stem or progenitor cell.

22. A transgenic human cell obtained by the method of claim 1.

23. A transgenic human cell obtained by culturing the transgenic human cell of claim 22.

24. A method for obtaining integration of an exogenous nucleic acid (e.g., exogenous DNA) comprising a nucleic acid sequence of interest and a first attachment site into a human genome comprising a second attachment site, the method comprising:
contacting the first attachment site with the second attachment site in the presence of a serine recombinase,
wherein the contacting step results in recombination between the first and second attachment sites, and
wherein recombination between the first and second attachment sites results in integration of the exogenous nucleic acid (e.g., exogenous DNA) into the human genome.

25-36. (canceled)

37. A system for integrating an exogenous nucleic acid (e.g., exogenous DNA) comprising a nucleic acid sequence of interest into a human genome, the system comprising:
an exogenous nucleic acid (e.g., exogenous DNA) comprising a nucleic acid sequence of interest and a first attachment site, and
a serine recombinase or a polynucleotide encoding the serine recombinase.

38-55. (canceled)
